(19) United States
(12) Patent Application Publication
(10) Pub. No.: US 2009/0298103 A1
(43) Pub. Date: Dec. 3, 2009

(54) Predicting hemostatic risk; dependence on plasma composition

(75) Inventors: Kenneth G. Mann, Grand Isle, VT (US); Kathleen B. Ziedins, Waterbury Center, VT (US); Thomas Orfeo, Westford, VT (US); Matthew F. Hockin, Salt Lake City, UT (US); Stephen J. Everse, South Burlington, VT (US)

Correspondence Address: EDWARDS ANGELL PALMER & DODGE LLP, P.O. BOX 55874, BOSTON, MA, 02205, US

(73) Assignee: The University of Vermont and State Agriculture College, Burlington, VT (US)

(21) Appl. No.: 12/454,677

(22) Filed: May 20, 2009

Related U.S. Application Data
(60) Provisional application No. 61/054,503, filed on May 20, 2008.

(51) Int. Cl. C12Q 1/56; G01N 33/48

(52) U.S. Cl. 435/13; 702/19

(57) ABSTRACT

Featured are methods for assessing hemostatic risk including the risk for ACS. Such methods include acquiring blood/plasma composition based on a biological sample obtained from a subject, determining parameters associated with blood clotting, simulating in silico blood clotting using the determined parameters and comparing the results of such simulation to a reference and to determine the hemostatic risk from said comparing. In further embodiments, such methods further include selecting a treatment regime or protocol based on the results of such comparing. In yet further embodiments, such methods further include assessing the efficacy of medicants, drugs and the like of a given treatment protocol such as by simulating in silico the application of such medicants, drugs and the like.

This application claims the benefit of U.S. Provisional Application Ser. No. 61/054,503 filed May 20, 2008 and is related to co-pending U.S. application Ser. No. 10/507,661, which was a National Stage Filing of PCT Application No. PCT/US03/07379, filed Mar. 10, 2003, the teachings of all being incorporated herein by reference in their entirety.

### STATEMENT REGARDING FEDERALLY-SPONSORED RESEARCH

The present invention was supported by grants from the USDOE grant number FG0200ER45828 and National Institute of Health (NIH), grant number HL46703. The U.S. Government may have certain rights to the present invention.

### FIELD OF INVENTION

The present invention relates to methods for assessing and determining hemostatic risk and more particularly to methods for using numerical techniques for simulating in silico blood clotting reactions as a mechanism for such assessing and determining.

### BACKGROUND OF THE INVENTION

Millions of patients each year in the United States are admitted to an emergency department with chest pain, often resulting from the progression of CAD from an asymptomatic fibroatheromatous plaque into a high-risk unstable lesion. The generation of the enzyme thrombin from its precursor prothrombin is the central event of the blood coagulation process, essential for hemostasis and the culprit in thrombosis [Mann, K. G., Butenas, S., and Brummel, K. E. (2003) Arterioscler. Thromb. Vasc. Biol. 23, 17-25; Mann, K. G., Brummel, K., and Butenas, S. (2003) Journal of Thrombosis and Haemostasis 1, 1504-1514]. Congenital diseases associated with the absence or reduced production of thrombin (hemophilias) represent important clinical problems that, fortunately, are rare [Gitschier, J. (1991) Ann. N.Y. Acad. Sci. 614, 89-96; McGraw, R. A., Davis, L. M., Lundblad, R. L., Stafford, D. W., and Roberts, H. R. (1985) Clin. Haematol. 14, 359-383; Cawthern, K. M., van't Veer, C., Lock, J. B., DiLorenzo, M. E., Branda, R. F., and Mann, K. G. (1998) Blood 91, 4581-4592; Butenas, S., Brummel, K. E., Branda, R. F., Paradis, S. G., and Mann, K. G. (2002) Blood 99, 923-930].

The unregulated production of thrombin in an inappropriate location that leads to a thrombotic occlusion, however, is a frequently encountered problem. Thrombosis in the venous circulation and the embolization of venous clots were recognized to be important contributors to pathology for over a century [Rosendaal, F. R. (1999) Lancet 353, 1167-1173]. More recently, the significance of clot formation in the arterial circulation led to the development of both molecular and mechanical interventions to disrupt formed clots, which cause myocardial infarction and stroke [Collen, D. and Haber, E. (1999) The fibrinolytic system and thrombolytic therapy. In Chien, K. R., editor. W.B. Saunders, Philadelphia; Lettino, M., Falcone, C., and Tavazzi, L. (2005) Ital. Heart J. 6, 1-8].

Thrombin's contribution to venous thrombosis is clearly paramount as evidenced by abundant clinical data demonstrating the efficacy of direct or indirect thrombin inhibitors in primary and, secondary venous thromboembolic prophylaxis [Kwaan, H. C. and Samama, M. M. (2004) Expert. Rev. Cardiovasc. Ther. 2, 511-522; Heit, J. A. (2003) Chest 124, 40S-48S]. In addition, while platelet inhibition plays a major role in arterial thrombosis [Messmore, H. L., Jr., Jeske, W. P., Wehrmacher, W., Coyne, E., Mobarhan, S., Cho, L., Leya, F. S., and Moran, J. F. (2005) Hematol. Oncol. Clin. North Am. 19, 87-117, vi], thrombocytopenia and platelet inhibitors appear to play limited roles in attenuating venous thromboembolism (VTE) [Geerts, W. H., Pineo, G. F., Heit, J. A., Bergqvist, D., Lassen, M. R., Colwell, C. W., and Ray, J. G. (2004) Chest 126, 338S-400S]. VTE in both males and females shows a sharp age dependency [Heit, J. A. (2003) Chest 124, 40S-48S; Silverstein, M. D., Heit, J. A., Mohr, D. N., Petterson, T. M., O'Fallon, W. M., and Melton, L. J., III (1998) Arch. Intern. Med. 158, 585-593], and is a source of growing concern for both health and economic reasons in our aging population. In addition, while a variety of congenital polymorphisms and various pro- and anti-coagulant factor levels are associated with VTE, presently no blood indicators signal pharmacologic intervention prior to an acute event [Mann, K. G., Brummel-Ziedins, K., Undas, A., and Butenas, S. (2004) J. Thromb. Haemost. 2, 1727-1734].

Identification of blood borne markers or methods which would be informative concerning the level of progression of CAD or the likelihood of an acute event has remained elusive. Risk factors that have been established for venous thrombosis have not been transferable easily to the prediction of risk of arterial thrombosis. By focusing on thrombophilic deficiency states highly correlated with VTE (factor V^{Leiden} [Doggen, C. J., Cats, V. M., Bertina, R. M., and Rosendaal, F. R. (1998) Circulation 97, 1037-1041; Nicolaes, G. A. and Dahlback, B. (2003) Hematol. Oncol. Clin. North Am. 17, 37-61, vi], protein C [Lu, D., Bovill, E. G., and Long, G. L. (1994) J. Biol. Chem. 269, 29032-29038; Reiner, A. P., Siscovick, D. S., and Rosendaal, F. R. (2001) Thromb. Haemost. 85, 584-595], protein S [Patel, R. K., Ford, E., Thumpston, J., and Arya, R. (2003) Thromb. Haemost. 90, 835-838; Bhattacharyya, M., Kannan, M., Chaudhry, V. P., and Saxena, R. (2003) Indian J. Pathol. Microbiol. 46, 621-624] and AT [van Boven, H. H. and Lane, D. A. (1997) Semin. Hematol. 34, 188-204; Harper, P. L., Luddington, R. J., Daly, M., Bruce, D., Williamson, D., Edgar, P. F., Perry, D. J., and Carrell, R. W. (1991) Br. J. Haematol. 77, 360-364], Vossen et al. showed that hereditary thrombophilia can contribute to the risk of arterial disease; thus relating specific defects in natural anticoagulant systems that regulate thrombin generation in venous thrombosis to arterial disease [Vossen, C. Y. and Rosendaal, F. R. (2006) J. Thromb. Haemost. 4, 916-918].

A meta-analysis by Ye et al. on seven hemostatic genetic variants showed that factor V^{Leiden} and G20210A variant of the prothrombin gene are potentially associated with the risk of CAD [Ye, Z., Liu, E. H., Higgins, J. P., Keavney, B. D., Lowe, G. D., Collins, R., and Danesh, J. (2006) Lancet 367, 651-658]. Analyses aimed at assessing the impact of blood composition on arterial thrombosis, in individuals without identified abnormalities in coagulation factor levels, have most notably identified factor VIII [Bank, I., Libourel, E. J., Middeldorp, S., Hamulyak, K., van Pampus, E. C., Koopman, M. M., Prins, M. H., van der, M. J., and Buller, H. R. (2005) J. Thromb. Haemost. 3, 79-84; Malecki, R. and Adamiec, R. (2006) Postepy Hig. Med. Dosw. (Online) 60, 602-608; Martinelli, I. (2005) Semin. Hematol. 42, 49-55; Cortellaro, M., Boschetti, C., Cofrancesco, E., Zanussi, C., Catalano, M., de Gaetano, G., Gabrielli, L., Lombardi, B., Specchia, G., Tavazzi, L., and (1992) Arterioscler. Thromb. 12, 1063-1070; Folsom, A. R., Wu, K. K., Rosamond, W. D., Sharrett, A. R., and Chambless, L. E. (1997) Circulation 96, 1102-1108; Tracy, R. P., Bovill, E. G., Yanez, D., Psaty, B. M., Fried, L. P., Heiss, G., Lee, M., Polak, J. F., and Savage, P. J. (1995) Arterioscler. Thromb. Vasc. Biol. 15, 1269-1279] and fibrinogen [Morange, P. E., Bickel, C., Nicaud, V., Schnabel, R., Rupprecht, H. J., Peetz, D., Lackner, K. J., Cambien, F., Blankenberg, S., and Tiret, L. (2006) Arterioscler. Thromb. Vasc. Biol. 26, 2793-2799].

Although studies have identified blood borne products derived from coagulant activity (i.e. soluble fibrin as potentially useful markers of ongoing or recent arterial thrombosis, the heterologous presence of any of these circumstances in asymptomatic individuals does not correlate with the need for clinical intervention [Morange, P. E., Bickel, C., Nicaud, V., Schnabel, R., Rupprecht, H. J., Peetz, D., Lackner, K. J., Cambien, F., Blankenberg, S., and Tiret, L. (2006) Arterioscler. Thromb. Vasc. Biol. 26, 2793-2799; Derhaschnig, U., Laggner, A. N., Roggla, M., Hirschl, M. M., Kapiotis, S., Marsik, C., and Jilma, B. (2002) Clin. Chem. 48, 1924-1930; Carville, D. G., Dimitrijevic, N., Walsh, M., Digirolamo, T., Brill, E. M., Drew, N., and Gargan, P. E. (1996) Clin. Chem. 42, 1537-1541; Laurino, J. P., Pelletier, T. E., Eadry, R., and Kounavis, A. (1997) Ann. Clin. Lab Sci. 27, 338-345], TAT, prothrombin F1+2) as well as the status of platelets [Linden, M. D., Furman, M. I., Frelinger, A. L., III, Fox, M. L., Barnard, M. R., Li, Y., Przyklenk, K., and Michelson, A. D. (2007) J. Thromb. Haemost. 5, 761-765].

Most investigators suggest pathologies associated with thrombotic disease are multifactorial in nature. Thus, while many independent risk factors have been identified with the blood composition of individuals with thrombosis, no procedure exists which will compile composition data into algorithms instructive with respect to risk management.

The development of a physical understanding of any natural process can be divided into three interactive domains as illustrated in FIG. 10. Many laboratories have contributed to the content and interrelationship between these domains. The information in each domain influences the contents of the other domains, leading to the identification of new components and processes which in turn alter the descriptions of the physiology and pathophysiology of blood coagulation.

The complex catalysts, which participate in the generation of thrombin via the Tf pathway are composed of a serine protease interacting with a receptor/cofactor protein, which are anchored to a discreet surface. In most cases the surface is the membrane of an activated cell. The key-initiating event in the generation of thrombin depends upon the interaction of normally cryptic, membrane bound, Tf with plasma fVIIa. The latter is preexistent in blood at approximately 1-2% of the total fVII concentration (10 nM). While the source and presentation of active Tf is controversial, the damage or cytokine-related presentation of the active Tf trigger for the process is essential. Plasma fVIIa appears to possess the appropriate catalytic machinery to display the active site of an effective serine protease, but does not express proteolytic activity unless it is bound to Tf on a membrane. Thus, naked fVIIa at natural biological concentrations has no significant activity toward either fIX or fX prior to binding to Tf. The defective active site also allows fVIIa to escape inhibition by the high concentrations of antithrombin-III (AT-III) and other inhibitors present in blood. The fVIIa-Tf protein-protein interaction switches on the active site of fVIIa by increasing the k_{cat} of the enzyme and increases the rate of fX activation by four orders of magnitude. This latter increase is the result of the aforementioned improvement in catalytic efficiency and the membrane binding of fIX and fX.

Knowledge of the Tf molecule and its function(s) have been primarily obtained in studies of recombinant forms of Tf which include full length (1-263), the membrane bound (1-242) and the extracellular domain (1-218).

The fVIIa-Tf complex (extrinsic factor Xase) catalyzes the activation of both fix and fX, the latter initially being the more efficient substrate. Thus, the initial product formed by the “extrinsic factor Xase” is fXa. The fIX zymogen is a competitive substrate with fX and requires two peptide bond cleavages (R145, R180) for activity. While both of these cleavages are catalyzed by fVIIa-Tf, fXa, bound to a membrane, they can provide one of the two required cleavages (R145) to produce the intermediate fIXα. Thus, this feedback cleavage by membrane-bound fXa enhances the rate of generation of fIXα, which is completed with the second bond cleavage (R180) by fVIIa-Tf.

The initial fXa produced when bound to a membrane activates small (pM) amounts of prothrombin to thrombin, albeit rather inefficiently. This initial thrombin is essential to the acceleration of the process by serving as the activator for platelets, fV and fVIII. Once fVIIIa is formed, the fIXa generated by fVIIa-Tf combines with fVIIIa on the activated platelet membrane to form the “intrinsic factor Xase” which becomes the major activator of fX.

The fIXa-fVIIa complex is 10⁵-10⁶-fold more active than fIXa alone as a fX activator and 50-fold more efficient than fVIIa-Tf in catalyzing fX activation. In addition, fVIIa-Tf is under the control of Tf pathway inhibitor (TFPI); thus, the bulk of fXa is ultimately produced by fIXa-fVIIIa.

FXa combines with fVa on activated platelet membrane surfaces at specific receptor sites and this fXa-fVa “prothrombinase” catalyst converts prothrombin to thrombin. “Prothrombinase” is ˜300,000-fold more active than fXa alone in catalyzing prothrombin activation.

The coagulation system is under tight regulation by stoichiometric and dynamic inhibition systems. The Tf concentration threshold for reaction initiation is steep and the ultimate amount of thrombin produced is largely regulated by the concentrations of plasma procoagulants and the stoichiometric inhibitors TFPI and AT-III and the constituents of the dynamic inhibition processes.

The principal influence of TFPI is to block the fVIIa-Tf-fXa product complex, thus effectively neutralizing the “extrinsic factor Xase” and eliminating this catalyst's generation of both fXa and fIXa. TFPI function eliminates the initial production of fXa by the “extrinsic factor Xase.” the low abundance (˜2.5 nM), high affinity TFPI, however, can only delay the hemostatic reaction. The TFPI in blood is increased by heparin, which releases the endothelial cell bound inhibitor. The lower affinity, stoichiometric inhibitor AT-III is normally present in plasma at over twice the concentration (3.4 μM) of any potential target enzyme generated by the Tf pathway. AT-III is an effective neutralizer of all the procoagulant serine proteases. The targets of AT-III are primarily the uncomplexed enzyme products of these reactions. α₂-macroglobulin (α₂M) is also reported to account for some thrombin inhibition. α₁-antitrypsin (α₁AT) probably also contributes.

To initiate the dynamic protein C (PC) system, the product enzyme thrombin binds to constitutively present vascular thrombomodulin (Tm) and activates PC, which is presented by the endothelial cell PC receptor (EPCR), to its activated species APC. APC competitively binds with both fVIIa and fVa interfering with the formation of the “intrinsic factor Xase” and the “prothrombinase”, initially by competition with fXa and fIXa and ultimately by cleaving the cofactors to eliminate these complexes. Thus, the combinations of TFPI, the PC system and AT-III cooperate to produce steep Tf concentration thresholds, acting like digital “switches”, allowing or blocking significant thrombin formation. The delay incurred by TFPI and the slower inhibitions by AT-III and the APC system control the level of Tf required to overcome the reaction threshold. TFPI is releasable while Tm is constitutively present on the vascular endothelium. However, the relative concentrations of these two important regulators throughout the vascular system remain ambiguous.

While the foregoing has focused on the primary elements of the Tf pathway, the absence of whose components are associated with pathology in humans and transgenic knockout mice. However, other components play roles, which are less well established. In humans, the zymogen fXI, present in plasma and possibly also in platelets, is variably associated with hemorrhagic pathology. FXI is a substrate for thrombin and has been invoked in a so-called “revised pathway of coagulation”, serving as another contributor to fX activation. Studies of blood from individuals with fXI deficiency illustrate the importance of the feedback activation of fXI but only at very low (≦1 pM) Tf concentrations. At moderate concentrations of Tf (5-10 pM), fXI has little or no effect on thrombin generation in whole blood in vitro. FXI knockout mice do not display spontaneous hemorrhagic pathology. The variability of pathology in humans is most likely a reflection of the nature and extent of the vascular lesion in fXI deficient individuals.

While fXII, prekallekrein and high molecular weight kininogen do not appear to be fundamental to the process of hemostasis, the contributions of contact pathway elements to coagulation and fibrinolysis remain open, thus requiring further experimentation to resolve these issues, especially in the pathology of thrombosis.

Protein S (PS) is a component not appropriately dealt with in present reconstruction studies. Homozygous PS deficiency is clearly a grave prothrombotic state. Infants identified with homozygous PS deficiency do not survive, succumbing to purpura fulminans shortly after birth. However, during the 25 years following its discovery, only a poor and controversial understanding of PS function has been developed. PS has cofactor activity for APC, enhancing cleavage at R306 in fVa. It has also been proposed to be a direct inhibitor of fXa and “prothrombinase”. It was proposed to be a membrane inhibitor. We have confirmed that conclusion in studies that showed the molecule would inhibit prothrombin activation, with inhibition alleviated by phospholipids or platelets. More recently, a report appeared identifying an inhibitory quality of PS using the endogenous thrombin potential assay. PS polymerizes in the absence of Ca⁺⁺ and depolymerizes in the presence of Ca⁺⁺, thus coagulation assays in citrated plasma initiated by Ca⁺⁺ addition are compromised. In plasma, PS is found both free and in complex with C4BP (the binding protein for complement factor C4b). PS is also susceptible to cleavage by thrombin and fXa. The controversial nature of PS “activities” has made it a difficult research subject although its clinical significance is clear.

Like PS the physiologic role of protein Z (PZ) is undetermined. PZ, an enzymatically inert, vitamin K dependent protein, circulates in plasma in complex with a serpin, the PZ dependent protease inhibitor (ZPI). Biochemical studies indicate that PZ functions as a cofactor for ZPI, potentiating its inhibition of fXa.

The use of anticoagulants is central to the management of chronic cardiovascular disease, as well as in acute settings including therapeutic interventions in response to the occurrence of occlusive arterial or venous thrombi and in a broad range of surgical procedures where the procoagulant response must be transiently suppressed. Historically, two agents have been widely used. Warfarin, in use since 1941, has been employed for long-term anticoagulation of individuals. Unfractionated heparin (UFH), a heterogeneous mixture of sulfated, branched glycosaminoglycans (9-90 residues) has been the anticoagulant of choice since the 1930's for many situations requiring acute interventions. Active heparin molecules, constituting 30-40% of UFH preparations, bind to and conformationally activate the stoichiometric plasma protease inhibitor antithrombin (AT), increasing its reactivity with a number of catalysts critical to the procoagulant response, including thrombin (˜10³ fold), factor(f) Xa (˜10² to 10⁴ fold), and fIXa (˜10⁶ fold). Thus anticoagulation with UFH is achieved by increasing the potency of an abundant endogenous inhibitor with broad target specificity. It represents an amplification of the natural, localized anticoagulant mechanism provided by the conformational activation of AT by endothelial cell surface heparan sulfate proteoglycans in the vicinity of a vascular injury. The achieved level of UFH anticoagulation reflects the percentage of the circulating AT pool that is complexed with heparin. For example, plasma levels of UFH considered to be appropriate in some acute settings, from 0.35 to 0.7 U/mL (12), represent “activation” of only 5 to 10% of the circulating AT pool.

The clinical use of UFH is complicated by a number of well documented factors which collectively can lead to either insufficient or excessive anticoagulation of an individual. These include its complex pharmacokinetics properties, technical problems in monitoring heparin levels, the phenomena of heparin resistance, osteopenia and a significant incidence of heparin induced thrombocytopenia. These long appreciated shortcomings of UFH and the diversity of clinical settings requiring anticoagulation therapy of varying intensities have prompted the ongoing development and clinical evaluation of additional anticoagulant therapeutics. These include fragmented heparin preparations, synthetic heparin analogues, molecules that directly target and inhibit specific procoagulant catalysts such as thrombin, fXa, tissue factor (Tf)-fVIIa, and fIXa and agents that enhance endogenous fibrinolysis. Although a number of these drugs have proven to be equivalent or superior to UFH in specific clinical applications, only low molecular weight heparins have achieved a general applicability.

Anticoagulant therapy is used in two broadly defined settings: prophylactic, where the goal is the suppression of anticipated episodes of Tf-initiated coagulation; and therapeutic, where the goal is the suppression of ongoing (already established) coagulation processes. In practice, for a given anticoagulant, the transition between prophylactic and therapeutic applications is accomplished by adjustment of its plasma concentration. Often, after clinical trials, a given anticoagulant and accompanying regimen is recommended for use in only one of these settings, with additional stratification indicating use with specific patient populations or surgical interventions. Improved in vitro and/or computational methods that could predict relative success in prophylactic or therapeutic settings prior to animal and clinical studies or that anticipated specific usage limitations would be useful.

It thus would be desirable to provide new methods for assessing and determining hemostatic risk. It would be particularly desirable to provide such methods that would utilize numerical techniques for assessing an determining such hemostatic risk. It also would be desirable to provide such methods that can assess the efficacy and speed of given treatment protocols including assessing the efficacy of medicants, drug or gene therapy in such treatment protocols.

### SUMMARY OF THE INVENTION

In the present invention, mathematical modeling and computer simulations are tools for the prediction of the behavior of the blood coagulation system and an individual's response to challenge. The basic idea is that in any individual, procoagulant and anticoagulant factor levels together act to generate a unique coagulation profile (or phenotype) and that mathematical modeling represent a methodology for defining these phenotypes. In this approach the correct ensemble of procoagulant and anticoagulant proteins is identified, the thermodynamic, stoichiometric, and catalytic parameters characterizing their interactions are determined, and the relevant physiologic triggers are identified. These modeling efforts exhibits an ability to adequately describe biochemical events in two closed experimental systems: the well-defined synthetic coagulation proteome and the minimally altered phlebotomy whole blood.

Virchow's triad describes the factors influencing thrombosis: the vessel wall, blood and its flow. Of these, blood has been shown to be a clinical predictor of human health and disease. Though complex in its composition, blood is readily accessible and has been characterized extensively. Therefore, mathematical modeling and computer simulations are useful methodologies to adopt in an effort to characterize the etiology of bleeding and thrombosis. We believe that the levels of an individual's proteins in blood at a given time reflect the sum of developmental, environmental, genetic, nutritional, and pharmacological events. Thus, the resulting ensemble of specific protein levels yields a characteristic phenotype that is representative of the in vivo performance of an individual's hemostatic system when it is challenged.

The present inventions also proceed from the basis that a limited array of coagulation factors, all of which are at levels considered clinically normal, can be integrated by a computational model to predict an individual's potential to generate thrombin and thus be related to that individual's hemostatic risk in a variety of settings. For example, acquired data indicates that hypothetical thrombin generation based upon coagulation factor composition can distinguish between ACS and CAD. Individuals within the ACS population generated significantly higher levels of thrombin at a 50% faster rate resulting in more thrombin over the time course of the TF-initiated coagulation reaction. These prothrombotic thrombin generation profiles of individuals with ACS appear to be driven primarily by collective alterations in factor VIII, antithrombin and prothrombin levels. These results suggest that a limited array of coagulation factor and inhibitor levels, all largely within the accepted normal range for these factors/levels, potentially contribute to the procoagulant phenotype in acute phases of CAD. This supports the concept that “vulnerable” circulating blood with prothrombotic alterations and a vulnerable plaque, a potential source of tissue factor (TF), may both play a role in the development of ACS.

According to more particular aspects of the preset invention, there is featured a method for assessing or determining hemostatic risk of a subject by simulating, in silico, the concentration of thrombin based on biological input of a sample taken from a subject and comparing the results of such a simulation with a reference and determining the presence of hemostatic risk based on such results. In this regard, it should be noted that in contrast to prior art techniques the risk assessment is made in the present invention before the onset of an ACS event. In prior art techniques, after the initial onset, a determination is made as to the risk that another such event could occur because of the presence of factors that indicate that the subject is predisposed for such another occurrence. Thus, treatment in the prior art occurs after the fact, whereas in the present invention prophylactic treatment can be determined based on the simulation. It also is within the scope of the present invention, as described herein, to develop treatment after the fact as well.

According to more particular aspects of the preset invention, there is featured a method for assessing or determining hemostatic risk of a subject. Such a method includes determining the concentrations of a plurality of blood factors in a biologic sample from the subject and simulating in silico the concentration of thrombin from the determined concentrations. Such a method further includes comparing the simulated concentration of thrombin to a reference by a clinician, and determining from the simulated concentration if the subject is predisposed to hemostatic risk. In more particular embodiments, the hemostatic risk is one of ACS or hemophilia.

In more particular embodiments, such determining includes determining the concentrations of three blood factors in a biologic sample from the subject. In yet more particular embodiments, the blood factors are selected from the group consisting of AT, FII, FVIII, Protein C, Protein S, Factor V^{Leiden}, and tissue factor pathway inhibitor (TFPI). In yet more specific embodiments, the three blood factors are one of (a) AT, FII, and FVIII; (b) Factor V^{Leiden}, Protein C and Protein S or (c) TFPI, AT and FVIII.

In yet further aspects of the present invention, such a method further includes, after determining that the subject is predisposed to hemostatic risk, determining a prophylactic treatment to minimize the hemostatic risk to the subject. In yet further embodiments, such a method further includes evaluating the efficacy of the determined prophylactic treatment. More particularly, such methods further includes, after determining the prophylactic treatment, inputting parameters representative of the capacity of the prophylactic treatment to modulate at least one blood factor and repeating said simulating in silico the concentration of thrombin from the determined concentrations and the modulating effect of the determined prophylactic treatment. Such methods also include assessing by the clinician the efficacy of the determined prophylactic treatment to minimize the hemostatic risk and (i) if said assessment provides a satisfactory indication of efficacy, proscribing the prophylactic treatment; and (ii) if said assessment provides an unsatisfactory indication of efficacy; selecting another prophylactic treatment and repeating said steps of inputting. said repeating said simulating and said assessing for the another prophylactic treatment.

Such a prophylactic treatment is at least one of drugs, medicaments, dietary and physical therapy. In more particular embodiments, the drug modulates at least one of the procoagulant factor or the anticoagulant factor of the subject.

In yet further embodiments, such simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation. The computer executable functions include at least one of the following variables: 1) TFPI mediated inactivation of TF-VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIa dissociation/activity loss; 6) binding competition, and kinetic activation steps which exist between tissue factor (TF) and factors VII and VIIa, and 7) activation of factor VII by IIa, factor Xa, and factor IXa.

In yet further aspects, in such methods the hemostatic risk is ACS, and such comparing includes comparing the simulated concentration of thrombin to a reference by the clinician, and determining from the simulated concentration if the subject is predisposed to ACS, wherein a simulated thrombin concentration within one standard deviation of the reference indicates that the subject is predisposed to ACS.

In yet further embodiments, such determining includes determining the concentrations of three blood factors in a biologic sample from the subject, where the blood factors are selected from the group consisting of AT, FII, FVIII, Protein C, Protein S, Factor V^{Leiden}, and tissue factor pathway inhibitor (TFPI). In more particular embodiments, the three blood factors are AT, FII, and FVIII.

In yet further aspects of the present invention, such a method further includes, after determining that the subject is predisposed to hemostatic risk, determining a prophylactic treatment to minimize the hemostatic risk to the subject. In yet further embodiments, such a method further includes evaluating the efficacy of the determined prophylactic treatment. More particularly, such methods further includes, after determining the prophylactic treatment, inputting parameters representative of the capacity of the prophylactic treatment to modulate at least one blood factor and repeating said simulating in silico the concentration of thrombin from the determined concentrations and the modulating effect of the determined prophylactic treatment. Such methods also include assessing by the clinician the efficacy of the determined prophylactic treatment to minimize the hemostatic risk and (i) if such assessing provides a satisfactory indication of efficacy, proscribing the prophylactic treatment; and (ii) if such assessing provides an unsatisfactory indication of efficacy; selecting another prophylactic treatment and repeating said steps of inputting. said repeating said simulating and said assessing for the another prophylactic treatment.

Such a prophylactic treatment is at least one of drugs, medicaments, dietary and physical therapy. In more particular embodiments, the drug modulates at least one of the procoagulant factor or the anticoagulant factor of the subject.

In yet further embodiments, such simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation. The computer executable functions include at least one of the following variables: 1) TFPI mediated inactivation of TF•VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

In more particular aspects of the present invention, such methods include a method for diagnosing ACS in a subject, that includes determining the concentrations of AT, FII, and FVIII in a biologic sample from the subject and simulating, in silico, the concentration of thrombin from the concentrations of AT, FII, and FVIII. Such a method also includes comparing the simulated concentration of thrombin to a reference, wherein a simulated thrombin concentration within one standard deviation of the reference indicates that the subject has ACS. In further aspects/embodiments of the present invention, based on such a comparison, the clinician can determine if the subject is predisposed to the occurrence of a medical condition such as ACS or hemophilia and proscribe a prophylactic treatment protocol to minimize the hemostatic risk to the subject.

In yet further aspects of the present invention, such methods include a method for selecting a treatment in a subject. Such a method includes, simulating in silico the concentration of thrombin from the concentrations of a plurality of blood factors in a biologic sample from the subject; comparing the simulated concentration of thrombin to a reference; and selecting a treatment based on said comparing.

In more particular embodiments, such determining includes determining the concentrations of three blood factors in a biologic sample from the subject. In yet more particular embodiments, the blood factors are selected from the group consisting of AT, FII, FVIII, Protein C, Protein S, Factor V^{Leiden}, and tissue factor pathway inhibitor (TFPI). In yet more specific embodiments, the three blood factors are one of (a) AT, FII, and FVIII; (b) Factor V^{Leiden}, Protein C and Protein S or (c) TFPI, AT and FVIII.

In yet further embodiments, such a method further includes evaluating the efficacy of the determined prophylactic treatment. More particularly, such methods further includes, after determining the treatment, inputting parameters representative of the capacity of the treatment to modulate at least one blood factor and repeating said simulating in silico the concentration of thrombin from the determined concentrations and the modulating effect of the determined treatment. Such a method also include assessing by the clinician the efficacy of the determined treatment and (i) if such assessing provides a satisfactory indication of efficacy, proscribing the treatment; and (ii) if such assessing provides an unsatisfactory indication of efficacy; selecting another treatment and repeating said steps of inputting. said repeating said simulating and said assessing for the another treatment.

Such a treatment is at least one of drugs, medicaments, dietary and physical therapy. In more particular embodiments, the drug modulates at least one of the procoagulant factor or the anticoagulant factor of the subject.

In yet further embodiments, such simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation. The computer executable functions include at least one of the following variables: 1) TFPI mediated inactivation of TF•VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

According to a more particular aspect of the present invention, such methods further include a method for selecting a treatment in a subject, where such a method includes simulating the concentration of thrombin from the concentrations of AT, FII, and FVIII in a biologic sample from the subject and comparing the simulated concentration of thrombin to a reference. Such a method further includes selecting a treatment based on such comparing.

In yet further embodiments, such simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation.

In yet further aspects and embodiments, such method further includes assessing the efficacy of drugs, medicaments and other therapy known to those skilled in the art to provide the expected prophylactic treatment and thus minimize the hemostatic risk. In more particular embodiments such assessing efficacy includes a methodology for screening, in silco, candidate compounds for ability to prevent or treat the underlying medical condition blood clotting. Such a methodology includes pre-selecting a drug, medicament or the like for capacity to modulate at least one blood factor, performing a series of computer executable functions that manipulate input data featuring at least one of blood coagulation formation, expression and propagation variables, the functions generating, as output, a thrombin concentration, wherein the amount of thrombin produced by the drug is taken to be indicative of a compound that prevents or treats the medical condition.

In the foregoing, the computer executable functions include at least one of the following variables: 1) TFPI mediated inactivation of TF•VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

Other aspects and embodiments of the invention are discussed below.

### DEFINITIONS

The instant invention is most clearly understood with reference to the following definitions:

ACS shall be understood to mean or refer to acute coronary syndrome.

APC shall be understood to mean or relate to the activated species of protein C.

CAD shall be understood to mean or refer to stable coronary artery disease.

Computer readable medium shall be understood to mean any article of manufacture that contains data that can be read by a computer or a carrier wave signal carrying data that can be read by a computer. Such computer readable media includes but is not limited to magnetic media, such as a floppy disk, a flexible disk, a hard disk, reel-to-reel tape, cartridge tape, cassette tape or cards; flash memory devices including NVRAN; optical media such as CD-ROM, DVD and writeable compact disc; magneto-optical media in disc, tape or card form; paper media, such as punched cards and paper tape; or on carrier wave signal received through a network, wireless network or modem, including radio-frequency signals and infrared signals.

EPCR shall be understood to mean or relate to endothelial cell PC receptor.

In silico as used herein shall be understood to general means performed on a computer, microprocessor, digital signal processor, processing circuitry, an application specific integrated circuits (ASIC), or the like; via a computer, microprocessor, digital signal processor, processing circuitry, ASIC, or the like; via a computer simulation (e.g., a program executed on a computer, microprocessor, digital signal processor, processing circuitry, ASIC, or the like. In usages more specific to medicine, medical devices, biology and the like, in silico also refers to methods to test biological models, drugs and medical interventions using sophisticated computer models embodied in programs that are executed on a computer, microprocessor, digital signal processor, processing circuitry, ASIC, or the like) rather than expensive laboratory (in vitro) and animal experiments (in vivo)

Pathway shall be understood to generally mean or relate to a plurality of reactions (e.g., chemical reactions, binding reactions, and the like), such as those which are involved in biochemical, cellular, physiological, and/or pathophysiological processes. A pathway may interconnect with, and/or be regulated by one or more other pathways. Preferably, the one or more other pathways also can be modeled as a series of chemical reactions and/or binding reactions.

PC shall be understood to mean or relate to protein C.

PS shall be understood to mean or relate to protein S.

UFH shall be understood to mean or relate to unfractionated heparin.

VTE shall be understood to mean or relate to venous thromboembolism.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a high level flow diagram of an exemplary method according to the present invention for assessing hemostatic risk and also for minimizing risk by determining a treatment protocol.

FIG. 2 is another high level flow diagram of another exemplary method according to the present invention for optimizing the treatment protocol.

FIG. 3 is a block diagram representing a logic sequence of a methodology for simulating in silico to assess hemostatic risk, such as described in PCT Application No. PCT/US03/07379.

FIGS. 4A-D are block diagrams representing the logic sequence of a methodology for the solver feature of FIG. 2, such as also described in PCT Application No. PCT/US03/07379.

FIG. 5 is a listing of equations that are useable in the simulating methodology of FIG. 3. The notation −b 2> signifies a forward reaction dictated by rate constant “2” or k₂ and the notation <1—signifies a reverse reaction dictated by rate constant “1” or k₁. The notation <1-2> indicates and equilibrium expression with a forward rate constant k₂ and a reverse rate constant of k₁. Binding between components is indicated by the =notation, i.e., A+B<1-2>A=B.

FIG. 6 is a tabulation of plasma composition factor levels for ACS and CAD populations and for healthy individuals.

FIGS. 7A-D are graphical views of thrombin simulations and empirical measurements of ACS and CAD populations. Plasma compositions from 28 ACS individuals (FIG. 7A) and 25 CAD individuals (FIG. 7B) were used to generate thrombin simulations over 1200 s. Computationally derived thrombin generation is shown as the mean for the ACS and CAD population (FIG. 7C). The positive standard deviation is shown in gray. Empirical synthetic coagulation proteome was reconstructed on a lipid surface (PC/PS) using the purified factors (F) II, V, VII, VIII, IX, X, antithrombin (AT) and tissue factor pathway inhibitor (TFPI) (FIG. 7D). The concentration of the factor levels was determined from the mean of these factors in the ACS and CAD populations. Mean physiologic concentrations were used as the control.

FIG. 8 is a tabulation of mean simulated thrombin parameters.

FIGS. 9A-D are various graphical views of the normalization of the acute coronary syndrome (ACS) population. Within the ACS population, factor (F) VIII, prothrombin (FII) and antithrombin (AT) were set at mean physiologic concentration (FVIII: 0.7 nM, FII: 1.4 μM, AT: 3.4 μM). The normalized ACS thrombin generation is compared with the control (all factor levels at mean physiologic concentration), mean coronary artery disease and ACS populations.

FIG. 10 is an illustrative view of physical domain relationships.

FIGS. 11A,B are graphical views of the dynamics of thrombin formation in contact pathway inhibited whole blood; FIG. 11A normal versus hemophilia and FIG. 11B thrombin thresholds for procoagulant events.

FIG. 12 is a graphical view of a numerical estimation of relative factor Xa production by the extrinsic factor Xase (♦) and the intrinsic factor Xase (▪).

FIGS. 13 A,B are graphical views of resupply (open symbols) of Tf initiated blood coagulation reaction after the cessation of prothrombin consumption: normal (diamonds) versus hemophilia (squares); where FIG. 3A is the numerical simulation and FIG. 13B is contact pathway suppressed whole blood.

FIG. 14 is an illustrative view using four cartoon panels showing a two compartment model for Tf induced blood coagulation with flow.

FIG. 15 is a graphical view showing simulated time courses of the formation of factor Xa, factor Va, thrombin (IIa), and meizothrombin (m-IIa) in response to a 5 pM tissue factor stimulus.

FIG. 16 is a graphical view of thrombin generation from contact pathway inhibited whole blood at a 20 min time point (mean±SD) in 13 individuals over a 6 month time frame.

FIGS. 17A-D are graphical views of Oral contraceptive (OC) effect on theoretical and empirical thrombin generation. Thrombin generation simulations are shown as the mean and the 95% confidence interval in FIG. 17A for control women with (n=47) and without (n=90) OC use and in FIG. 17B for women with deep venous thrombosis (DVT) with (n=30) and without (n=40) OC use. The mean factor levels of the selected populations shown in FIGS. 17 A,B were recapitulated in a synthetic plasma model FIGS. 17C,D.

FIG. 18 is a graphical view of numerical simulations of thrombin generation in functionally severe hemophilia patients.

FIGS. 19 A,B are graphical views of IVF hormone therapy influence on thrombin generation for two individuals.

FIGS. 20A,B are graphical views of numerical simulations of the effect of factor V Leiden on thrombin generation; FIG. 20A Tf dependence of total thrombin generation: normal (♦) and factor V Leiden (▪) and FIG. 20B Thrombin generation with 25 pM Tf: normal (▪), hemophilia A (≡), hemophilia A plus factor V Leiden (◯).

FIG. 21 is a graphical view of a contact pathway inhibited whole blood clot time as a function of exogenous added Tf in 2 individuals (open and filled bars).

FIG. 22 is an SDS-PAGE under reducing conditions of various Tf species. Note identity of placental and monocyte tissue factor.

FIG. 23 is a tabulation of molecular masses (Da) of TF Proteins

FIG. 24 is a graphical view of factor VIIa titrations of recombinant Tf (rTf) 1-263, rTf 1-242 and human placental Tf (all at 0.1 nM) in the absence of lipid.

FIG. 25 is a graphical view of thrombin generation in synthetic plasma with platelets (2×10⁸/ml). Notice monocyte in situ and monocyte purified.

FIG. 26A,B are various views of Protein S (PS) preparations; FIG. 26A PS shows inhibition data and an SDS-PAGE analysis (inset) and FIG. 26B shows light scattering measurements of the rate of depolymerization of PS. Open squares: commercial PS and closed squares: in-house PS.

FIG. 27 is tabulation of the sets of equations describing the mechanisms of action of each of the anticoagulants used in the study described in Example 2 and which are useable in the simulating methodology of FIG. 3.

FIG. 28 is a tabulation of the rate constants controlling the identified bimolecular interactions and the dosing ranges recommended for prophylactic and therapeutic applications as described in connection with Example 2.

FIG. 29 is a tabulation of anticoagulation in contact pathway Inhibited blood.

FIGS. 30-A-D are various graphical views of thrombin as a function of time for showing the efficacy of AT dependent anticoagulants during the onset of Tf-initiated thrombin generation. Anticoagulants were incorporated into reactions prior to the introduction of 5 pM Tf reagent. The resulting time courses of thrombin formation are presented. FIG. 30A-UFH computational: (♦), control; (Δ), 0.015 U/mL; (), 0.05 U/mL. FIG. 30B—UFH empirical: (♦), control; (), 0.05 U/mL; (▪), 0.07 U/mL. FIG. 30C-Fpx computational: (♦), control; (□), 0.125 μM; (), 0.25 μM; (Δ), 0.5 μM. FIG. 30D-Fpx empirical: (), control; (□), 0.125 μM; (), 0.25 μM; (▴), 1 μM. Predicted thrombin concentrations are given at 1 min intervals to match empirical sampling. Thrombin values in the empirical control resupply time course (♦) represent mean±SD (n=5).

FIGS. 31A-D are various graphical views for showing efficacy of AT independent anticoagulants during the onset of Tf-initiated thrombin generation. Anticoagulants were incorporated into reactions prior to the introduction of 5 pM Tf reagent. The resulting time courses of thrombin formation are presented. FIG. 31A—C921-78 computational: (♦), control; (▪), 1 nM; (), 10 nM. FIG. 31B—C921-78 empirical: (♦), control; (▪), 1 nM; (), 10 nM. FIG. 31C—DAPA computational: (♦), control; (Δ), 10 μM; (◯), 20 μM; (□), 100 μM. FIG. 31D—DAPA empirical: (♦), control; (◯), 20 μM; (□), 100 μM. In the presence of DAPA, thrombin (IIa) generation is presented as levels of IIa=DAPA. Predicted thrombin concentrations are given at 1 min intervals to match empirical sampling. Thrombin values in the empirical control resupply time course (♦) represent mean±SD (n=5).

FIGS. 32A-D are various graphical views for showing efficacy of AT independent anticoagulants during resupply. Reactions initiated with 5 pM Tf reagent were resupplied after 20 min by the addition of an equal volume of starting material with or without anticoagulants, but no additional Tf. The resulting time courses of thrombin formation are presented. Thrombin levels for the final 5 min of the Tf-initiated episode are also shown (⋄). FIG. 32A-UFH computational: (♦), control; (□), 0.25 U/mL; (▴), 0.5 U/mL; (◯), 5.9 U/mL. FIG. 32B—UFH empirical: (♦), control; (□), 0.25 U/mL; (▴), 0.5 U/mL. FIG. 32C-Fpx computational: (♦), control; (), 0.25 μM; (▴), 1 μM; (▪), 5 μM. FIG. 32D-Fpx empirical: (♦), control; (), 0.25 μM; (A), 1 μM; (▪), 5 μM. Predicted thrombin concentrations are given at 1 min intervals to match empirical sampling. Thrombin values in the empirical control resupply time course (♦) represent mean±SD (n=16), with the SD less than 4% of each time point value.

FIGS. 33A-D are various graphical views for showing efficacy of AT independent anticoagulants during resupply. Reactions initiated with 5 pM Tf reagent were resupplied after 20 min by the addition of an equal volume of starting material with or without anticoagulants, but no additional Tf. The resulting time courses of thrombin formation are presented. Thrombin levels for the final 5 min of the Tf-initiated episode are also shown (⋄). FIG. 33A—C921-78 computational: (♦), control; (▪), 1 nM; (), 10 nM; (♦), 40 nM; (◯), 160 nM. FIG. 33B—C921-78 empirical: (♦), control; (▴), 40 nM; (◯), 160 nM. FIG. 33C-DAPA computational: (♦), control; (Δ), 10 μM; (□), 100 μM. FIG. 33D-DAPA empirical: (♦), control; (Δ), 10 μM; (□), 100 μM. In the presence of DAPA, thrombin (IIa) generation is presented as levels of IIa=DAPA. Thrombin values in the empirical control resupply time course (♦) represent mean±SD (n=16), with the SD less than 4% of each time point value.

FIGS. 34 A,B are various views showing time courses of prothrombin activation after resupply. Proteome mixtures initiated with 5 pM Tf reagent were resupplied after 20 min by the addition of an equal volume of starting material with or without anticoagulants. FIG. 34A (an immunoblot)—control resupply (no anticoagulant). Lanes (a-c, 100 ng/standard), II, prothrombin (Mr=72,000); pre1, prethrombin 1 (Mr=50,000, residues 156-579); F1.2A (Mr=47,000, residues 1-320); F1.2 (Mr=40,000, residues 1-271); B chain, α-thrombin B chain (Mr=30,000, residues 321-579). Lane (d) is the 20 min sample taken immediately prior to resupply. FIG. 34B (composite Western blot or a composite of 5 immunoblots) shows the time courses of prothrombin antigen levels after resupply in the presence of each inhibitor. Prothrombin levels are shown over the first 15 min after resupply with UFH, Fpx, DAPA or C921-78.

FIG. 35 is a graphical view of TAT as a function of time for showing suppression of the resupply response in whole blood: Fpx vs. C921-78. A time course of TAT formation after the addition of 5 pM Tf reagent to contact pathway inhibited blood is shown (♦). A parallel set of blood aliquots exposed to 5 pM Tf reagent at the same time was incubated for 20 min and then resupplied with CTI blood (⋄), CTI blood+1 μM fondaparinux (final) (◯) or CTI blood+160 nM C921-78 (final) (A) and the reactions then quenched at the indicated times. TAT levels are expressed as the total picomoles TAT versus time (min) to normalize for volume change. Also shown TAT levels at 20 min in Tf-initiated reactions when C921-78: (), 1 nM; (♦), 10 nM or Fpx: (), 0.25 μM; (▪), 0.5 μM; (X), 1 μM were present from the onset. Clot times (CT): are presented for: a, control; b, 1 μM Fpx; c, 160 nM C921-78.

FIGS. 36 A, B are graphical view of thrombin as a function of time for showing computational assessments of a hypothetical direct fIXa inhibitor. FIG. 36A—The hypothetical inhibitor was incorporated into reactions prior to initiation with 5 μM Tf. The existing time courses of thrombin formation are: (♦), control; (◯), 1 nM; (▴), 10 nM. FIG. 36B—The hypothetical inhibitor was incorporated into the resupply mixtures. The resulting time courses of thrombin formation are: (♦), control; (◯), 40 nM; (▴), 160 nM. Thrombin levels for the final 5 min of the Tf-initiated episode are also shown (⋄).

FIG. 37 is a graphical view of thrombin as a function of time showing the thrombosis history for the protein (C) population.

FIG. 38 is a tabulation of the evaluated population in which the model was run with and without a Protein C pathway.

FIGS. 39A,B are graphical views of thrombin versus time of Factor VIII titration using numerical simulations at 5 pM TF stimulus. A FVIII titration of 0%, 1%, 5%, 10%, 25%, 40% and 100% FVIII are illustrated for individuals having their other factor levels (FII, FVII, FIX, FX, AT and TFPI) set to the high range of normal (FIG. 39A) or tot low range of normal (FIG. 39B).

FIGS. 40A-C are graphical views of thrombin versus time showing the influence of a 100-fold rise in estradiol on simulated thrombin generation. FIG. 40 A shows IVF subjects (n=7) before and after gonadotropin stimulation and a theoretical control (physiologic mean). The data are shown as mean and SD in gray (+SD for after therapy and −SD for before stimulation). FIG. 40B shows IVF subjects at basal, after stimulation and after stimulation levels of FVIII, AT and TFPI set back to basal levels (shown as the mean and −SD in gray). FIG. 40C shows IVF subjects after stimulation (mean±SD), a theoretical control (physiologic mean), and after stimulation levels of FVIII, AT and TFPI set back to theoretical control levels (mean−SD in gray).

### DESCRIPTION OF THE PREFERRED EMBODIMENT

As discussed more fully hereinbelow, the present invention relates to methodologies for modeling a molecular pathway and for predicting a plurality of steps in a cascade or pathway. Such modeling also can be used to predict effects of candidate compounds (e.g., drugs) on the plurality of steps in a cascade or pathway. Generally, as used herein, a “pathway” refers to a plurality of reactions (e.g., chemical reactions, binding reactions, and the like), such as those which are involved in biochemical, cellular, physiological, and/or pathophysiological processes. A pathway may interconnect with, and/or be regulated by one or more other pathways. Preferably, the one or more other pathways also can be modeled as a series of chemical reactions and/or binding reactions.

In one aspect, a pathway being modeled comprises “pathway components” (e.g., such as enzymes, substrates, cofactors, ligands, receptors, ions, signaling molecules, transport molecules, DNA, RNA, ribosomes, transcription factors, translation factors, and the like) and “interaction values” which are associated with the components (e.g., concentrations, rate constants, binding affinities, dissociation rates, catalysis rates, transfer rates, rates of synthesis, etc.) in a relational database according to the invention. Preferably, the methodology of the present invention is embodied in a computer program product or computer program (hereinafter reference to computer program product or program product shall be understood to also include a computer program) that manipulates the values to generate a series of time-dependent concentration profiles for any/all reactants/components in a pathway over any time frame of interest. More preferably, the methodology and computer program product implements a problem solver, such as a Runge-Kutta problem solver, to perform these computations.

The methodology embodied in the program product also can be used to model the effects of additional objects introduced into the system (e.g., compounds, such as drugs, other pathway molecules, other pathways), the effects of modifications of existing components (e.g., protein modifications, mutations, etc.), and/or the effects of altering “system values” such as temperature, pH, and the like. In preferred aspects, the program product is used to model a pathway that is individualized for a particular patient, e.g., such as a patient suffering from a disease or predisposition to a disease.

Preferred practice involves using a methodology embodied in the program product to model the extrinsic coagulation system (particularly stoichiometric anticoagulants) and to accommodate the formation, expression, and propagation of the vitamin K dependent procoagulant complexes. In one embodiment, the model includes at least about 34 differential equations and at least about 42 rate constants.

In one aspect, the present invention provides a method for modeling the effect of a candidate compound on the pathway being modeled including the blood coagulation pathway. For example, the effect of a compound, such as an inhibitor of clotting (e.g., an antithrombotic agent) or an accelerator of clotting (e.g., a therapeutic agent for treating a hemorrhagic disease) is evaluated using the methodology of the present invention, preferably embodied in a program product, to identify particular step(s) of the pathway that would be affected by introduction of the compound. In this way, the methodology and program product embodying same is used to design an appropriate treatment strategy for a pathology which qualitatively or quantitatively alters steps of the pathway, i.e., providing a user of the program with the ability to select the parameters of a drug (e.g., range of binding affinities for a particular blood clotting factor, range of effects on catalysis, etc.) that would at least partially restore step(s) of the pathway to normal or be used as a prophylactic to reduce hemostatic risk. Candidate compounds then are screened to identify those that fit selected criteria defined by the model.

In a preferred aspect, the methodologies of the present invention including those embodied in a program product, model one or more pathways that is/are individualized for a particular patient. For example, patient-specific concentrations of pro- and anti-coagulants are inputted into the computer program model such as through a user interface. Assuming that rate constants are unchanged, the computer program model generates time-dependent concentration profiles for reactants unique to that patient over a time frame of interest (e.g., during a period when symptoms are expressed or during a period when symptoms are not expressed or when the patient is exposed to a particular therapy). In an exemplary embodiment, the patient has a congenital or acquired condition which affects blood clotting or the model is utilized to predict or determine if the patient or subject is pre-disposed to for example, thrombosis. Such conditions include, but are not limited to, deficiencies in one or more of fibrinogen, Factors II, V, VII, VIII, IX, X, XI, and XII, well deficiencies in ATIII, plasminogen, protein C, protein S, etc; and conditions caused by exposure to agents such as heparin, coumadin, etc. Changes in rate constants themselves also can be modeled by empirically determining rate constants unique to a patient using methods routine in the art.

In particular aspects of the present inventions, the methodology is structured so that a limited array of coagulation factors, all of which are at levels considered clinically normal, can be integrated by a computational model to predict an individual's potential to generate thrombin and thus be related to that individual's hemostatic risk in a variety of settings. For example, acquired data indicates that hypothetical thrombin generation based upon coagulation factor composition can distinguish between ACS and CAD. Individuals within the ACS population generated significantly higher levels of thrombin at a 50% faster rate resulting in more thrombin over the time course of the TF-initiated coagulation reaction. These prothrombotic thrombin generation profiles of individuals with ACS appear to be driven primarily by collective alterations in factor VIII, antithrombin and prothrombin levels. These results suggest that a limited array of coagulation factor and inhibitor levels, all largely within the accepted normal range for these factors/levels, potentially contribute to the procoagulant phenotype in acute phases of CAD. This supports the concept that “vulnerable” circulating blood with prothrombotic alterations and a vulnerable plaque, a potential source of tissue factor (TF), may both play a role in the development of ACS.

The phenotypic blood composition at any time not only reflects ongoing systemic events (e.g. inflammation) but also genetic predisposition. When considered in the context of CAD, blood composition responds relatively rapidly to external influences compared to the overall progression of the vascular remodeling. In principal, these changes in blood composition can translate into either a more or less procoagulant state, which can be evaluated by methods which assess thrombin generation. The procoagulant phenotype observed in the ACS population derived from relatively small changes (between 10 and 30%) primarily in only three factors, and is either a consequence of the acute event (that is developed within the first 12 hours from the onset of chest pain) or predated the event by some time interval. In the ACS population, the procoagulant phenotype appears to depend primarily on the influence of AT, factor VIII and prothrombin upon thrombin generation. In general, alterations in the production or clearance rates of these factors could yield these changes. It should be noted that since two of the factors are elevated and one decreased, the alterations are not uniform. Overall, without being bound by any particular theory, in the present invention the integration of blood composition data into an assessment of thrombin generation potential discriminates between acute and stable CAD and also that a limited array of factors can be predictive.

It should be recognized that while the foregoing examples refer to ACS and CAD, the present invention is not limited to applying the modeling methodology for such applications. It is within the scope of the present invention for such modeling methodologies to be extended to include, but not limited to, the following areas of applicability are: hemophilia (A (fVIII), B (fIX), C (factor XI), factor VII and V deficiency), thrombophilia factor V Leiden and thrombosis Protein C deficiency, Antithrombin deficiency hyperprothrombinemia genetic mutations that effect analytes, In vitro fertilization, oral contraceptive use, genetic mutations that effect analyte levels, fibrinogenemias platelet disorders, coumadin therapy, anticoagulant therapy (unfractionated heparins, synthetic heparins, direct thrombin inhibitors, direct factor Xa inhibitors, factor VIIa inhibitors, factor IXa inhibitors, surgical procedures (pre and post operative) trauma, or resuscitative therapy coagulopathies associated with traumas. Also, the modeling methods of the present invention are useable for drug development as the modeling methodology output(s) is inclusive of all terms or factors in the system and thus can be used to target the drug design to make it effective for the targeted term or factor.

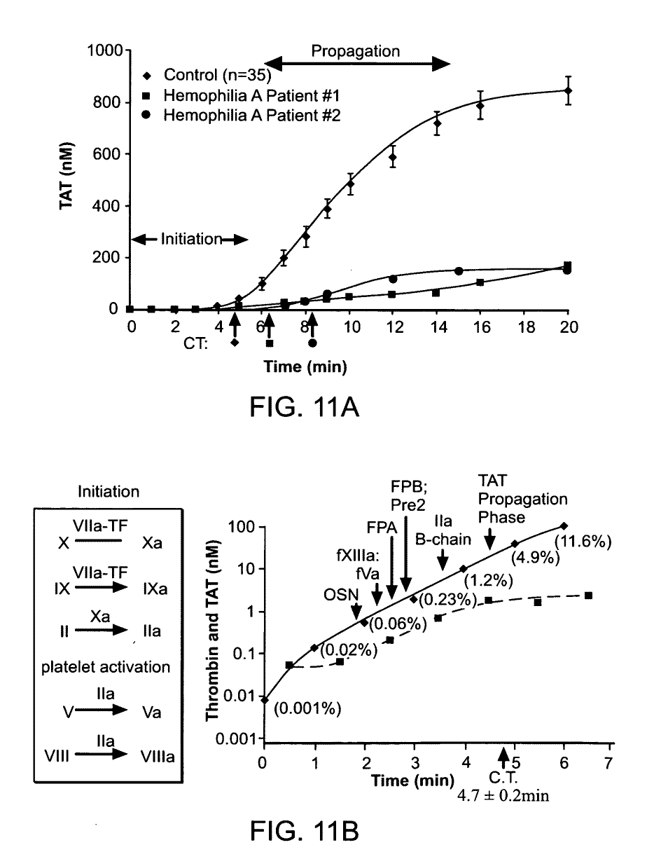

Prior to the last decade, most blood coagulation research could be divided between biochemical studies and studies involving clotting endpoints. The former were generally designed to provide good chemistry with concentrations and reaction conditions chosen to ensure high quality data but dynamics not necessarily relevant to biological conditions. More recently, a laboratory at the University of Vermont and those of Hemker and Hoffman-Monroe-Roberts have attempted to obtain quantitative biochemical data under conditions approximating the biological situation. At near biological conditions (whole blood), the display of thrombin generation following Tf triggering is shown in FIGS. 11A,B, which illustrates the generation of thrombin as thrombin-AT-III (TAT) (FIG. 11A) or TAT and active thrombin (FIG. 11B). Shortly following the addition of Tf, small amounts (mM) of thrombin are produced in an interval, which is defined operationally as the INITIATION PHASE of the reaction. Subsequently, the major bolus (>96%) of thrombin is produced during a PROPAGATION PHASE.

During the INITIATION PHASE, thrombin activates the substrates required to provide the catalysts (FIG. 11B), which generate the majority of the thrombin produced during the PROPAGATION PHASE of the reaction. Under normal circumstances, the rate-limiting component of prothrombinase complex formation and the ultimate generation of thrombin activity is the concentration of fXa. The activation of platelets and fV occur rapidly to produce surplus fVa and platelet membrane binding sites (FIG. 11B). However, under conditions of congenital deficiency, thrombocyto-penia, platelet pathology or antiplatelet pharma-cologic intervention, the Tf initiated reaction can become sensitive to fV or platelets.

As the reaction progresses, fXa generation by the more active “intrinsic factor Xase” complex exceeds that of the “extrinsic factor Xase” (FIG. 12). This numerical representation shows that while initially fXa is generated by fVIIa-Tf, this is superseded by fXa generated by fIXa-fVIIa, which generates most of the fXa. In the absence of fVIII or fIX, the “intrinsic factor Xase” cannot be assembled, thus no amplification of fXa generation occurs. This is the principal defect observed in hemophilia A and hemophilia B.

The presentation of a clot (“CT” in FIGS. 11A,B) depends on the generation of ˜2 nM active thrombin. Thus, at high Tf concentrations the robust generation of fXa by fVIIa-Tf completely masks the contribution of the fIXa-fVIIIa complex in clot endpoint assays. This is the case for the standard PT assay in which thromboplastin (Tf (˜10 nM) and phospholipid) is chosen to produce a clot time of 11-15 s. In FIGS. 11A,B, a concentration of 5 pM Tf was used, producing a clotting time of ˜5 min. In hemophilia A and B, at 5 pM Tf, the clotting time is somewhat prolonged, however, the major defect is associated with the absence of a PROPAGATION PHASE (FIG. 11A).

The combinations of intensities of activation and inhibition provide tight regulation of the hemostatic process, establishing reaction thres-holds, essentially leading to an “on/off” switch.

While the contributions of blood plasma and cellular components which control the coagulation response are substantial, the contribution of the vascular wall cannot be minimized. Ordinarily the vascular endothelium, in healthy individuals, provides an anticoagulant blood container, which is both stoichiometric and dynamic in its opposition to thrombin generation. However, during an inflammatory crisis or following the rupture of an atheroschlerotic lesion, the vascular surface itself can support a prothrombotic response.

Extravascular tissue surrounding the endothelial cell layer also provides connective tissue collagens, which interact with von Willebrand factor and platelets. This tissue is also a rich source of Tf. The third major contributor to clot formation is an altered flow state. As pointed out by Virchow, flow and/or stasis within the blood vessel supplies additional contributions to processes occurring at the site of a vascular lesion. Population studies have identified risk factors for venous thrombosis including PC, PS and AT-1 deficiencies; fV^{Leiden}, prothrombin 20210 gene mutation, high fibrinogen and fVIII, etc. However, heterozygous presentation or elevated levels of any of these components is not a signal for clinical intervention with an anticoagulant. Pharmacologic interventions that are used in VTE are principally Coumadin and heparinoids in various forms ranging from synthetic pentasacch-arides through fragmented heparinoids to conventional heparin. Anticoagulants on the horizon include protease inhibitors for fXa and fIXa. The thrombin inhibitor Hirulog (Angiomax) has seen increasing success in invasive cardiology. The oral thrombin inhibitor Ximelagatran looked promising but has not received FDA approval because of liver toxicity. On the platelet side, chronic interventions with aspirin and clopridogrel are common while both ReoPro and Integrilin have found roles in invasive cardiology. Statins may also play roles in suppressing thrombin generation.

The numerical modeling embodied in the present invention resulted from the realization that analysis of the kinetics of the formation and expression of this 5 component system (fXa, fVa, fI, phospholipid, Ca++), could not be accomplished using the traditional biochemical approaches of changing one variable at a time. Therefore, the numerical model embodied in the present invention was developed so as to embody the stoichiometry and enzymology of prothrombinase formation and catalytic function. Using this model, conditions were definded under which paradoxical situations might be observed such as inhibition by excess substrate, enzyme or membrane. With the aid of this computer model, conditions can be defined under which empirical laboratory tests can be performed to illustrate this paradoxical behavior. Empirical conformity to theory encouraged further modeling.

Subsequently, coincident with our ability to reconstruct the entire procoagulant sequence of the Tf pathway from purified components, a non-linear, highly coupled model was developed describing the processes leading from Tf to thrombin. By constructing an empirical procoagulant proteome, this model was tested in a recapitulated physical system. More recently, consistent with the development of appropriate empirical models describing the function of TFPI and AT-III, the numerical model was further developed to incorporate these stoichiometric inhibitors. Constructing an empirical recapitulation of the coagulation proteome incorporating these inhibitors, permitted tests which validated the model.

These numerical models are constructed using classical mechanistically based second order equations for reaction parameters. These ensembles of equations are then composed with rate constants that are either uniquely developed or are gleaned from the literature. The model of Tf-initiated blood coagulation is based upon the law of mass action in which a set of coupled, nonlinear differential equations govern the time evolution of protein concentrations. As indicated herein, the simulation model or simulator herein described employs a fourth order Runga-Kutta algorithm to evaluate this family of differential equations, generating a series of time-dependent concentration profiles for all reactants, intermediates, and products. The concentrations of the procoagulant factors (II, V, VII, VIIa, VIII, IX, X) and the anticoagulants (ATIII, TFPI) that are “exposed” to pM concentrations of Tf in the model can reflect mean physiological concentrations for each factor or actual values for an individual. Simulations can be generated in which rate constants are varied or in which competing or novel pathways are incorporated. Entering new reaction pathways does not require writing differential equations; the software program embodying the methodology of the present invention, based upon chemical notation, automatically generates the equations.

Computer models tested by synthetic coagulation proteome and whole blood experiments have been utilized in the design of therapeutic interventions in fVII deficiency, in hemophilias and in studies of the heterogeneity of the human healthy population. Hemophilia A and B replacement studies, and evaluations of hypothetical rfVIII molecules with improved stability have been accomplished. The utility of peptide inhibitors of APC as adjuvants for hemophilia therapy also has been evaluated. Illustrations of the applications of these models to fundamental biochemical and clinical studies are as follows.

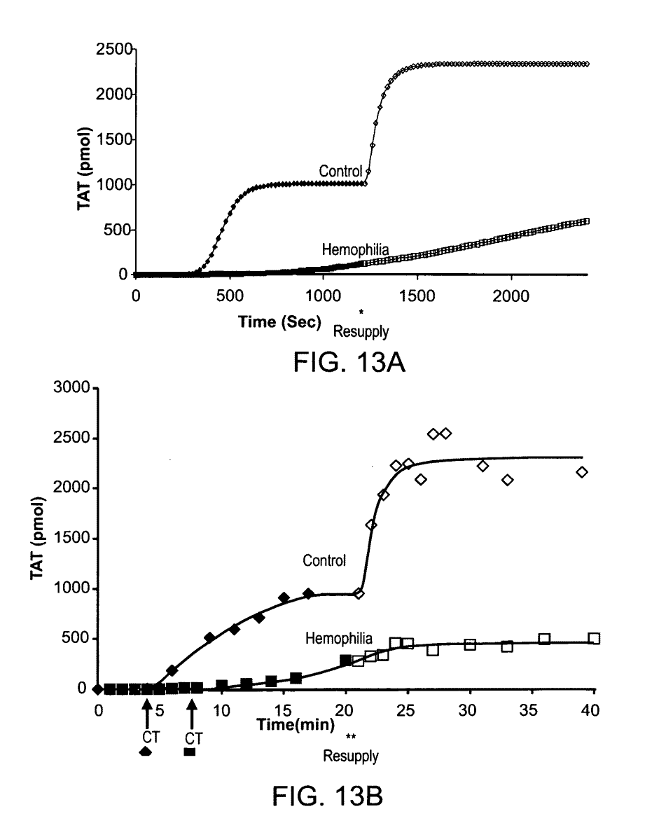

Experiments have been conducted simulating the resupply of blood components to a preexistent coagulation system by sequential “blood” additions to the reaction. These experiments have been conducted using the numerical model, the coagulation proteome and whole blood. FIG. 13A illustrates a computer model of hypothetical additions of blood to a reaction system, which, after Tf activation, achieved a quiescent level of thrombin formation (plateau of TAT). When a second supply of “blood” components (without additional Tf) is added, a rapid burst of thrombin generation (TAT) occurs. FIG. 13B illustrates an empirical study in which corn trypsin inhibitor (CTI) whole blood activated by the addition of Tf is allowed to become quiescent with respect to thrombin generation (TAT plateau see also FIG. 11A). When a subsequent aliquot of whole blood from the same donor (without Tf) is added, a response similar to that predicted by the numerical model is observed (FIG. 13A). If the influence of Tf is withdrawn either electronically (FIG. 13A) or immunochemically (α-Tf antibody) (FIG. 13B) just prior to the beginning of the propagation phase, there is no influence on the reaction; i.e. Tf is not required after the propagation phase has commenced. Also illustrated in both experiments is an evaluation of the equivalent experiment in hemophilia blood with a subsequent addition of fresh hemophilia blood (see also FIG. 11A).

The model embodied in the methodology of the present invention assumes unrestricted free diffusion in control of complex formation. This conclusion was reached by stop flow light scattering measurements of prothrombinase complex formation. It is noteworthy in both the numerical model and the blood system, which has clotted at 5 minutes, that no lag in thrombin generation is observed when fresh blood is added. Thus, in human whole blood, restricted diffusion does not appear to play a major role in thrombin generation, as has been hypothesized by some investigators.

In the absence of fVIII, no significant increase in thrombin generation occurs upon readdition, since the propagation phase is a consequence of fXa produced by fIXa-fVIIIa (FIG. 12). The resupply experiments as described, support the conclusion that the ultimate attenuation of the blood clotting reaction is controlled by flow cessation.

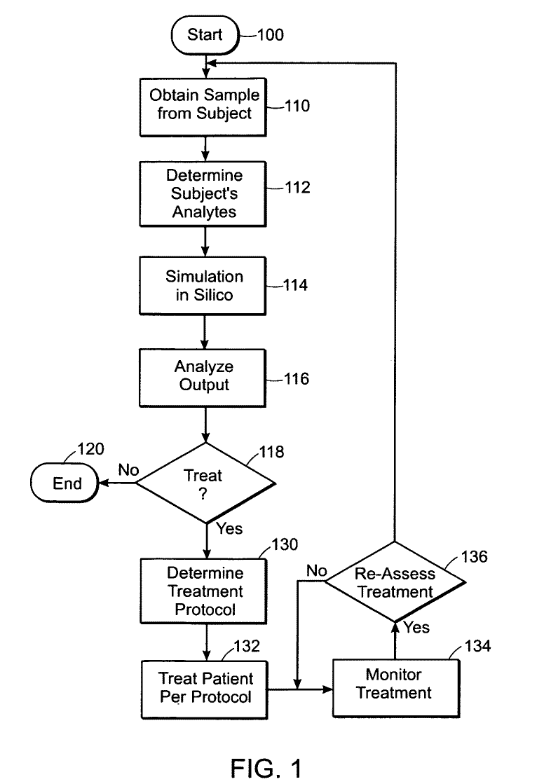

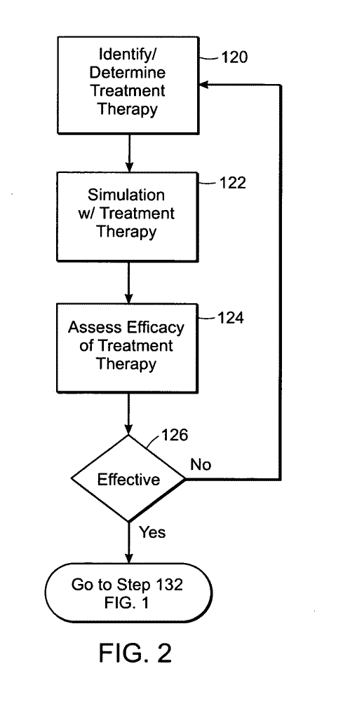

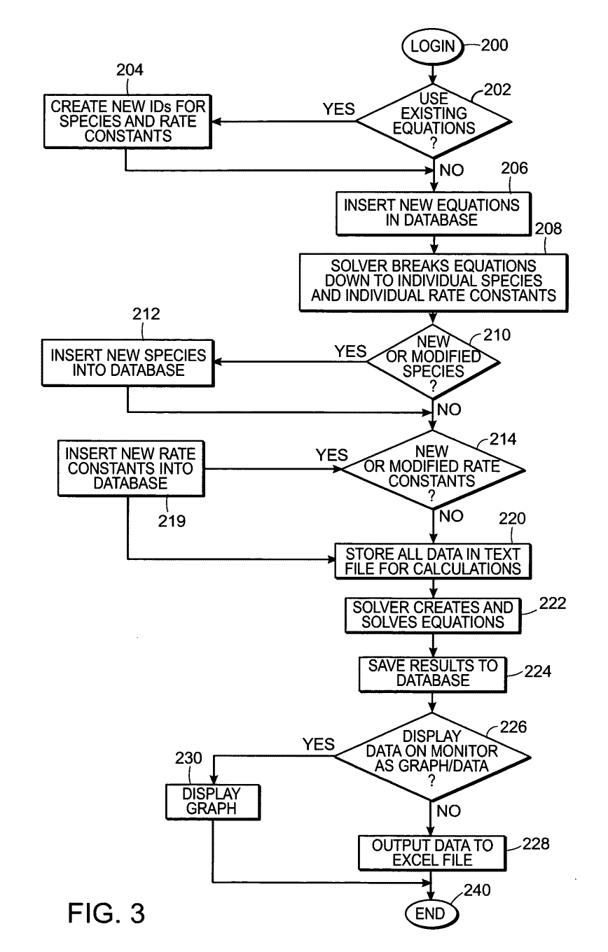

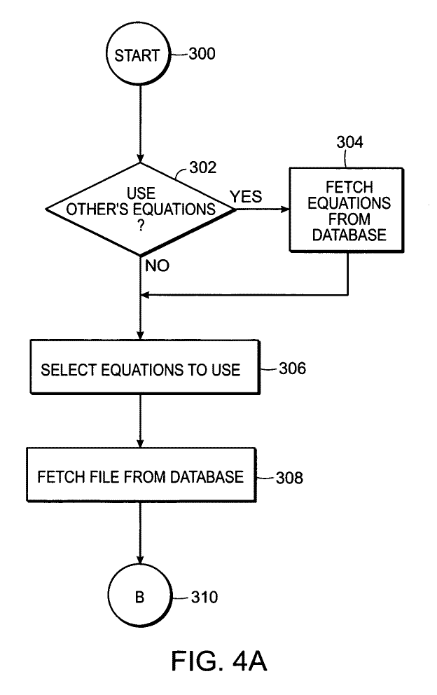

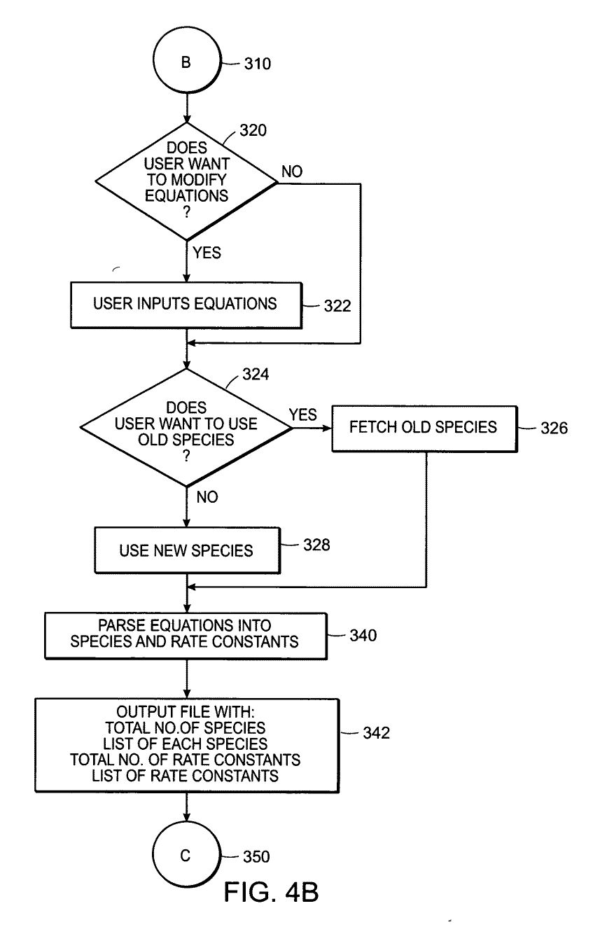

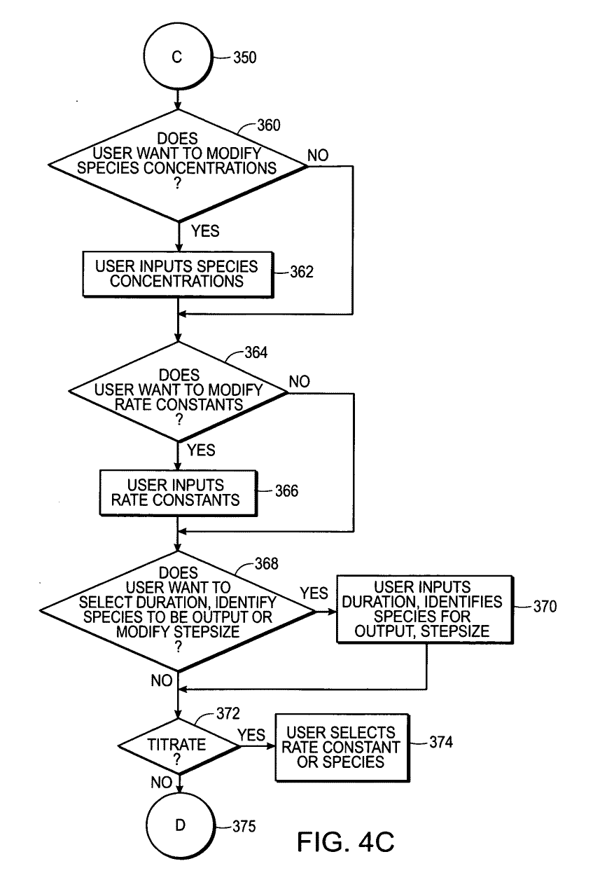

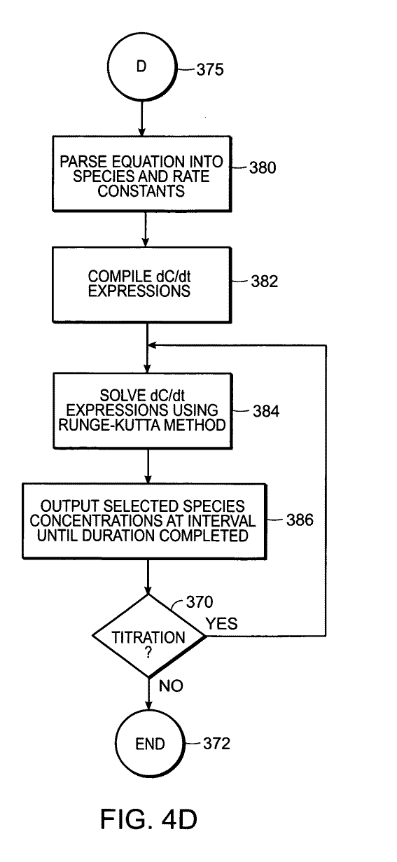

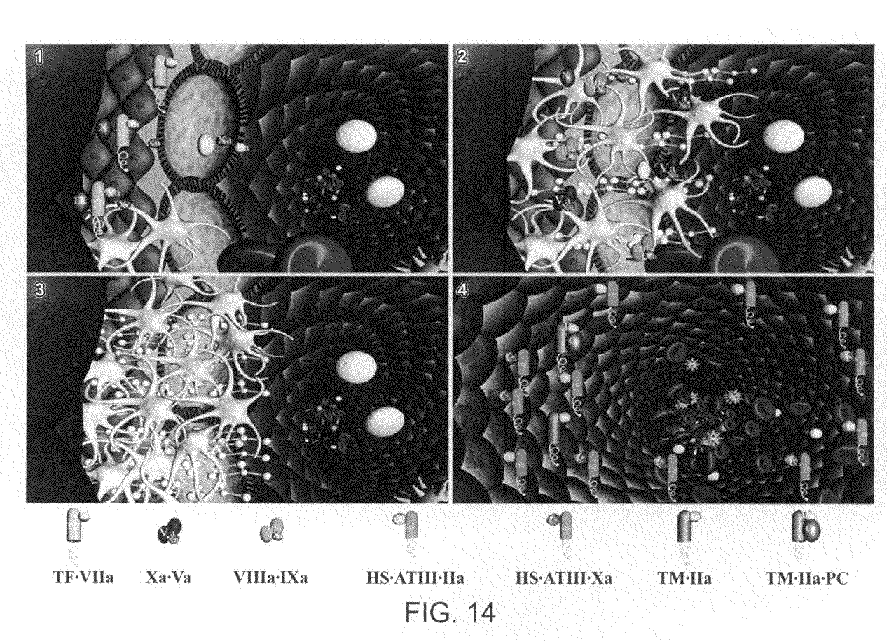

The reaction/flow control of bleeding is illustrated in FIG. 14, which hypothesizes a two compartment model for the regulation of Tf induced blood coagulation with flow. This model is illustrated in four cartoon panels. Perforation (FIG. 14-1) results in platelet adhesion, vascular Tf presentation and formation of an initial platelet-fibrin plug (FIG. 14-2). Additional reactants supplied under flow (FIG. 14-3) eventually plug the vascular defect. Active components still present in the plug provide uninhibited fIXa-fVIIIa and fXa-fVa which will rapidly initiate more thrombin-plug formation if exit flow continues. Downstream (FIG. 14-4), enzymes and cofactors escaping from the plug perforation reaction are captured by AT-III-heparan-proteoglycan complexes while cofactors are inactivated by the PC system.

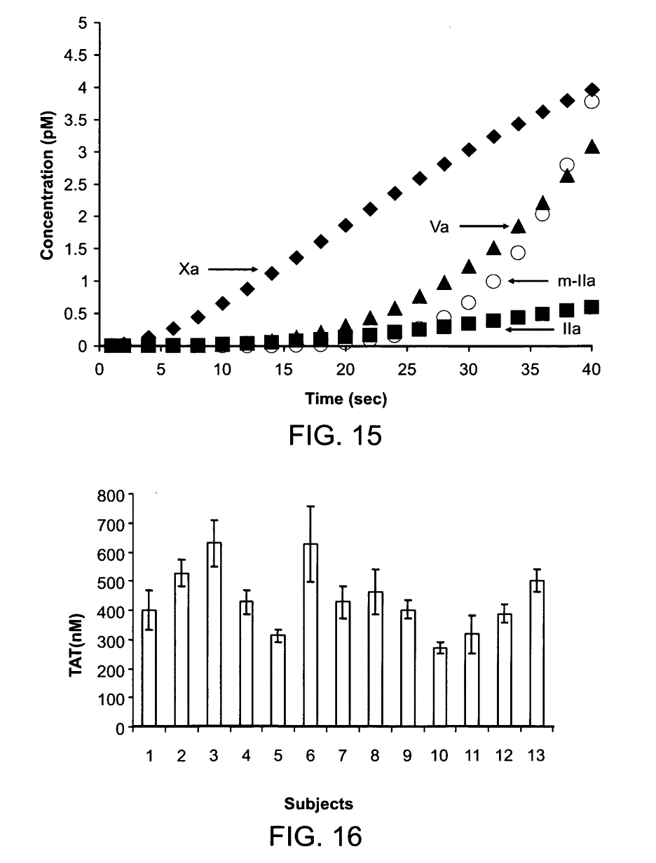

Early events of Tf initiated coagulation (FIG. 11B) involve the generation of the catalysts and cofactors, which then assemble into the pro-coagulant complexes responsible for most thrombin generation. These amplifying cycles take place at levels of thrombin frequently at the subnanomolar range. Femtomolar to picomolar enzyme concentrations lead to the generation of complex catalysts that are 10⁵-10⁷ times more efficient than the naked enzymes that carried out the initial catalytic events. The computer models describing these early events were instrumental in designing experiments and verifying conclusions because of the low concentrations of reactants and products. A display of these hypothetical early events predicted by one of these computer models is illustrated in FIG. 15.

When the data of FIG. 11A are evaluated on an individual basis, significant heterogeneity is observed. This heterogeneity of the phenotype with respect to thrombin generation was evaluated by constructing computer and empirical models which evaluated the influence of variations of coagulation protein concentrations over the “healthy” range (which typically extends from 50-150% of a mean value) on thrombin generation. The hypothesis that these computer and empirical studies suggested was evaluated in a six month clinical study of the ability of an individual's blood to respond to Tf. In these experiments, phlebotomy blood from 13 males was CTI inhibited and induced to react by the addition of 5 pM Tf. Following 20 minutes (corresponding to the plateau of thrombin generation, FIG. 11A), the reaction was quenched and TAT production evaluated (FIG. 16).

When challenged with the same Tf insult, significant differences in total thrombin production were observed from individual to individual while TAT generation within each individual's blood was fairly constant. Thus, thrombin generation approaches the status of a phenotypic property, consistent with empirical reconstructions and computer models.

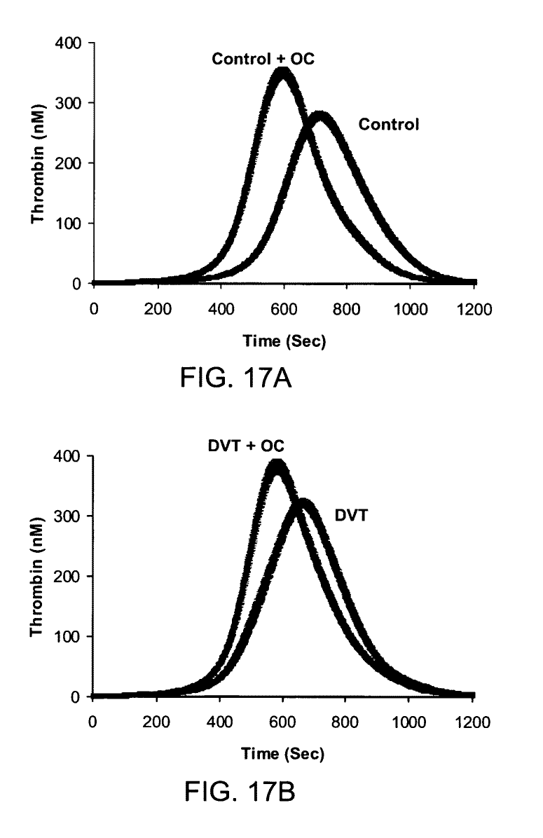

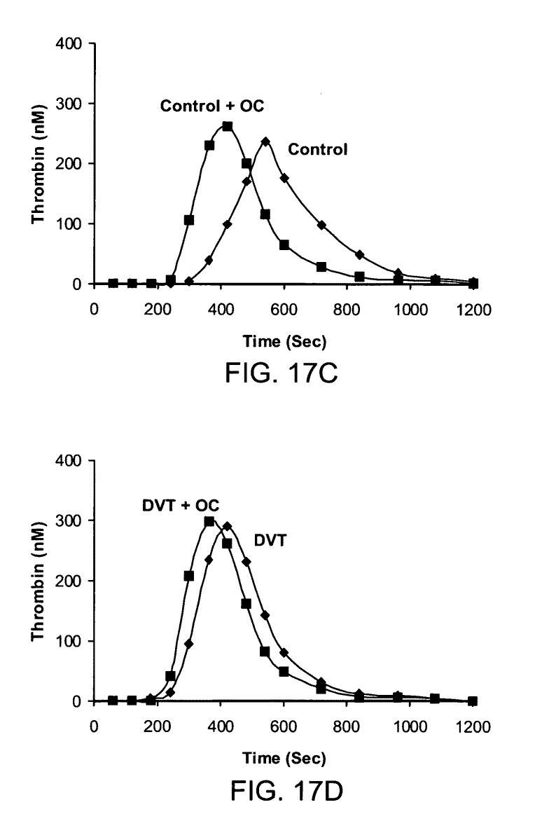

These observations were further expanded by numerical analyses of the controls and cases from the Leiden Thrombophilia Study (LETS). For the control population (472 individuals), significant variations in hypothetical thrombin generation with a Tf insult were observed. This variability was not explained by any one plasma factor. Simulations were performed independently for each individual (472) in which one of the eight factor levels was set to 100%, while the others were the actual values. The resulting >4000 thrombin generation profiles showed that under the influence of any one factor thrombin, generation varied by less than 9%. These data suggest that the overall composition dominates a healthy individual's propensity to form thrombin upon a Tf insult. In a paper studying the case population (472) from LETS, the risk associations for the maximum rate of thrombin generation obtained gave odds ratios of 3.9 in men, 2.1 in women and 2.9 in women on oral contraceptives (OC). OC use created extreme shifts in thrombin generation in both the control populations and OC women with prior thrombosis suggesting an interaction of OC use in cases with underlying prothrombotic abnormality (FIGS. 17A, B), consistent with the known risk of OC use.

A comparison of thrombin generation predicted from cohort plasma compositions using computer simulations and empirical simulations using the proteome at mean population factor concentrations show similarity (FIGS. 17 C,D). The coincidence of predicted and empirical results provides further evidence that the proteome is an appropriate candidate for deterministic modeling.

Venous thrombosis is most frequently associated with the accumulation of genetic-environmental alterations resulting in incomplete penetrance of any one polymorphism. In contrast, hemophilia A and hemophilia B are monogenic hemorrhagic defects clearly identified with risk with therapy available. However, even in hemophilia A, a variety of phenotypic expressions are seen. In collaboration with Dr. van den Berg of the van Creveldkliniek, Dutch National Hemophilia Treatment Center in Utrecht, N.L. 41, individuals with severe hemophilia A (less than 1% fVIII) were evaluated.

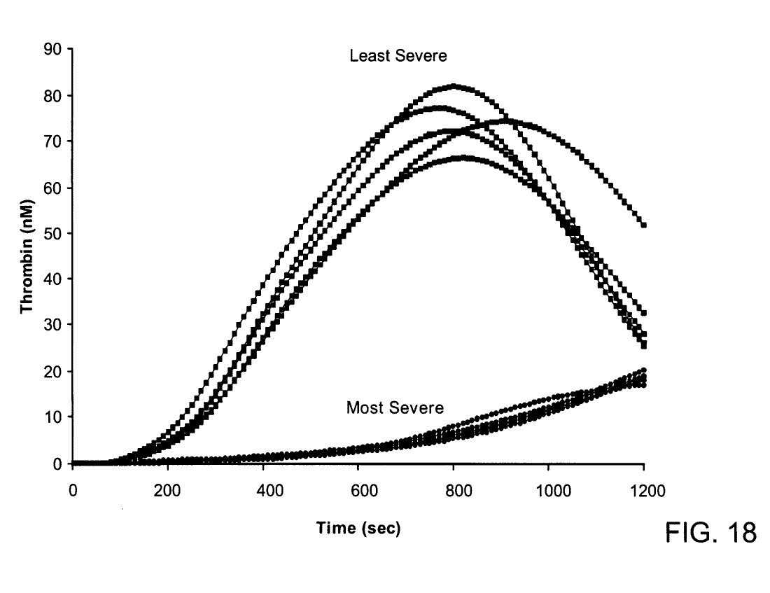

Based upon composition data, the hypothetical ability of these individuals to generate thrombin was calculated. The thrombin generation hypothetical constructs for 5 individuals at each extreme are shown in FIG. 18. Although all show impaired thrombin generation, a variable ability to generate thrombin with a Tf insult is observed. This variety implies that heterogeneity of one's blood composition can play a role in contributing to the variation in clinical severity in genetically severe hemophilia.

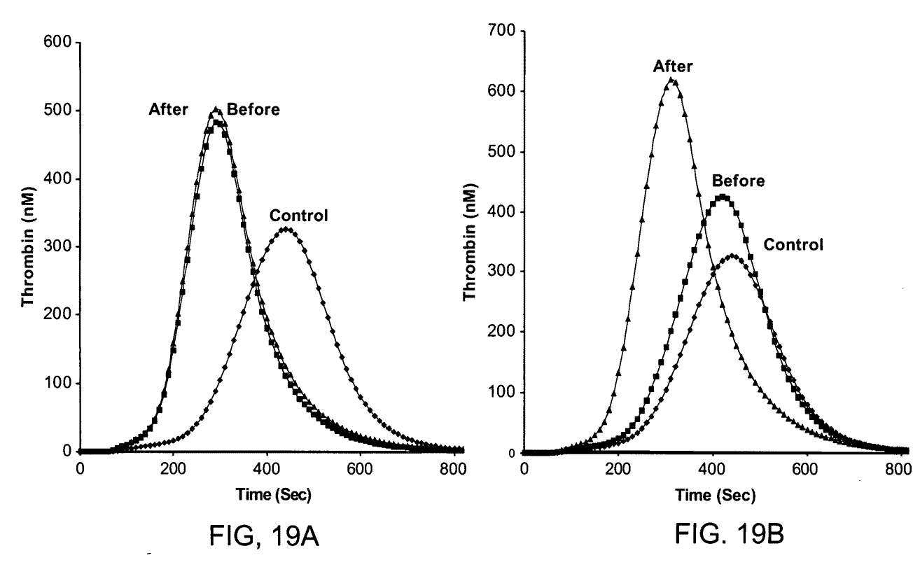

In a pilot study women who were undergoing In Vitro fertilization (IVF) were evaluated. These protocols involve the infusion of very large concentrations of hormone to induce ovulation. One of the downsides of this therapy is an increased risk for VTE. The plasma samples obtained from 5 women were evaluated during the course of this therapy and their coagulation protein complement was evaluated in our numerical models. There is shown in FIGS. 19 A,B graphical views for two individuals, one of whom shows little or no increased hypothetical thrombin generation following hormonal treatment (FIG. 19A) while the other shows massive increases in thrombin generation reflecting alterations in coagulation factor composition (FIG. 19B). It is inferred that the latter individual would be more susceptible to a thrombotic event in this therapeutic protocol.

The potential utilization of recombinant fVIIa (rfVIIa) for individuals with fVII deficiency was evaluated. This deficiency state, while rare in the general population (1 in 1 million), is more common in Quebec (heterozygous fVII deficiency 1 in 550). Since a natural or recombinant fVII therapeutic product was not available, the efficacy of utilizing rfVIIa using our model systems was evaluated. This study is important because fVII binds Tf and thus buffers IVIIa. Thus, in a fVII CRM (−) individual, the rfVIIa levels needed would be significantly lower than those required for an individual with hemophilia A. The numerical simulations supported this hypothesis and these data were largely replicated in proteome models and by reconstitution of fVII (−) blood in vitro and in vivo in fVII deficient individuals.

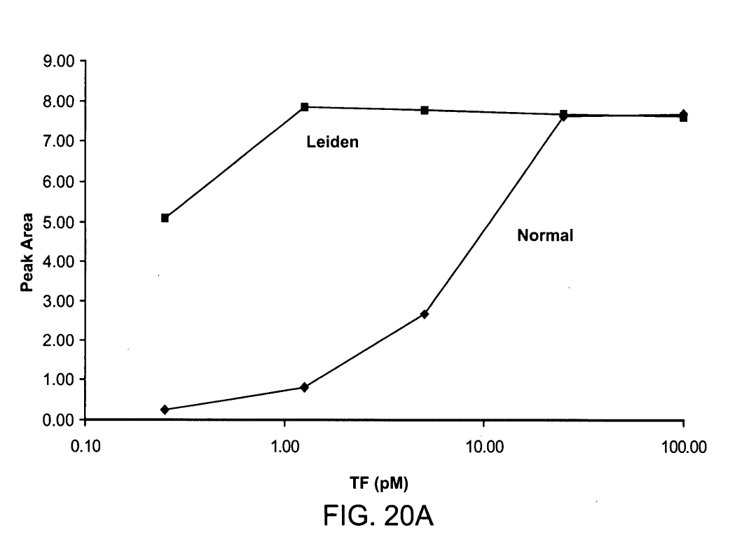

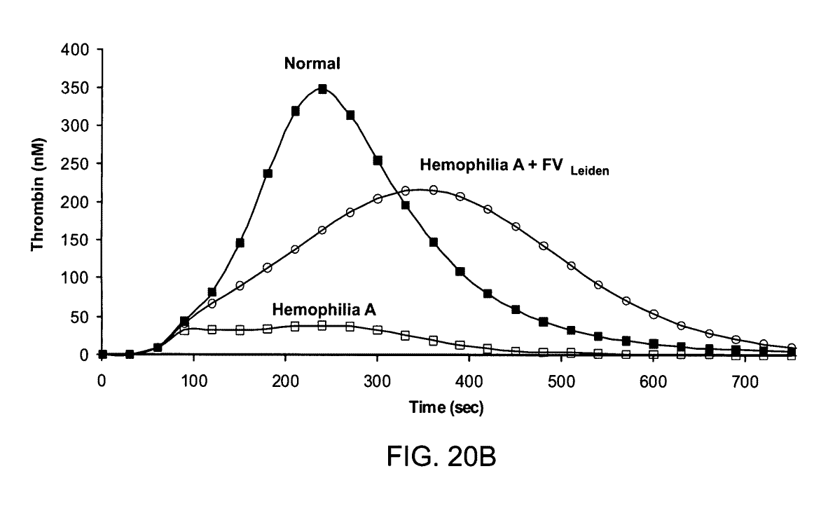

In regards to the APC system, the model and methodology were refined so that this includes elements of thrombin-Tm activation of PC and APC inactivation of fVa by virtue of cleavage at 306 and 506 in the peptide structure. Studies of the inactivation process of fVa supported by R01-HL34505 provides the mechanistic/quantitative data for this reaction and a global kinetic model for the loss of activity in the function of fVa following APC cleavage. Applications of the model fVa inactivation process are illustrated in FIGS. 20A,B.

FIG. 20A reflects the amount of thrombin which would be generated in homozygous fV^{Leiden} plasma as compared to normal as a function of Tf and is consistent with the pathology of this mutation. In FIG. 20B, an illustration of a hypothetical fVIII deficiency with coinheritance of fV^{Leiden}, a situation which has been hypothesized to be protective from hemophilia, is compared to normal and hemophilia with normal fV. The observations of FIG. 20B led to the development a collection of inhibitors of APC. These compounds yield increases in thrombin generation similar to those observed in FIG. 20.

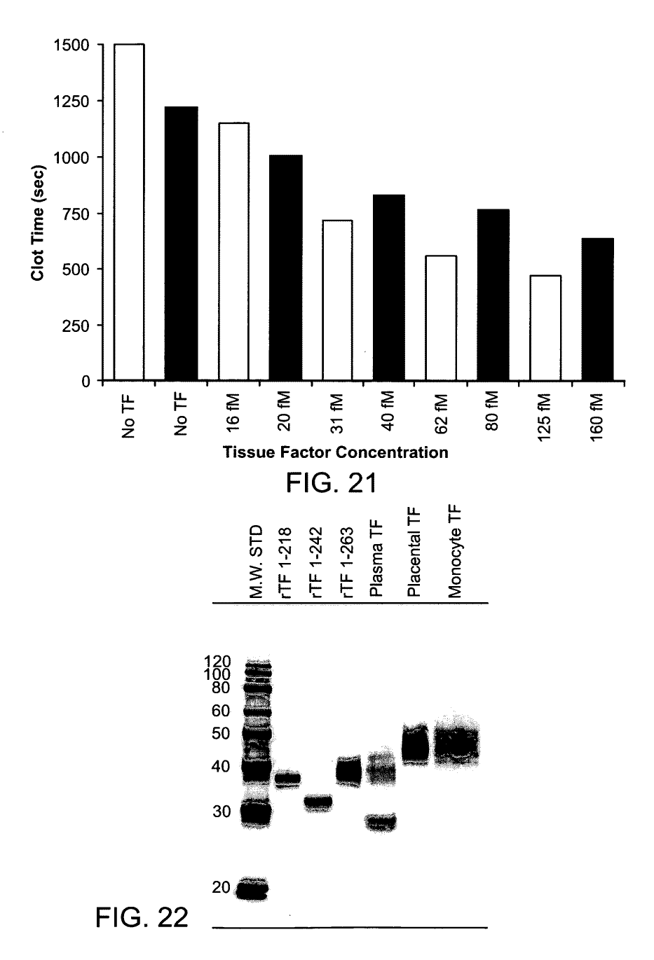

The report that active Tf circulated in soluble form in blood and the consequences of this potential paradigm shift in the initiation of the coagulation system response has led to significant effort being devoted to understanding natural Tf. Toward this end, a monoclonal antibody Tf isolation procedure from human placenta based upon acquisition of 11,000 vials of Thromborel S was developed. This permitted the isolation of approximately 30 mg of natural human placental Tf. Using a dual monoclonal immunoassay developed for Tf and a modified CTI-blood clotting assay, the concentration of functional and total Tf in whole blood was evaluated. The data shows that in healthy humans, the concentration of active Tf in whole (healthy) blood cannot exceed 20 μM (FIG. 21), while immunoassays of Tf in whole blood show a mean value of ≦2 pM Tf related antigen.

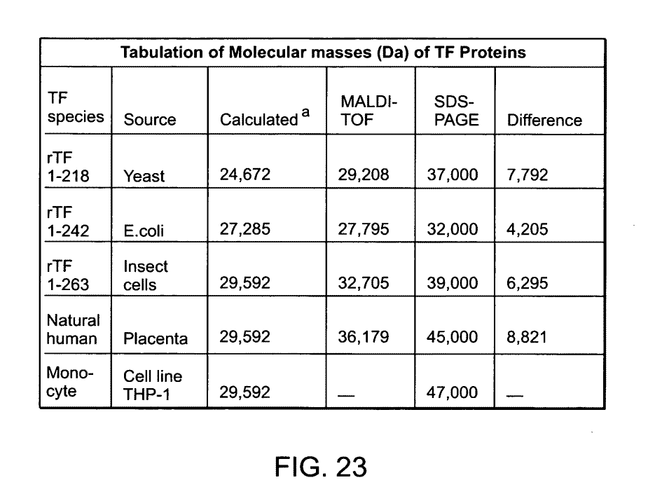

Tf also was isolated from THP-1 monocytes after stimulation with LPS. An immunoblot illustrating each of these natural and rTf products 1-218, 1-242 and 1-263 is shown in FIG. 22. A compilation of the molecular weights of each Tf is shown in the tabulation provided in FIG. 23 This tabulation displays the only report of fundamentally sound molecular weight analyses for Tf preparations based on MALDI-TOF mass spectrometry.

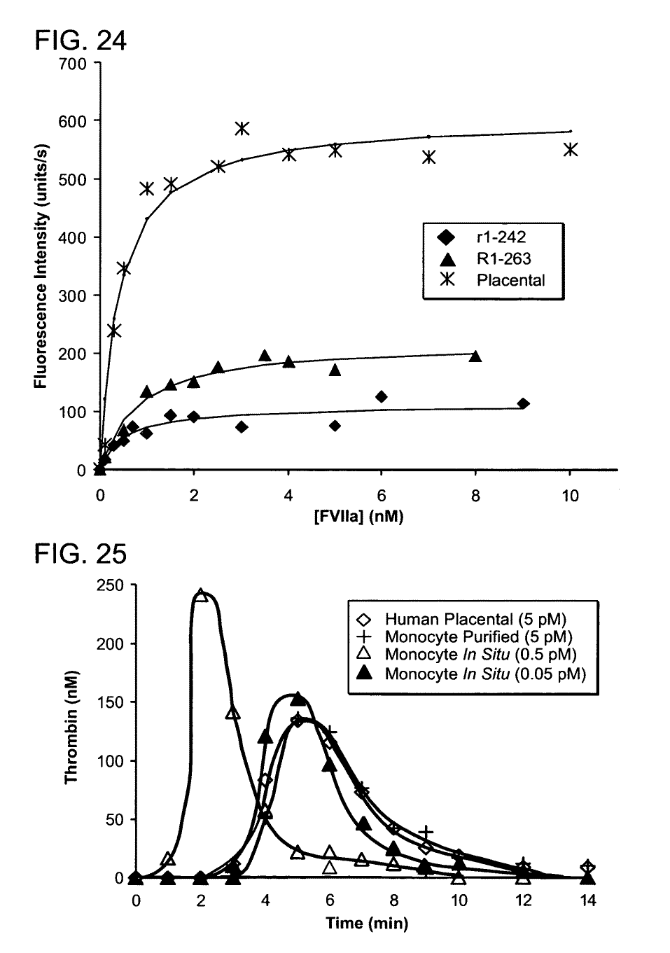

Reports of physical studies of Tf have made use of SDS-PAGE, amino acid analyses and presumed stoichiometry of binding. In a most recent review, a Tf molecular weight of 47 kDa is cited, introducing a 30% error for any experimental physical parameter regarding Tf and its interactions. Tf proteins are distinguished by functional activities in simple as well as in complex reaction systems (FIGS. 24, 25). Scatchard analysis shows that each Tf protein forms an equimolar complex with fVIIa. This 1:1 stoichiometry (based on the true molecular weights) validates assays used for the evaluation of Tf concentration and indicates that the observed difference in functional activity is not caused by erroneous estimates of Tf proteins.

In complex, placental Tf displays a lower K_{d} and a higher Vₘₐₓ than the recombinant proteins (FIG. 24). Monocyte Tf in situ, quantitated by immuno-chemistry, expresses significantly higher activity than monocyte Tf purified and reconstituted on phospholipids; purified monocyte Tf at 5 pM displays similar activity to monocyte Tf in situ at 0.05 pM. In the reconstituted coagulation platelet proteome, monocyte Tf displays ˜400× the activity associated with monocyte Tf reconstituted on synthetic membranes.

In conjunction with Osterud's laboratory, we observed that the Tf expression elicited by LPS stimulation is heterogeneous and phenotypically specific. This appears related to the ability of monocytes to express Tf on their surface rather than biosynthesize the protein following LPS stimulation. We obtain reproducible monocyte Tf expression with the THP-1 cells as a natural source of inducible Tf.

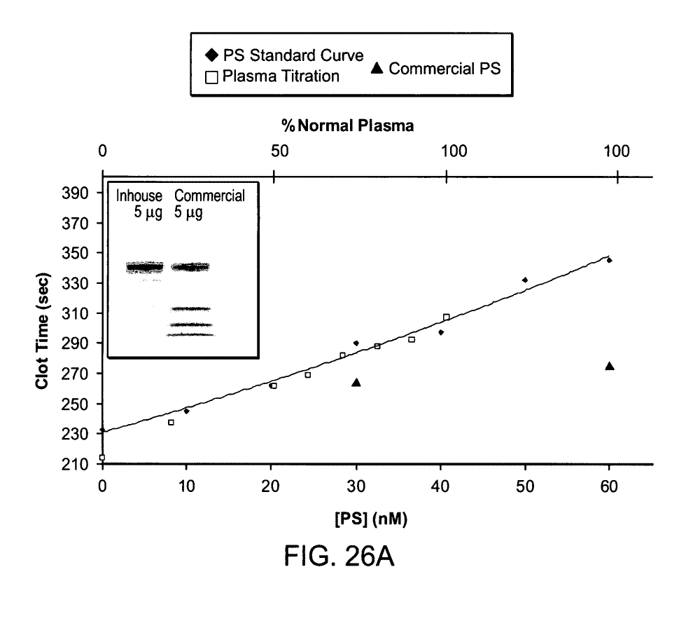

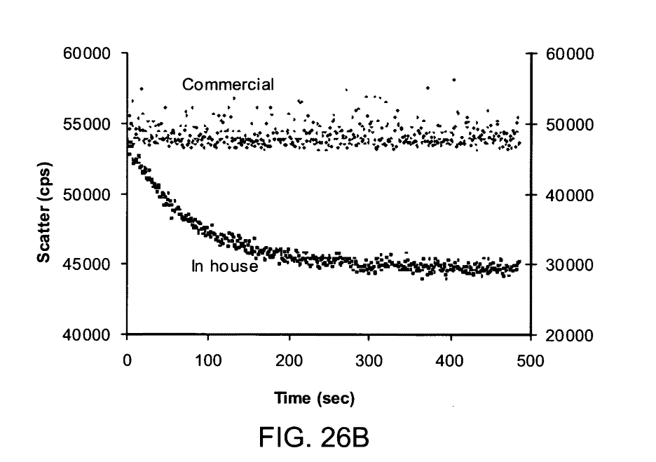

In regards to Protein S (PS) a Tf-based assay for PS in CTI-inhibited recalcified PS deficient plasma was developed. FIG. 26A shows clot inhibition data for purified recalcified PS and normal plasma added to PS deficient plasma. Dilutions of a normal plasma pool fall on this curve (open symbols). This titration indicates that normal plasma contains 40 nM free PS. The convergence of plasma dilution data with the purified protein curve lends support of the preservation of natural PS quality in the material we isolate. The rate of depolymerization of Ca⁺⁺ free PS upon addition of Ca⁺⁺ using light scattering (FIG. 26B) was evaluated. At 25° C., the half time for this reaction is approximately 100 s, thus the PS effect is significantly diminished in assays initiated in vitro in citrate plasma by the addition of Ca⁺⁺. A commercial preparation of PS (ERL) does not show this depolymerization behavior (FIG. 26B). The preparation also shows significant heterogeneity.

Referring now to FIG. 1 there is shown, a high level flow diagram illustrating a method according to the present invention for assessing hemostatic risk and also for minimizing risk by determining a treatment protocol. The process starts at Step 100 and thereafter the logic flow then continues to Step 110. At Step 110, a clinician or technician acquires a biologic sample from the subject or patient that is appropriate for use in determining the parameters necessary for the simulation in silico. In illustrative embodiments, the clinician or technician or other medical personnel withdraws one or more samples of blood from the patient or subject.

After obtaining the biologic sample, the logic flow continues to Step 114, where the subject's analytes are determined. Such determining includes measuring and quantifying each of the analytes. Thereafter, the logic flow continues to Step 104 in which a simulation of a biological process, for example a blood coagulation process, is performed such as described further herein. The simulation yields one or more outputs of parameters, for evaluation and analysis by the clinician or other medical personnel, Step 106.

In such analysis, the clinician compares the outputs to reference values or other criterion for determining if the outputs are symptomatic of a disease state or that the results indicate that the patient or subject is pre-disposed such that an undesirable medical event could occur in the future. For example, the results could indicate that the subject is likely susceptible to the occurrence of a clotting event or other hemostatic risk. If the clinician determines that treatment is not required or suggested (No, Step 118), then the evaluation process is concluded, Step 120.

If on the other hand, the clinician determines that the results or outputs are indicative of a need to initiate treatment so as to either treat a disease condition or to initiate a prophylactic treatment to reduce the risk to the patient or subject to minimize the hemostatic risk to the patient (Yes, Step 118), then the clinician would determine an appropriate treatment protocol, Step 130. For example, the results could indicate that the subject appears susceptible to undesirable blood clotting and thus the clinician could suggest a treatment protocol to be followed to minimize the risk of such clotting occurring in the future.

Thereafter, in any one of manners or techniques known to those skilled in the art and appropriate for the particular treatment protocol, the patient or subject would be treated, Step 132, and such treatment would be monitored by the clinician, Step 134, in any of a number of ways know to those skilled in the art and appropriate for the particular treatment protocol. The clinician, also would evaluate the efficacy of the treatment protocol from time to time to determine if the protocol is effective or if other protocols should be considered, Step 136.

If the clinician concludes that the treatment protocol is effective (No, Step 136), then the monitoring of the treatment protocol is continued, step 134. Thereafter process steps 134-136 are repeated until the clinician determines that the treatment protocol is not or no longer effective (or the treatment protocol is otherwise terminated). In the case where the clinician concludes that the treatment protocol is not or no longer effective (Yes, Step 136) then the process steps 110-132 are repeated to develop another treatment protocol.

As indicated herein, according to an aspect of the present invention the simulation methodology is utilized to optimize or arrive at a treatment protocol for the patient or subject. Now referring to FIG. 2 there is shown a high level flow diagram of such a process. It should be recognized that this process carries out the functionality contained in FIG. 1, Step 130.

Using knowledge available to those skilled in the art, and the outputs of the simulation, the clinician would identify an treatment therapy which would appear to be appropriate for the subject, step 120. Such a treatment therapy would include the intensity of the therapy. Also, in the cases where such therapy included the use of drugs, medicaments, replacement therapy or the like, this also would include the number of doses and dosage of such drugs, medicaments, replacement therapy or the like.

Using the determined subject's analytes based on the obtained biologic sample or based on a new biologic sample, a simulation is run with the treatment therapy as an input to the simulation process, Step 122. This simulation would be similar to that described herein such as for example, step 104 of FIG. 1. After the simulation is completed, the clinician evaluates the output to determine if the treatment therapy or treatment protocol is effective, Step 126. If the treatment plan or protocol is determined that to be effective (Yes, Step 126), then the process would return to FIG. 1, Step 132.

If the simulation results suggest that the planned treatment therapy is not effective (No, Step 126), then the process or logic flow would return to step 120. In this step the clinician would alter the treatment therapy, for example increase or decrease the dosage of the drug being administered and then repeat steps 122-126 of the process until a treatment therapy plan/protocol is arrived at. It should be recognized, that it also is within the scope of the present invention for a clinician to use this process to evaluate a number of possible different treatment therapies to determine which of the possible different treatment therapies is optimal.

The present invention is particularly advantageous, because the inputs to the simulation (based on the obtained biologic sample) are patient specific and thus the evaluation is not based solely on an assessment of the results in view of their normalcy to demographic or population range of values for healthy individuals. In contrast, most decisions using prior art techniques are usually based on an evaluation of a parameter with respect to the normal range of values for healthy individuals. A problem is assumed to exist when the parameter is out of the normal range. The present invention also is particularly advantageous because the simulation provides a mechanism by which the clinician can assess a subject's hemostatic risk before there is threat of the risk related event occurring. The present invention also is particularly advantageous, as it allows the clinician to simulate the treatment plan/protocol and assess its effectiveness before the subject or patient is exposed to the treatment plan.

Moreover, as the simulation methodology of the present invention can be used to assess the effectiveness of a given drug having certain characteristics or qualities to treat a given condition, the simulation methodology also is adaptable for use in drug development. Simply, one can input qualities related to a drug and target a given pathway for example. Thereafter, one assesses the effectiveness of a drug having that quality following such simulation. In the case where it is determined from the simulation that those drug qualities would be effective, then one can evaluate the pharmaceutical databases to identify a compound that exhibits the identified qualities. Thereafter, the efforts normal required to bring a drug to market can then be undertaken. Given the cost and time for bringing drugs to market, the ability to simulate and assess effectiveness before undertaken such efforts would be very advantageous as compared to the techniques currently used to identify compounds for targeting disease conditions (e.g., anti-coagulant drugs).

In regards to the simulating step (Step 104) of FIG. 1 and the stimulating step (step 122) of FIG. 2, reference should be made to FIGS. 3-4 and the following discussion. Reference also should be made to the published application for PCT Application No. PCT/US03/07379 and/or U.S. Patent Application Publication No. US2006/0015261 for further details and discussion relative to specifics of the methodology for simulating blood clotting and the use of this program for simulating the efficacy of such clotting with and without drug treatment. The following discussion regarding FIGS. 3-4 also is based on a related discussion provided in U.S. Patent Application Publication No. US2006/0015261.

The flow charts herein illustrate the structure or the logic of the present invention as embodied in computer program software for execution on a computer, digital processor or microprocessor. Those skilled in the art will appreciate that the flow charts illustrate the structures of the computer program code elements, including logic circuits on an integrated circuit, that function according to the present invention. As such, the present invention can be practiced by a machine component that renders the program code elements in a form that instructs a digital processing apparatus (e.g., computer, digital signal processor, AISC or the like) to perform a sequence of function step(s) corresponding to those shown in the flow diagrams. Also, and although not specifically stated in the following, it shall be understood that an application programs embodying the methodology of the present invention shall include code segments, instructions and criteria including data as well as audio and visual data, so the applications program can carry out the below described functions/methodology.

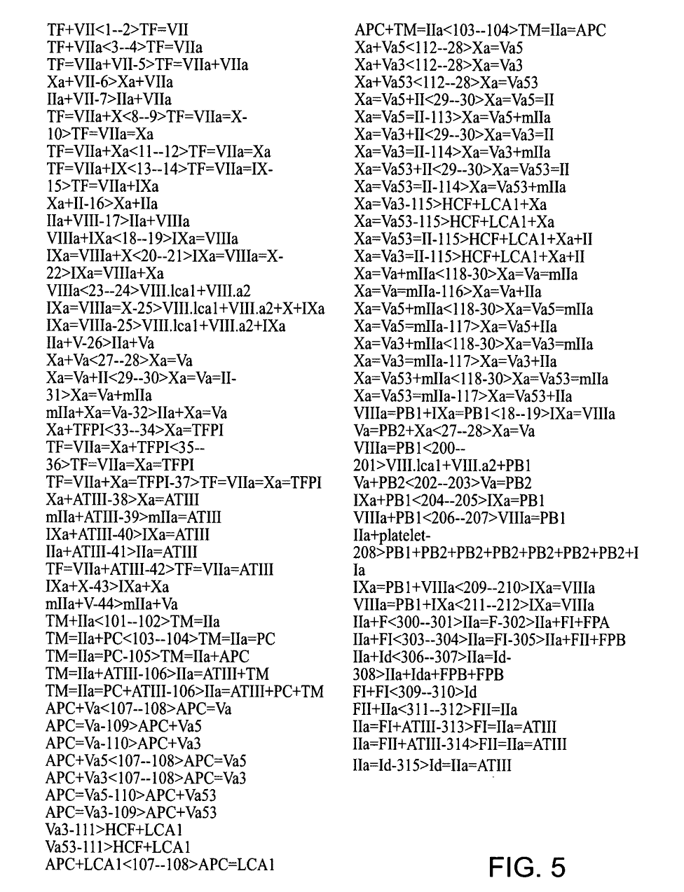

Referring now to FIG. 3, there is shown a block diagram representing a logic sequence of a methodology for simulating in silico so as to thereby assess hemostatic risk. Beginning at Step 200, a user logs in with a user name and password. Logic flow then continues at Step 202, where the user may select existing equations from a prior run of the program product. An exemplary listing of such equations is provided in FIG. 5; however, it shall be understood that the simulation carried out in accordance with the methodology of the present invention is not limited to these equations. It is within the scope of the present invention to include equations hereinafter developed that provide a further mechanisms for assessing some aspect of hemostatic risk.

If the user selects the existing equations (Yes, Step 202), then logic flow continues at Step 204, where the methodology embodied in the program creates new IDs for the species and rate constants. Logic flow then continues at Step 206. If the user does not select the existing equations (No, Step 202), then logic flow continues at Step 206, where the user inserts new equations into the database. Logic flow then continues at Step 208. In Step 208 a feature called solver (e.g., a subroutine, called the “solver”) breaks down the equations to individual species and rate constants. Logic flow then continues at Step 210, where the user selects new or old species. If the user selects the new species (Yes, Step 210), the logic flow continues at Step 212, where the new species is inserted into the database. Logic flow then continues at Step 214. If the user does not select new species but instead selects the old species (No, Step 210), the logic flow bypasses Step 212, and continues at Step 214.

At Step 214, the user selects new or old rate constants. If the user selects new rate constants (Yes, Step 214), logic flow continues at Step 219, where new rate constants are inserted into the database. The logic flow then continues at Step 220. If the user does not select new rate constants but instead selects the old rate constants (No, Step 214), the logic flow bypasses Step 219 and continues at Step 220.

At Step 220, all data used for the calculations is stored in a text file. At Step 222, the solver parses the text file created in Step 220, creates the corresponding equations, and solves them. The logic flow then continues at Step 224, where the results of the calculations are saved to the database.

The logic flow then continues at Step 226, where the user selects whether to display the data on the monitor as graphical data. If the user does not wish to display the data on the monitor (No, Step 226), the logic flow continues at Step 228, where the data is output to an Excel® formatted file. If the user wishes to display the data on the monitor as a graph (Yes, Step 226), the logic flow continues at Step 230, where the data is displayed. After display or output in Steps 230, 228, respectively, the logic flow terminates at Step 240.

Referring now to FIGS. 4A-D there are shown block diagrams representing the logic sequence of a methodology for the solver feature step of FIG. 3. The solver step of FIG. 3 is detailed more fully in the flowcharts of FIGS. 4 to 4D, and is the result of a mathematical model that was created to aid in the understanding of the blood coagulation system by modeling the kinetics of the enzyme linked systems. The solver step is limited to second order reactions, since its primary design is for use in biochemistry models.

As the collective understanding of the coagulation cascade matured, the model system evolved into ever greater complexity, requiring the simultaneous solution of systems of differential equations describing independent rate processes for over twenty (20) components. The ever increasing complexity of the system led to a bookkeeping problem, however. Given a single component, e.g., thrombin, which participates in five (5) separate processes related to its formation or consumption, alteration of any one of these processes through inclusion of alternative substrates or fates required changes in a number of independent dC/dt expressions. Thus, working by hand to re-write dC/dt expressions for numerical solutions became time consuming, and led to repeated periods of testing, wherein the simulations were run and rerun to insure that all equilibrium expressions were satisfactory and all dC/dt expressions included the necessary components. To overcome the inevitable human errors introduced into this process, a software package was developed that would take mathematical equations, develop the dC/dt expressions, and solve the system of equations at defined time points. With the continuous increase in CPU speed, and the evolution of the World Wide Web as a universal programming interface, a solver system was created that was universally applicable to all kinetic systems, and was then tailored to the chemistry of blood.

The equations used in the software program of FIGS. 4A to AD were written in the following format:

A+B<1−2>AB (1)

AB−3>A+C (2)

In the software, these equations were used to derive the fundamental equilibrium dC/dt expressions using the rules described here within:

$$\begin{matrix}\frac{d A}{d t} = - [ A ] [ B ] {k}_{2} + [ AB ] ( {k}_{1} + {k}_{3} ) & ( 3 ) \\ \frac{d B}{d t} = - [ A ] [ B ] {k}_{2} + [ AB ] {k}_{1} & ( 4 ) \\ \frac{d AB}{d t} = [ A ] [ B ] {k}_{2} - [ AB ] ( {k}_{1} + {k}_{3} ) & ( 5 ) \\ \frac{d C}{d t} = [ AB ] {k}_{3} & ( 6 )\end{matrix}$$

The methodology embodied in the software utilizes the format of equations (1-2) to generate expressions (3-6), thus eliminating human error often introduced when preparing these expressions by hand. The software prompts the user for initial concentrations and rate constant values. These values are then used to model the linked system and generate anticipated time dependent concentration values for each species at user specified intervals. The software performs rapidly, and, in one practical embodiment, generated time dependent concentration values for a system utilizing twenty-five (25) species tracked for five (5) minutes at one (1) second intervals in approximately fifteen (15) seconds.

Most chemical kinetic problems pose a significant difficulty for numerical analysis because the functions that describe the decay or appearance of a specific species contain the variable species concentration on each side of the expression, as well as multiple species on the right hand side (RHS). Further mathematical difficulties result from the necessity of including three separate dC/dt expressions to account for a single species. For example, species A in expressions 3-5:

$$\begin{matrix}\frac{d A}{d t} = - [ A ] [ B ] {k}_{2} + [ AB ] ( {k}_{1} + {k}_{3} ) & ( 3 ) \\ \frac{d B}{d t} = - [ A ] [ B ] {k}_{2} + [ AB ] {k}_{1} & ( 4 ) \\ \frac{d AB}{d t} = [ A ] [ B ] {k}_{2} - [ AB ] ( {k}_{1} + {k}_{3} ) & ( 5 )\end{matrix}$$

In these expressions, [A] appears on both the left and right side. It will be appreciated that dA/dt was explicitly written as d[A]/dt, while the RHS contains the unique species [A], [B], and [AB]. It will also be appreciated that each expression defined a separate component uniquely affecting dA/dt. By contrast, the first order rate problem encountered in radioactive decay calculations was as follows:

$$\begin{matrix}{}_{86}^{222} Rn - 1 > {}_{86}^{218} Po & ( 7 ) \\ \frac{{d}_{86}^{222} Rn}{d t} = - [ {}_{86}^{222} Rn ] {k}_{1} & ( 8 ) \\ \frac{{d}_{86}^{218} Po}{d t} = [ {}_{86}^{222} Rn ] {k}_{1} & ( 9 )\end{matrix}$$

In the above example, the expression for

$$\frac{{d}^{222} Rn}{d t}$$

is explicitly solvable and could have been written as:

$$Ln ( \frac{No}{N} ) = {k}_{1} t \qquad ( 10 )$$

where equation (10) is the result of explicit differentiation of the rate equation (7), yielding a simple exponential problem. Due to the nonlinear character of expressions (3-6), no such explicit solution is available. It will be appreciated that an infinite Taylor series expansion could have been utilized to indicate solutions to the differential expressions (3-6), and that computational algorithms based upon the Taylor series could have been utilized to describe the next step of a function y through its course:

$${y}_{n + 1} = {y}_{n} + \frac{d y}{d x} {\vert}_{n} \frac{Δ x}{1 !} + \frac{{d}^{2}}{d {x}^{2}} {\vert}_{n} \frac{Δ {x}^{2}}{2 !} + \frac{{d}^{3}}{d {x}^{3}} {\vert}_{n} \frac{Δ {x}^{3}}{3 !} + … + \qquad ( 11 )$$

Thus, if the instantaneous derivatives of a function y were known at any point, the value at points on either side could be evaluated through successive approximations using higher order terms. However, explicit solutions to ordinary differential equations are sparse for nonlinear systems. If an approximation/adaptation of the Taylor Series (11) is used, known as the explicit fourth order Runge-Kutta method, an approximate solution to the generalized initial value problem (12-13) is:

$$\begin{matrix}\frac{\vec{d y}}{d t} = \vec{f} ( t , \vec{y} ) \\ where & ( 12 ) \\ \vec{y} ( t = {t}_{0} ) = {\vec{y}}_{0} & ( 13 )\end{matrix}$$

Expressions (3-6) fell into the generalized description of initial value problems for which expressions (12-13) described, and approximate solutions could be found, using the Runge-Kutta algorithm. Known ordinary differential equations (ODE) analysis procedures for such systems included the Taylor method of order 4, the double precision 4th order Runge-Kutta solver, FORTRAN 77 (implemented in the preferred embodiment), as well as Hindmarsh's LSODE solver. The classical or 4th order Runge-Kutta algorithm utilizes the following approach to accelerate its rate of convergence:

$$\begin{matrix}{y}_{n + 1} - {y}_{n} = \frac{Δ t}{6} ( {k}_{1} + 2 {k}_{2} + 2 {k}_{3} + {k}_{4} ) \\ where & ( 14 ) \\ {k}_{1} = \frac{d y}{d t} {\vert}_{{t}_{0} , {y}_{0}} & ( 15 ) \\ {k}_{2} = \frac{d y}{d t} {\vert}_{{t}_{0} + \frac{Δ t}{2} , {y}_{0} + \frac{Δ {tk}_{1}}{2}} & ( 16 ) \\ {k}_{3} = \frac{d y}{d t} {\vert}_{{t}_{0} + \frac{Δ t}{2} , {y}_{0} + \frac{Δ {tk}_{2}}{2}} & ( 17 ) \\ {k}_{4} = \frac{d y}{d t} {\vert}_{{t}_{0} + Δ t , {y}_{0} + Δ {tk}_{3}} & ( 18 )\end{matrix}$$

Hindmarsh's LSODE was based on Backward Differentiation Formula (BDF) methods, mostly using 3rd order polynomials, but took control of the step size, and thus results in a more efficient computation. A reasonably accurate solution for Runge-Kutta could have been obtained for many functions, provided a sufficiently small step size in time, dt, was utilized.

One important, but necessary, condition for the evaluation of the set of differential expressions (3-6) is that the initial values concentration, in this case, be known and that the rate constants are defined appropriately. The methodology and software embodying same utilize equations (1-2) to determine the number of unique reactants, products, and rate constants. These unique species are then presented in a web interface with a text box requiring user entry of the values for the initial concentration of each species and each rate constant. It will be appreciated that, in the case of the coagulation model, most values for the initial concentration of each species were zero.

Since the Runge-Kutta method utilizes defined step intervals (in kinetics, this interval was time) to approach an experimental solution, the success of this method is largely dependent upon a careful choice of stepsize. The stepsize value should be optimized appropriately in order to maximize computational utility and experimental value. The use of large (greater than one (1) second) intervals was found to result in computational instability, resulting in the return of zero or NaN (not a number, i.e., infinite) values for the concentration of a few or most species of interest. Routine use of 0.01 second intervals resulted in computational stability for the coagulation simulations, and yielded values identical to similar type solvers running on diverse computer systems with diverse compilers, such as R10K Silicon Graphics Inc. (SGI) Octanes, and PII, PIII, and PPC machines running the Linux operating system. This approach required a large number of computational cycles, as the number of iterative calculations required in the solution of any equation system had to be non-linearly proportional to the order of the calculation. In one practical embodiment, the software program embodying the methodology was run with a start of 0.01 seconds, with the stepsize then decreased by a factor of ten. The results were then compared. If both were identical, it was not necessary to reduce the stepsize further. If computational instability was apparent, the stepsize was decreased again, and the iteration was repeated.

It will be appreciated that there was one further consideration regarding stepsize and data representation. The methodology embodied in the program product requests a duration interval from the user, which did not affect the computational algorithm. The software program calculates the species concentrations every tenth (0.1) second. Thus, a twenty (20) minute simulation generates 12,000 data points for each species. In the case of twenty (20) species, this results in 240,000 data points. Therefore, by default, the software generated data points at one (1) second intervals. It will be further appreciated that no round off error was introduced, as this method simply defined the data points to be output.

The modeling embodied in the software program, in accordance with the present invention, defines a rigorous system of inputting chemical equations in a manner that allows the computer to model the relationships among reactants, and products with their rate constants. The program also is designed such that reversible equilibrium expressions could be input as a single line, i.e., there was no need independently to define the forward and reverse reactions using separate lines. In illustrative embodiments, limits imposed upon the scripting language include:

1. Species names are limited to 20 characters in length and can contain any character string with the exception of the +, >, <, and − characters without spaces. The equation parser is case sensitive, i.e., a distinction is made between capital letters and lowercase letters. The characters >, <, and − are reserved to denote the direction of reaction progress and rate constant identity.

2. The total number of unique species in any system is limited to 1000.

3. The total number of rate constants is limited to 1000.

4. The description of rate constants are numeric integers of value less than 100. This is for the rate constant description, and not its value.

5. The maximum number of species that can be included in one side, e.g., on the RHS, of a single expression, is 5.

6. There is a limit of 70 characters per line in total. Accordingly, economy in notation is important for the system. It is to be recalled that the equation compiler is case sensitive, so that IIa and iia are considered distinct species.

The following are examples of irreversible and reversible systems of equations that assisted in a complete understanding of the scripting notation used in the software program. In general, the following text objects were used to describe forward reactions, reverse reactions, and equilibrium expressions.

−1> Reaction proceeds from left to right, as written, according to the rate constant 1.

<2—Reaction proceeds from right to left, as written, according to the rate constant 2.

<1−2> The reaction is an equilibrium expression proceeding left, according to the rate constant 1, and right, according to the rate constant 2.

The use of these text objects is shown below:

Irreversible reaction: A+B−1>D

Irreversible reaction: D<1−A+B

Equilibrium Expression A+B<2−1>C−3>D

When these reactions are entered, the computer executing the software program embodying the methodology of the present invention, requests initial values for the species A, B, C, D, and then for the rate constants 1, 2, and 3.

Turning now to FIG. 4A, the logic sequence for the solver step begins at step 300, and proceeds to a decision step, Step 302, where the user is interrogated as to whether to use another user's equations. If the user Wishes to use another user's equations (Yes, Step 302), the logic flow continues at Step 304, where the software program fetches a file containing the equations from the database, where they were saved from a previous run. If the user does not wish to use another's equations (No, Step 304), the logic flow continues at Step 306, where the user selects from the equations to be used for a run. At Step 308, the software program fetches the file from the database with the selected equations. The logic flow then continues at Step 310, or “B”.

Referring now to FIG. 4B, the logic flow continues at Step 310 or “B”, and then proceeds to step 320, where the software program interrogates the user as to whether the user wishes to modify the selected equations. If the user wishes to modify the selected equations (Yes, Step 320) the logic flow continues at Step 322, where the user inputs the modified equations. The logic flow then continues at Step 324. If the user does not wish to modify the equations (No, Step 320), the logic flow continues at Step 324.

At step 324, the software program interrogates the user as to whether the user wishes to user old species. If the user wishes to use old species (Yes, Step 324), the logic flow continues at step 326, where the program fetches the old species from the database. The logic flow then continues after Step 328. If the user wishes to use new species from Step 324 (No), the logic flow continues at Step 328, where the new species are used. At Step 340, the solver parses the equations into species and rate constants. The logic flow then continues at Step 342, where the program creates an output file with the total number of species, a list of all species, with one species on each line, the total number of rate constants, and a list of rate constants, with one rate constant on each line. The logic flow then continues at Step 350, or “C”.

Turning to FIG. 4C, the logic flow begins at Step 350 or “C”, and then proceeds to Step 360, where the software program interrogates the user as to whether the user wants to modify the species concentrations. If the user wishes to modify the species concentrations (Yes, Step 360), the logic flow continues at Step 362. Thereafter or in the case the user does not wish to modify the species concentrations (No, Step 360), the logic flow continues at Step 364. At Step 364, the software program interrogates the user as to whether the user wishes to modify the rate constants. If the user wishes to modify the rate constants (Yes, Step 364), the logic flow continues at Step 366, where the user inputs the rate constants. Thereafter or in the case the user does not wish to modify the rate constants (No, Step 364), the logic flow continues at Step 368.

At step 368, the software program interrogates the user as to whether the user wishes to select the duration, to identify the species to be output, or to modify the stepsize. If the user desires to select the duration, to identify the species to be output, or to modify the stepsize (Yes, Step 368), the logic flow continues at Step 370, where the user inputs the duration, the species to be identified on output, and the modified stepsize. The logic flow then continues at Step 372. If the user chooses not to make any selections at Step 368, the logic flow continues at Step 372, where the software program interrogates the user as to whether a titration is desired. If the user desires a titration (Yes, Step 372), the logic flow continues at Step 374, where the user selects the rate constants or species for the titration. The user also selects modify, high/low (150%, 100%, 50% of value entered previously) or user defined for the titration. The logic flow then continues at Step 375, or “D”. If the user does not select titration (No, Step 372), the logic flow continues at Step 375.

Referring to FIG. 4D, the logic flow begins at Step 375 or “D”, and proceeds to Step 380. At Step 380, the solver parses the equations into the species and rate constants. The logic flow then continues at Step 382, where the program compiles the dC/dt expressions. At Step 384, the software program solves the dC/dt expressions using the Runge-Kutta method as described herein and in US. Patent Publication US 2006/0015261. The software program is then caused to output, at Step 386, a file with the selected species concentrations at the selected interval until the selected duration is completed. The logic flow then continues at Step 370, where it is determined if the user selected a titration. If the user selected a titration (Yes, Step 370), the logic flow returns to Step 384 and Steps 384 and 386 are repeated until the titration is completed. If no titration was selected (No, Step 370), the logic flow ends at Step 372.

### Example 1

### Introduction

Coronary artery disease (CAD) is the leading cause of death for both men and women in the United States each year. Atherosclerotic lesions within the coronary arterial system are subject to mechanical and hemodynamic forces that can trigger disruption of atherosclerotic plaques [Lee R T, Kamin R D. Vascular mechanics for the cardiologist. J Am Coll Cardiol 1994; 23: 1289-95; Maclsaac A l, Thomas J D, Topol E J. Toward the quiescent coronary plaque. J Am Coll Cardiol 1993; 22: 1228-41]. Thrombus formation after plaque rupture is the trigger for abrupt coronary artery occlusion and subsequent acute coronary syndrome (ACS) [Falk E, Shah P K, Fuster V. Coronary plaque disruption. Circulation 1995; 92: 657-71].

The rupturing or erosion of atheromatotic plaque in the coronary artery exposes blood to extravascular tissue factor (TF), a membrane bound glycoprotein. Circulating factor VIIa (FVIIa) combines with the newly exposed TF and activates the zymogens FIX and FX to their respective serine protease, FIXa and FXa (for review see [Mann K G, Brummel K, Butenas S. What is all that thrombinfor? J Thromb Haemost 2003; 1: 1504-14]). The procoagulant intrinsic and prothrombinase complexes are subsequently formed and ultimately result in prothrombin activation to thrombin. Thrombin catalyzes the transformation of soluble fibrinogen molecules to fibrin, potently activates platelets, and amplifies its own generation by activating the plasma procofactors FV and FVIII and the zymogen FXI. Thrombin's procoagulant activity is regulated by dynamic (protein C) and stoichiometric [antithrombin (AT) and tissue factor pathway inhibitor (TFPI)] inhibitory pathways.\

Consistent with this mechanism increased circulating levels of thrombin and its markers characterize ACS [Falk E, Shah P K, Fuster V Coronary plaque disruption. Circulation 1995; 92: 657-71]. Prothrombin fragment 1+2, present at 1-2 nM, is roughly 2-fold higher than observed in stable angina patients, reaching maximum values in subjects displaying both ST segment elevation myocardial infarction (STEM I) and markedly elevated cardiac troponin, a marker of myocardial necrosis [Hoffmeister H M, Ehlers R, Buttcher E, Steinmetz A, Kazmaier S. Helber U, Szabo S, Beyer M E, Seipel L. Relationship between minor myocardial damage and inflammatory acute-phase reaction in acute coronary syndromes. J Thromb Thrombolysis 2003; 15: 33-9; Merlini P A, Bauer K A, Oltrona L, Ardissino D, Cattaneo M, Belli C, Mannucci P M, Rosenberg R D. Persistent activation of coagulation mechanism in unstable angina and myocardial infarction. Circulation 1994; 90: 61-8].

Elevated fibrinopeptide A, another indicator of thrombin activity during the early phase of myocardial infarction, has been shown to be an independent predictor of cardiac mortality [Li Y H, Teng J K, Tsai W C, Tsai L M, Lin L J, Guo H R, Chen J H. Prognostic significance of elevated hemostatic markers in patients with acute myocardial infarction. J Am Coll Cardiol 1999; 33: 1543-8]. Therapies directed at reducing thrombin generation, using antiplatelet and anticoagulant regimens have become the mainstays of treatment for both ACS and CAD [Harrington R A, Becker R C, Ezekowitz M, Meade T W, O'Connor C M, Vorchheimer D A, Guyatt G H. Antithrombotic therapy for coronary artery disease: the Seventh ACCP Conference on Antithrombotic and Thrombolysic Therapy. Chest 2004; 126: 5135-48S].

Numerous in vitro studies have shown that, under normal conditions, the generation of thrombin during TF-initiated coagulation displays a characteristic pattern [Brummel K E, Paradis S G, Butenas S, Mann K G. Thrombin functions during tissue factor-induced blood coagulation. Blood 2002; 100: 14852; Hemker H C. Begum S. Phenotyping the clotting system. Thromh Haemost 2000; 84: 747-51; Butenas S, van't Veer C, Mann K G. ‘Normal’ thrombin generation. Blood 1999; 94: 2169-78; 12 Sorensen B, Johansen P, Christiansen K, Woelke M, Ingerslev J. Whole blood coagulation thromboelastographic profiles employing minimal tissue factor activation. J Thromb Haemost 2003; 1: 551-8].

It also has been shown that features of this pattern differ from individual to individual and that some of these differences derive from variations, within the normal range, in the plasma concentrations of key coagulation factors [Butenas S, van't Veer C, Mann K G. ‘Normal’ thrombin generation. Blood 1999; 94: 2169-78; Brummel K E, Paradis S G, Branda R E, Mann K G. Oral anticoagulation thresholds. Circulation 2001; 104: 2311-7; Brummel-Ziedins K E, Pouliot R L, Mann K G. Thrombin generation: phenotypic quantitation. J Thromh Haemost 2004; 2: 281-8; Brummel-Ziedins K, Whelihan M E, Ziedins E G, Mann K G. The resuscitative fluid you choose may potentiate bleeding. J Trauma 2006; 61: 1350-8]. Recently, we have extended these empirical observations concerning the individualized nature of thrombin formation using a computational model that uses the coagulation factor concentrations of individuals to predict their course of thrombin generation [Hockin M F, Jones K C, Everse S J, Mann K G. A model for the stoichiometric regulation of blood coagulation. J Biol Chem 2002; 277: 18322-33].

For example, in the Leiden Thrombophilia Study (LETS) case population [Brummel-Ziedins K E, Vossen C Y, Butenas S, Mann K G, Rosendaal F R. Thrombin generation profiles in deep venous thrombosis. J Thromb Haemost 2005; 3: 2497-505], comparisons of predicted thrombin generation profiles successfully stratified groups of individuals consistent with their documented incidences of thrombosis. A complementary analysis of the LETS control population, identified a subset of individuals whose thrombin generation profiles resembled those from the case population thus suggesting a potential application for risk prediction [Brummel-Ziedins K E, Vossen C Y, Butenas S, Mann K G, Rosendaal F R. Thrombin generation profiles in deep venous thrombosis. J Thromb Haemost 2005; 3: 2497-505; Brummel-Ziedins K, Vossen C Y, Rosendaal F R, Umezaki K, Mann K G. The plasma hemostatic proteome: thrombin generation in healthy individuals. J Thromb Haemost 2005; 3: 1472-81].

In general, both empirical and computational studies indicate that the course of thrombin formation appears to be an individual phenotypic property and thus methods that profile thrombin generation may have potential utility in the realm of clinical testing with respect to hemostatic balance and risk prediction [Brummel K E, Paradis S G, Butenas S, Mann K G. Thrombin functions during tissue factor-induced blood coagulation. Blood 2002; 100: 14852; Hemker H C. Begum S. Phenotyping the clotting system. Thromh Haemost 2000; 84: 747-51; Brummel K E, Paradis S G, Branda R E, Mann K G. Oral anticoagulation thresholds. Circulation 2001; 104: 2311-7; Brummel-Ziedins K, Whelihan M E, Ziedins E G, Mann K G. The resuscitative fluid you choose may potentiate bleeding. J Trauma 2006; 61: 1350-8; Brummel-Ziedins K, Vossen C Y, Rosendaal F R, Umezaki K, Mann K G. The plasma hemostatic proteome: thrombin generation in healthy individuals. J Thromb Haemost 2005; 3: 1472-81; Carr M E Jr, Martin E J, Carr S L. Delayed, reduced or inhibited thrombin production reduces platelet contractile force and results in weaker clot formation. Blood Coagul Fibrinolysis 2002; 13: 193-7; Hartert H S. The physical and biological constants of thrombelastography. Biorheology 1962; 1: 31-9; Hron G, Kollars M, Binder B R, Eichinger S, Kyrle P A. Identification of patients at low risk for recurrent venous thromboembolism by measuring thrombin generation. JAMA 2006; 296: 397-402]. These approaches provide an integrated assessment of the efficacy of an individual's coagulation proteome. An advantage in principle of the computational approach is that it allows one to rapidly evaluate the mechanistic contribution of specific factors and/or groups of factors to the overall thrombin profile of an individual or population.

When potential contributions to ACS and CAD by proteins essential to the blood coagulation process have been investigated empirically, no consistent changes in individual coagulation factor concentrations (apart from elevated fibrinogen) have been reported in ACS patients. However, it has been demonstrated that FIXa and FXIa are present in one out of four patients with acute myocardial infraction (MI) and sporadically in those with stable angina [Minnema M C, Peters R J, de Winter R, Lubbers Y P, Barzegar S, Bauer K A, Rosenberg R D, Hack C E, ten Cate H. Activation of clotting factors XI and IX in patients with acute myocardial infarction. Arterioscler Thromb Vase Biol 2000; 20: 2489-93]. Vaziri et al. [Vaziri N D, Kennedy S C, Kennedy D, Gonzales E. Coagulation, fibrinolytic, and inhibitory proteins in acute myocardial infarction and angina pectoris. Am J Med 1992; 93: 651-7] reported increased FIX and decreased FII and FV in ACS patients. Elevated FVIII, which predisposes to venous and arterial thrombosis [Martinelli I. von Willebrand factor and factor VIII as risk factors for arterial and venous thrombosis. Semin Hematol 2005; 42: 49-55] and reduced AT activity [Merlini P A, Bauer K A, Oltrona L, Ardissino D, Cattaneo M, Belli C, Mannucci P M, Rosenberg R D. Persistent activation of coagulation mechanism in unstable angina and myocardial infarction. Circulation 1994; 90: 61-8; Vaziri N D, Kennedy S C, Kennedy D, Gonzales E. Coagulation, fibrinolytic, and inhibitory proteins in acute myocardial infarction and angina pectoris. Am J Med 1992; 93: 651-7], can also be detected in ACS patients. No studies have reported the integrated contribution of the coagulation proteome in relation to thrombin generation as a discriminator between acute and stable CAD.

In this study, we investigate whether computational thrombin generation profiles, dependent upon the composition of the major coagulation proteome proteins, can discriminate between individuals with acute and stable CAD.

Materials and methods

### Materials

HEPES and ethylenediaminetetraacetic acid (EDTA) were purchased from Sigma Chemical Co. (St Louis, Mo., USA).

Human coagulation FVII, FX, FIX, and prothrombin, were isolated from fresh frozen plasma using the methods of Bajaj et al., and were purged of trace contaminants and traces of active enzymes. Human FV and AT were isolated from freshly frozen plasma [Katzmann J A, Nesheim M E, Hibbard L S, Mann K G. Isolation of functional human coagulation factor V by using a hybridoma antibody. Proc Natl Acad Sci USA 1981; 78: 162-6; Griffith M J, Noyes C M, Church F C. Reactive site peptide structural similarity between heparin cofactor 11 and antithrombin Ill. J Biol Chem 1985; 260: 2218-25]. Recombinant FVIII and recombinant TF (residues 1-242) were provided as gifts from Drs Shu Len Liu and Roger Lundblad (Hyland division, Baxter Healthcare Corp, Duarte, Calif., USA). Recombinant human FVIIa was provided as a gift from Dr Ula Hedner (Novo Nordisk, Denmark). Recombinant full-length TFPI was provided as a gift from Dr K. Johnson (Chiron Corp, Emeryville, Calif., USA).

The preparation of the TF/lipid reagent was performed as described elsewhere [Cawthem K M, van't Veer C, Lock J B, DiLorenzo M E, Branda R F, Mann K G. Blood coagulation in hemophilia A and hemophilia C. Blood 1998; 91: 4581-92]. 1,2-Dioleolyl-sn-glycero-3-phospho-L-serine (PS) and 1,2-dioleoyl-sn-glycero-3-phosphocholine (PC) were purchased from Avanti Polar Lipids, Inc (Alabaster, Ala., USA). Phospholipid vesicles (PCPS) composed of 25% PS and 75% PC were prepared as described [Higgins D L, Mann K G. The interaction of bovine factor V and factor V-derived peptides with phospholipid vesicles. J Biol Chem 1983; 258: 6503-8]. Spectrozyme TH was purchased from American Diagnostica, Inc (Greenwich, Conn., USA).

### Study Population

We enrolled 28 patients with ACS, aged between 46 and 76 years (six women), including 14 with STEMI, admitted to the Department of Hemodynamics with chest pain. These ACS patients had experienced chest pain up to 12 hours (h) prior to seeking treatment. Upon arrival at the hospital, informed consent was obtained and blood from patients was immediately drawn as part of the admitting procedure. We also enrolled 25 patients with angiographically confirmed stable CAD (>50% stenosis in at least one major coronary artery). Inclusion criteria for ACS patients were typical chest pain and either ST-elevation >0.1 mV in at least two contiguous leads, and elevated cardiac troponin levels. Exclusion criteria in ACS patients were as follows: cardiogenic shock, any acute illness, cancer, hepatic or renal dysfunction, history of venous thromboembolism or stroke, oral anticoagulant or heparin administration, previous coronary artery bypass grafting surgery. Myocardial infarction was defined according to the ESC/ACC criteria [Myocardial infarction redefined—a consensus document of The Joint European Society of Cardiology/American College of Cardiology Committee for the redefinition of myocardial infarction. Ear Heart J 2000; 21: 1502-13]. All patients took aspirin 300 mg 6 to 12 h before the study. None of the subjects received thienopyridimes prior to blood collection. Fifty percent of patients (n=14) had arterial hypertension, four had diabetes mellitus and 10 patients were current smokers. At the time of blood collection, patients with ACS had either been treated on a long-term basis with: aspirin 75 mg daily (n=14), β-blockers (n=13), angiotensin-converting enzyme (ACE) inhibitors (n=11), calcium antagonists (n=3) and statins (n=5).

Stable angina patients (Canadian Cardiovascular Society classes II or III), age between 46 and 76 years (n=25, seven women), were matched to the ACS patients for age and gender.

None of these patients developed ACS or underwent angioplasty within 6 months before the start of the study. Sixty percent of patients (n=14) had arterial hypertension and 20% had a history of myocardial infarction. Current smokers constituted 40% of stable patients (n=10). Patients reported taking aspirin 75 mg daily (n=24), (3-blockers (n=18), ACE inhibitors (n=12), calcium antagonists (n=3), nitrates (n=5) and statins (n=5). All patients with ACS and stable CAD had normal platelet counts (194 000-341 000 pL⁻¹). The University Ethical Committee approved the study, and patients provided written informed consent.

### Methods

Blood collection and coagulation protein analyses—Blood was drawn into 0.1 volume of 3.2% trisodium citrate from an antecubital vein with minimal stasis (within 15 min upon admission in case of ACS patients). Citrated blood samples were centrifuged (3000×g for 20 min) within 15 min of collection and stored in aliquots at −80° C. until further use. Lipid profiles, blood cell counts, glucose, creatinine, aminotransferases, cardiac troponin I (cTn1) and MB creatine kinase were assayed by routine laboratory techniques. There were no differences in glucose levels between both groups as well as in the percentage of patients with diabetes. Fibrinogen was determined using the Clauss method. Total TFPI was determined by ELISA (Diagnostica Stago, Asnieres, France). FIT, FV, FVII, FVIII, FIX, FX were measured by one-stage clotting assays using factor-deficient plasmas (Dade Behring, Liederbach, Germany). AT activity was measured using Berichrom (Dade Behring).

Computational model—The current numerical model is based upon prior publications by Jones et al. [Jones K C, Mann K G. A model for the tissue factor pathway to thrombin. 11. A mathematical simulation. J Biol Chem 1994; 269: 23367-73 [published erratum appears in J Biol Chem 1995 Apr. 14; 270(15):9026], Hockin et al. [Hockin M F, Jones K C, Everse S J, Mann K G. A model for the stoichiometric regulation of blood coagulation. J Biol Chem 2002; 277: 18322-33] and Butenas et al. [Butenas S, Orfeo T, Gissel M T, Brummel K E, Mann K G. The significance of circulating factor IXa in blood. J Biol Chem 2004; 279: 22875-82] and yields concentration vs. time profiles for selected species when electronic mixtures of the procoagulant FIT, FV, FVII, FVIIa, FVIII, FIX and FX, and the anticoagulants TFPI and AT are exposed to picomolar concentrations of TF.

These analytes were determined for each individual in the ACS (n=28) and stable CAD (n=25) populations. Each individual's blood factor concentration was entered into the computer database, and simulated reactions were initiated with 5 pm TF [Hockin M F, Jones K C, Everse S J, Mann K G. A model for the stoichiometric regulation of blood coagulation. J Biol Chem 2002; 277: 18322-33]. The standard control simulation sets zymogen, cofactor and inhibitor concentrations at their mean physiological values [Brummel-Ziedins K, Orfeo T, Jenny N S, Everse S J, Mann K G. Blood myocardial infarction associated with factor V Leiden or prothrombin 20210A. Circulation 1998; 97: 1037-41] and the concentration of the TF stimulus at 5 pm.

Simulated reactions were solved for active thrombin over 1200 s. The outputs of these active thrombin curves are evaluated by parameters that describe the initiation, propagation and termination phases of thrombin generation: maximum level of thrombin generated, maximum rate of thrombin generated, time to 2 nM thrombin (clot time), time to maximum level of thrombin generated and total thrombin generated (area under the curve) [Brummel-Ziedins K E, Vossen C Y, Butenas S, Mann K G, Rosendaal F R. Thrombin generation profiles in deep venous thrombosis. J Thromb Haemost 2005; 3: 2497-505].

Synthetic coagulation proteome model—The procedure used is a modification of Lawson et al. [Lawson J H, Kalafatis M. Stram S. Mann K H. A model for tissue factor pathway to thrombin. 1. An empirical study. J Biol Chem 1994; 269: 23357-66] and van't Veer et al. [van t Veer C, Mann K G. Regulation of tissue factor initiated thrombin generation by the stoichiometric inhibitors tissue factor pathway inhibitor, antithrombin-III, and heparin cofactor-11. J Biol Chem 1997; 272:4367-77]. I. Procofactor solution Relipidated TF (10 pM; molar ratio PCPS:TF=5000) is incubated with 4 μM PCPS in HBS (20 mM HEPES and 150 mm NaCl, pH 7.4), 2 nm CaCl₂ for 10 min at 37° C. Factor V (40 mM) and FVIII (1.4 nM) are added prior to the initiation of the reaction. II. Zymogen-inhibitor solution. Prothrombin (2.8 μM), FVII (20 nM), FVIIa (0.2 nM), FX (340 nM), FIX (180 nM), FXI (60 mM), TFPI (5 rim), and AT (6.8 μM) are preheated in HBS, 2 mm CaCl₂, at 37° C. for 3 min.

The reaction is started by mixing equal volumes of both Ca²⁺ pre-equilibrated solutions resulting in physiological concentrations of the zymogens, pro-cofactors and inhibitors, 5 pM TF, 2 mm CaCl₂ and 2 μM PCPS. After the start of the reaction, at selected time points, 10 μL aliquots are withdrawn from the reaction mixture and quenched in 20 mm EDTA in HBS (pH 7.4) containing 0.2 mm Spectrozyme TH and assayed immediately for thrombin activity. The hydrolysis of the substrate is monitored by the change in absorbance at 405 nm using a Vₘₐₓ spectrophotometer (Molecular Devices Corp., Menlo Park, Calif., USA). Thrombin generation is calculated from a standard curve prepared by serial dilutions of α-thrombin.

### Statistics

For comparison between groups, the t-test was used for normally distributed data and the Mann-Whitney U-test for non-normally distributed data. Differences between the groups are expressed as P-values. A P-value <0.05 was considered significant.

### Results

### ACS and CAD Populations

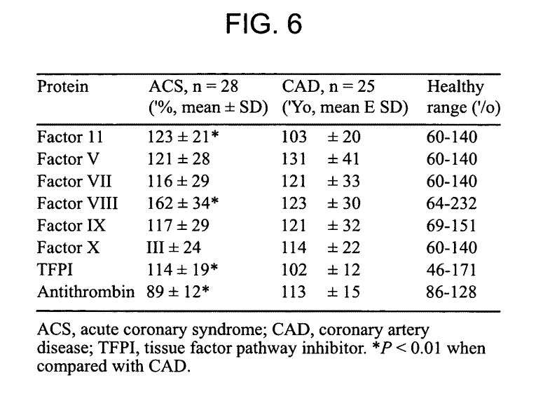

The mean blood coagulation factor levels of both the ACS and CAD populations were within the reference values for healthy individuals (See tabulation provided in FIG. 6). The concentrations of TFPI, AT, FII and FVIII differed significantly (P<0.01) between the groups, with levels of FIT, FVIII and TFPI higher and AT lower in ACS patients.

### Thrombin Generation

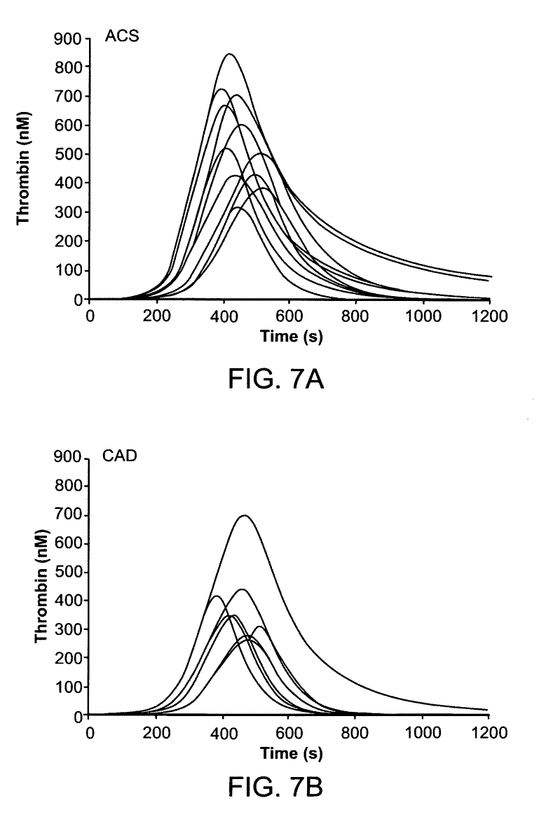

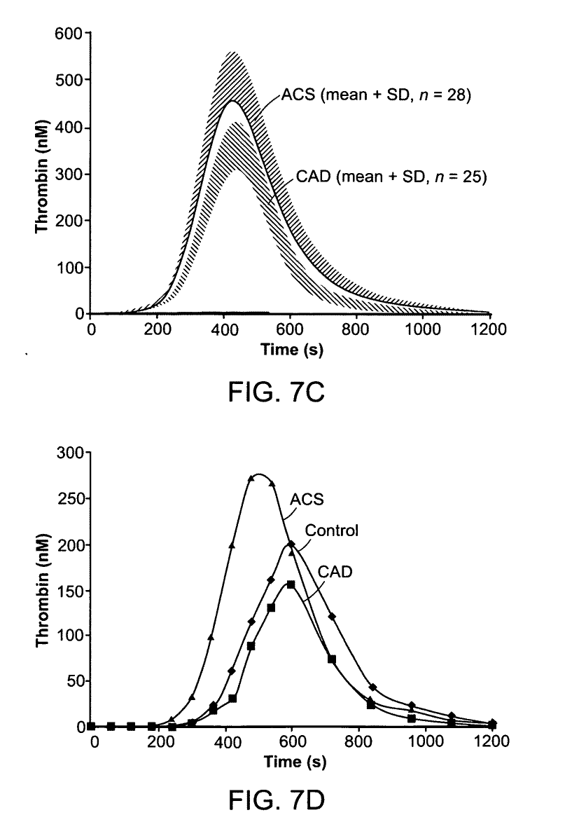

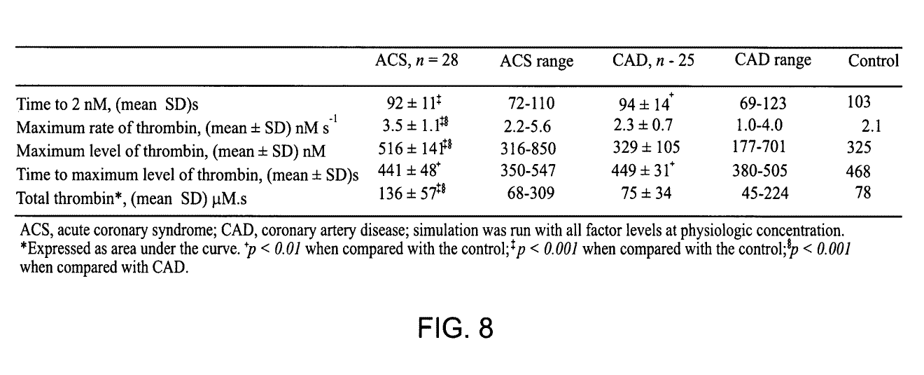

FIGS. 7A-D present the thrombin generation curves for each individual within the ACS (FIG. 7A) and the CAD (FIG. 7B) populations. An analysis was conducted in which all the thrombin generation curves within each population were averaged by combining thrombin second by second over the time course of 1200 s. The resulting mean thrombin concentration over time simulation and the positive standard deviation is presented for each group in FIG. 7C. The simulated thrombin parameters are presented in the tabulation provided in FIG. 8 as the mean±SD and range for the ACS and CAD populations and compared with a control reflecting mean physiologic concentrations. Significantly higher maximum levels, rates and total thrombin are seen when the ACS population is compared with the CAD population (P<0.001).

The mean factor concentrations of the ACS and CAD populations (See tabulation in FIG. 6) were used in empirical coagulation proteome experiments with a 5 pM TF stimulus. These results are shown in FIG. 7D. By recapitulating the mean procoagulant and anticoagulant protein profiles of the ACS and CAD populations in the presence of a phospholipid surface and TF, we obtain thrombin generation profiles for the populations that mimic the pattern observed in the numerical simulation in FIG. 7C.

### Normalization of Thrombin Generation

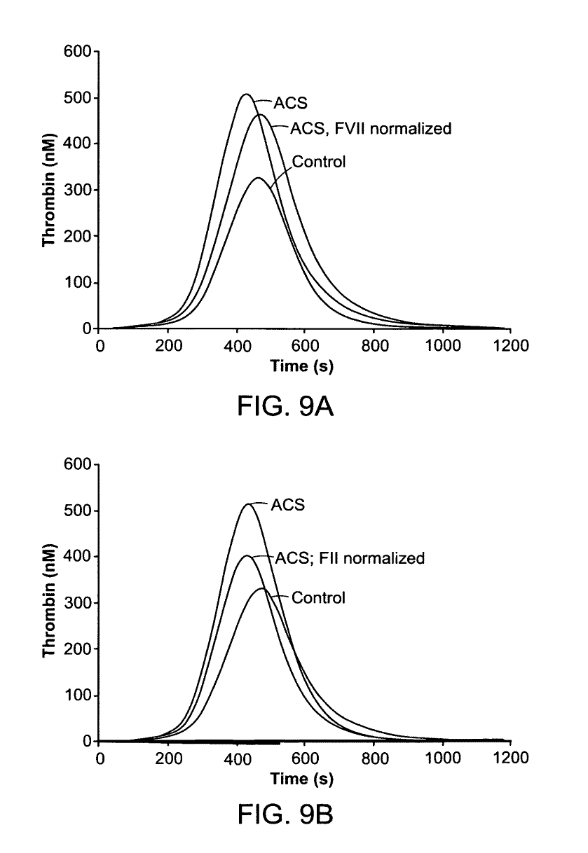

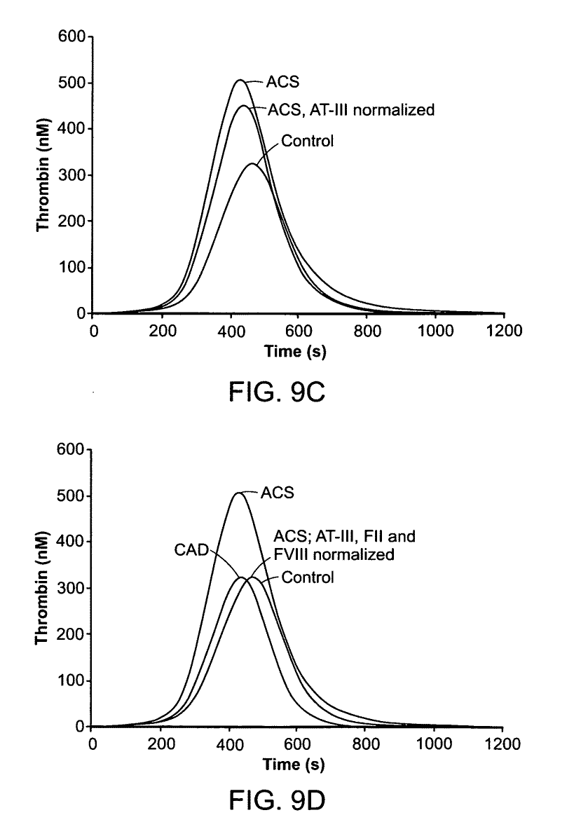

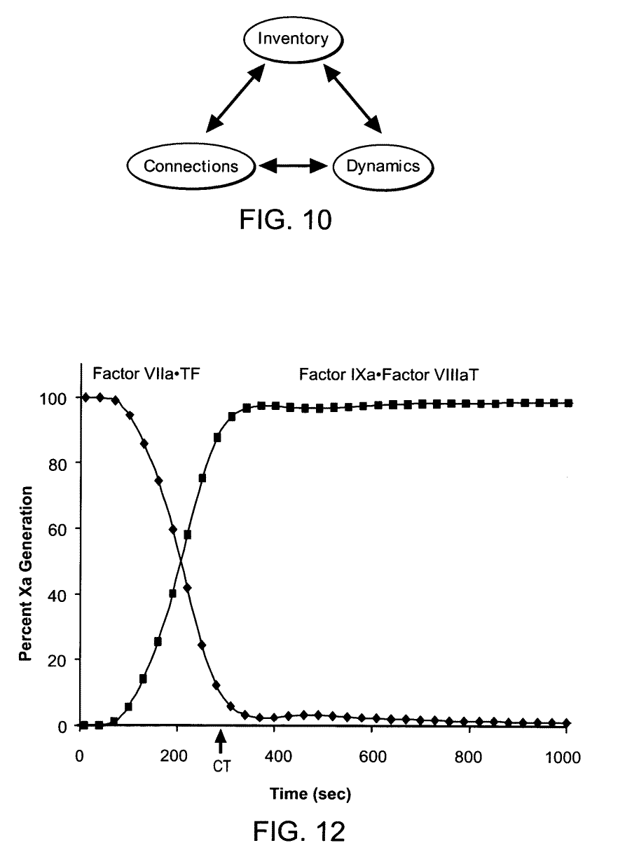

We evaluated the contribution of each individual protein to the overall simulated thrombin generation profile of the ACS population. The goal was to identify those protein(s) concentrations which might account for the difference in thrombin generation profiles between the ACS and CAD populations vs. the healthy control. Thrombin generation profiles were initially computed by successively holding each protein (eight total) at their mean physiologic value while the other seven were used at their mean ACS population value. We evaluated the outcomes by comparing the magnitude of the shift in the thrombin generation profiles induced by normalizing each factor individually. These analyses indicated that normalized FVIII, AT and prothrombin individually caused the largest changes to the thrombin generation profiles; however, none by themselves were sufficient to restore thrombin to a control or CAD level (FIG. 9A-C). Continuing this procedure by normalizing groups of factors simultaneously, we determined that the combination of FVIII, AT and prothrombin produced a thrombin profile that was congruent to that for the control and CAD (FIG. 9D).

### Discussion

Our data indicate that hypothetical thrombin generation based upon coagulation factor composition can distinguish between acute and stable CAD. Individuals within the ACS population generated significantly higher levels of thrombin at a 50% faster rate resulting in more thrombin over the time course of the TF-initiated coagulation reaction. The prothrombotic thrombin generation profiles of individuals with ACS appear to be driven primarily by collective alterations in FVIII, AT and FII levels. These results suggest that a specific array of coagulation factor and inhibitor levels, all largely within the normal range, can potentially contribute to the procoagulant phenotype in acute phases of CAD. This supports the concept that ‘vulnerable’ circulating blood with prothrombotic alterations and a vulnerable plaque, a potential source of TF, may both play a role in the development of ACS.

Overall, this study suggests that the integration of blood composition data into an assessment of thrombin generation potential can discriminate between acute and stable CAD and that a limited array of factors can be predictive.

### Example 2

### Introduction

Therapeutic agents that regulate blood coagulation are critical to the management of thrombotic disorders and surgical interventions. Many new compounds that were plausible in terms of target, relative specificity, mechanism and kinetic and thermodynamic properties have failed to perform as expected when assessed, at significant expense, in vivo. The mathematical model and methodology of the present invention describes the dynamic biochemical repair process that emerges in response to vascular injury and allows us to incorporate hypothetical inhibitors/enhancers at their proposed sites of interaction, thus providing an assessment of their efficacy. In conjunction with our coagulation proteome model (a synthetic reconstruction using purified protein components represented in the mathematical model), we evaluated a set of anticoagulants (unfractionated heparin, synthetic heparin pentasaccharide, and inhibitors of factor Xa and thrombin) in experimental regimens (as described herein) relevant to prophylactic and therapeutic usages. The interactions of each anticoagulant with its target(s) were incorporated into the overall mathematical description of the reaction pathways. Outcomes for each anticoagulant at clinically relevant concentrations were generated and compared with results from empirical reconstructions.

### Experimental Procedures

Materials—Human coagulation fVII, fX, fIX, and prothrombin (fII), were isolated from fresh frozen plasma using the methods of Bajaj et al., and were purged of traces of active enzymes. FXI was purified using the monoclonal anti-fXI antibody α-fXI-2. Human fV and AT were isolated from freshly frozen plasma. The following were provided as gifts: recombinant fVIII and recombinant Tf (residues 1-242); recombinant human fVIIa; and recombinant full-length tissue factor pathway inhibitor (TFPI). Corn trypsin inhibitor (CTI) was isolated from popcorn. The preparation of the Tf/lipid reagent was performed. 1,2-Dioleolyl-sn-Glycero-3-Phospho-L-Serine (PS) and 1,2-Dioleoyl-sn-Glycero-3-Phosphocholine (PC) were purchased from Avanti Polar Lipids, Inc (Alabaster, Ala.), and EDTA was purchased from Sigma (St Louis, Mo.). Phospholipid vesicles (PCPS) composed of 75% PC and 25% PS and were prepared.

Spectrozyme TH was purchased from American Diagnostica, Inc (Greenwich, Conn.) and D-Phe-Pro-ArgCH₂Cl (FPRck) was prepared. UFH (145 IU/mg) was purchased from Sigma, dansylarginine-N-[3-ethyl-1,5-pentanediyl]amide (DAPA) was purchased from Haematologic Technologies Inc. (Essex Junction, Vt.) and fondaparinux (Fpx) (GlaxoSmithKline, Research Triangle Park, N.C.) was purchased from Fletcher Allen Health Care (Burlington, Vt.). Benzenesulfonyl-D-Arg-Gly-Arg-ketothiazole (C921-78) was provided as a gift. ELISA thrombin-AT (TAT) kit (Enzygnost TAT) was purchased from Behring (Marburg, Germany).

Computational Model The current mathematical model as described herein was used, which yields concentration versus time profiles for selected species when electronic mixtures of the procoagulants fII, fIX, fX, fVII, fVIIa, fV, and fVIII and the anticoagulants TFPI and AT are exposed to picomolar concentrations of Tf. An additional step of AT inhibition of fXa when associated with fVa in the prothrombinase complex was incorporated using a kinetic constant reflecting in house and literature values. Numerical simulations of resupply were constructed to mimic the equal volume addition of fresh “plasma” material used in the empirical models.

Modeling each anticoagulant presents a number of issues. With UFH, these included the number of targets, the mechanistic complexities, both of the interaction of heparin species with AT and of the interactions of the AT heparin complex with each susceptible protease, and the diversity in source and fractionation state of the UFH used to establish the kinetic data. Simplifying assumptions were made which included: the reduction of mechanisms involving assembly steps followed by conformational transitions to one step collision-based transitions controlled by global constants of inhibition; the averaging of published kinetic data derived from experiments using different UFH preparations and different UFH/AT molar ratios; and the use of data collected at physiologic ionic strength and, when important, physiologic Ca²⁺ concentration. Similar approaches were applied to Fpx, although the uniformity of the preparations is not an issue.

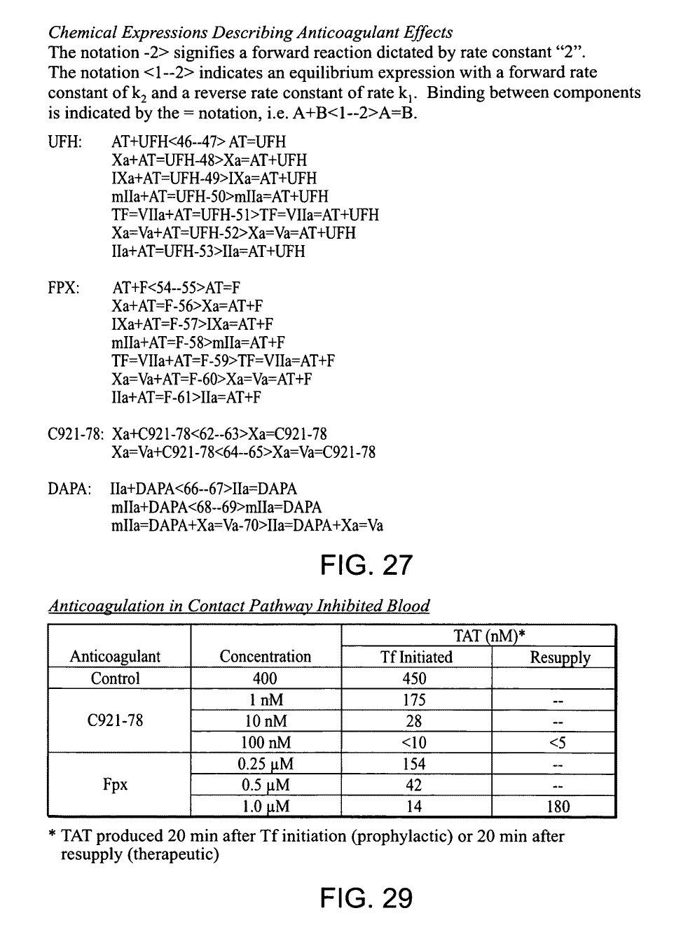

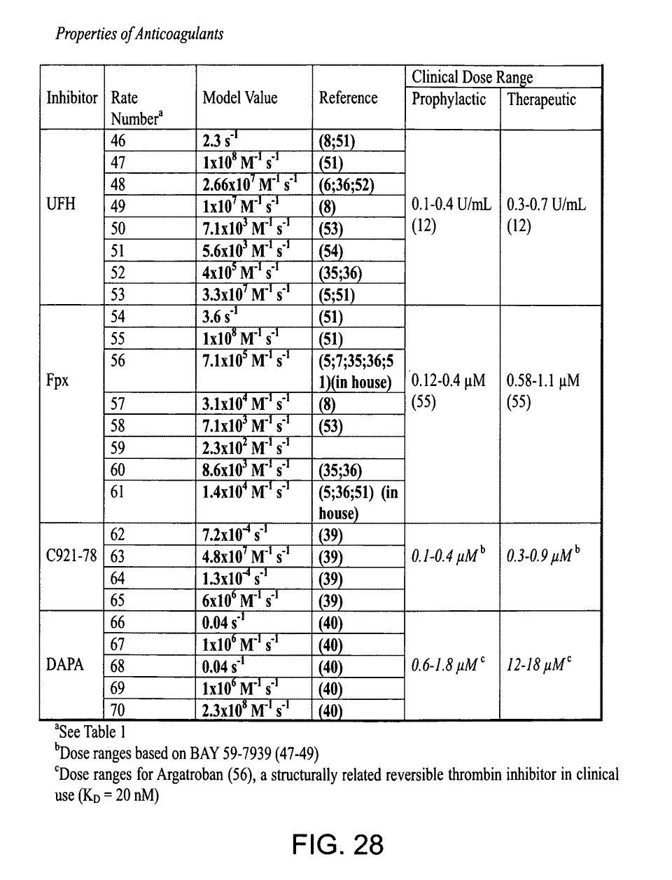

In addition, 2 groups have reported that in the presence of fII, fXa in prothrombinase is protected from inhibition by AT bound to UFH or Fpx and that the magnitude of this protection exceeds that predicted by a straightforward competition for binding to fXa. The magnitude of the prothrombin protective effect, established by different experimental techniques, varied from 50 to 100 fold to 1000 fold. A mechanism applicable to this effect has been advanced but remains controversial. Complicating modeling efforts further, Tf-initiated reactions in closed model systems often display complete prothrombin consumption during the progress of the reaction. Because of the disparity in the magnitude of the reported rate constants and the lack of a consensus mechanism explaining the effect, several models were constructed on the basis of rate constants for AT heparin inhibitory effects on prothrombinase derived in the absence of prothrombin and with differing rate constants reported for the protective effect. The outputs of these various models were compared to data on the UFH effects on the progress of Tf-initiated reactions in the synthetic coagulation proteome (not shown). Based on these comparisons, a mechanism (see tabulation provided as FIG. 27) that incorporated the role of prothrombin in protecting prothrombinase from inhibition by AT=UFH and AT=Fpx and a set of rate constants (see tabulation provided as FIG. 28, rate numbers 52 and 60) were selected.

C921-78 has been reported to specifically and reversibly inhibit fXa and fXa when associated with fVa on membranes (prothrombinase). The authors initially proposed a one step reversible association model for the inhibition of fXa: the equilibrium was characterized by kₒₙ values between 10⁷ to 10⁸M⁻¹ s⁻¹ and k_{off} values from 10⁻⁴ to 10⁻⁵ s⁻¹, yielding a K_{D} for fXa of ˜14 pM and 22 pM for fXa in prothrombinase. Further experimental analysis led the authors to suggest a more complex multistep mechanism involving a relatively low affinity binding interaction followed by an isomerization event that yielded a highly stabilized complex with fXa. Modeling of these 2 mechanisms yielded different thrombin generation profiles at a given C921-78 concentration, with the more complex pathway clearly underestimating the efficacy of C921-78 in the synthetic coagulation proteome (data not shown). Therefore the one step reversible association model was used.

DAPA is a reversible, active site directed inhibitor of thrombin and meizotirombin (mIIa), with a K_{D} of 20-40 nM. The mIIa=DAPA intermediate is processed to IIa=DAPA by prothrombinase. Similar to Argatroban, a structurally related thrombin inhibitor in clinical use, the k_{off} for the DAPA thrombin complex is in the range of 0.04 to 0.07 s⁻¹.

Pharmacologic agents were modeled by adding the appropriate sets of equations (FIG. 27) describing their activities to the existing framework of differential equations. It should be recognized that these equations are usable in the methods of the present invention similar to that described herein for the equations of FIG. 5. The rate constants employed represent average values reflecting literature and in house measurements (FIG. 28). Thrombin concentration at each time point is given as the sum of α-thrombin and meizothrombin concentrations, reflecting the fact that both species cleave the chromogenic assay employed in the empirical assays. Activity based concentrations for UFH were converted to molar values for modeling using a specific activity of 1 IU/mL=170 nM functional heparin molecules.

Synthetic Coagulation Model—The procedure used is a modification of Lawson et al. [Lawson, J. H., Kalafatis, M., Stram, S., and Mann, K. G. (1994) J. Biol. Chem. 269, 23357-23366] and van't Veer et al. [van't Veer, C. and Mann, K. G. (1997) J. Biol. Chem. 272, 4367-4377]. Relipidated Tf at 5 pM final concentration was added to a mixture of fII, fV, fVII, fVIIIa, fVIII, fIX, fX, fXI, TFPI, and AT (all at mean physiologic concentrations) (43) in 20 mM HEPES/150 mM NaCl (HBS) with 2 mM CaCl₂ containing 2 μM PCPS. Pharmacologic agents were incorporated into the reaction mixture prior to addition of the Tf reagent. Resupply was conducted at 20 min post Tf initiation by the addition of an equal volume of a freshly constituted, Tf free mixture containing procofactors, zymogens and inhibitors in HBS with 2 mM CaCl₂ and 2 μM PCPS to an ongoing reaction. Pharmacologic agents were present in the resupply mixture at twice their final concentration.

Thrombin generation over time was measured in a chromogenic assay using Spectrozyme TH and a THERMOₘₐₓ microplate reader (Molecular Devices Corp., Menlo Park, Calif.). In experiments assessing the reversible thrombin inhibitor DAPA, rates of thrombin generation were calculated by reference to a standard curve relating thrombin concentration to rates of substrate hydrolysis in the presence of relevant DAPA concentrations.

Whole Blood Model—The protocol used is a modification of Rand et al. [Orfeo, T., Butenas, S., Brummel-Ziedins, K. E., and Mann, K. G. (2005) J. Biol. Chem. 280, 42887-42896; Rand, M. D., Lock, J. B., van't Veer, C., Gaffney, D. P., and Mann, K. G. (1996) Blood 88, 3432-3445]. A healthy donor was recruited, advised according to a protocol approved by the University of Vermont Human Studies Committee and their consent was obtained. The individual selected exhibited normal values for the parameters of blood coagulation, protein levels and platelet counts. Blood was drawn by venipuncture and immediately delivered into the reagent-loaded tubes. In general, 2 sets of aliquots of CTI treated blood were initiated with 5 uM Tf reagent. One set was quenched at 1 min intervals while the other was incubated for 20 min. At 20 min after initiation with Tf, the unquenched series of tubes, which all showed visible clots at ˜4 min, received an equal volume of new blood, drawn two minutes prior to its use from the same individual. This resupply blood contained CTI alone (0.1 mg/ml), CTI+2 uM fondaparinux or CTI+320 nM C921-78. After the experiment, the tubes are centrifuged and the supernatants are aliquoted and analyzed for TAT levels. A subset of tubes containing either Fpx or C921-78 were initiated with 5 pM Tf reagent, quenched at 20 min and analyzed for TAT levels. In the absence of exogenous Tf, the CTI blood from the first draw clotted at 37 min, while CTI blood from the second draw that was not used in the resupply experiment clotted after 35 min, demonstrating the absence of contact pathway contributions during the experimental time frame.

Gel Electrophoresis and Western Blotting—Resupply time course samples were quenched into an equal volume of 50 mM EDTA, 50 μM FPRck in HBS and 4 μl aliquots (potentially containing 100 ng prothrombin) were made 2% in SDS and 2% in β-mercaptoethanol and analyzed by SDS-PAGE. Protein species were separated on 4 to 12% linear gradient gel and transferred to nitrocellulose membranes (Bio-Rad Laboratories Inc., US). FII and its activation products were probed for using a polyclonal burro anti-human prethrombin 1 antibody. Reactive bands were detected using goat anti-equine horseradish peroxidase conjugated IgG (Southern Biotechnology, Birmingham Ala.) with a chemiluminescence reagent (Perkin Elmer, Boston Mass.) and Kodak X-OMat film.

### Results

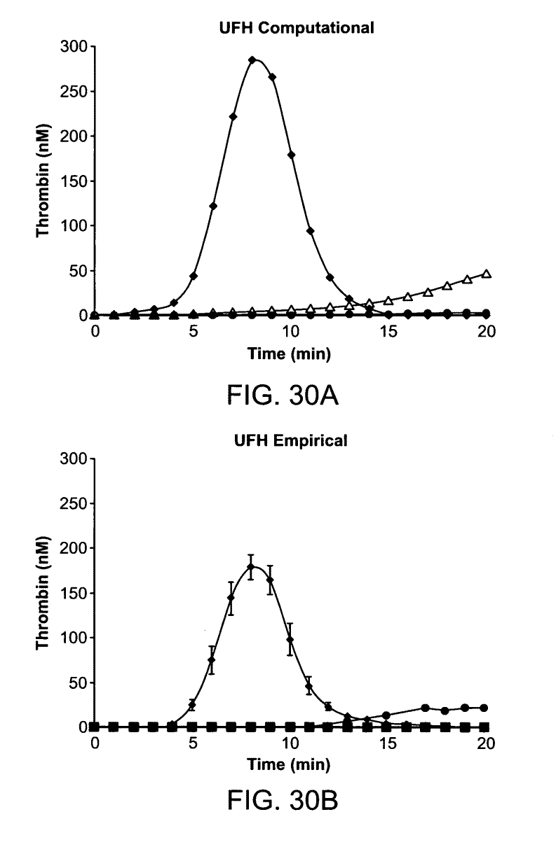

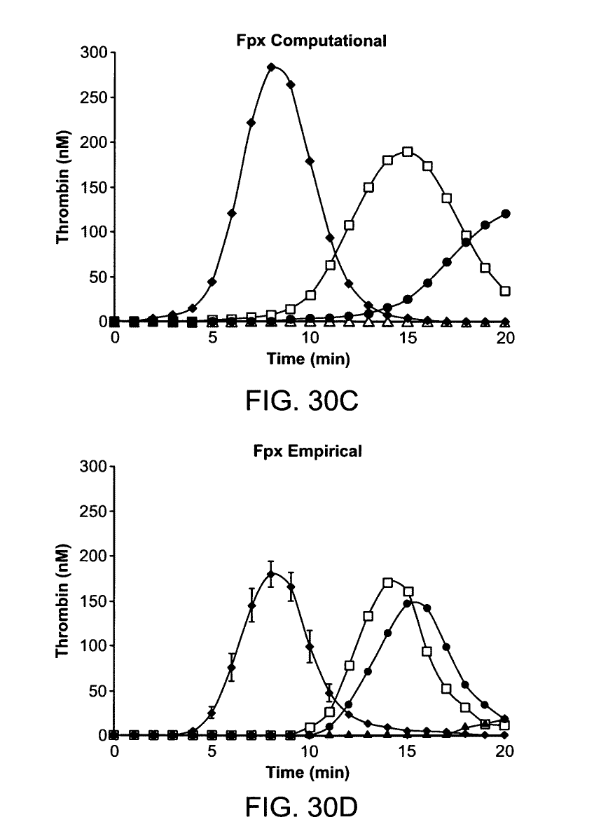

Incorporation of Anticoagulants into the Mathematical Model—FIG. 27 presents the sets of equations describing the mechanisms of action of each of the anticoagulants used in this study and FIG. 28 presents the rate constants controlling the identified bimolecular interactions and the dosing ranges recommended for prophylactic and therapeutic applications. Details of modeling the various anticoagulant interactions are described herein and in the Methods section.
Prophylactic Anticoagulation—There is shown in FIGS. 30A and 30 C computational assessments and in FIGS. 30B and D empirical assessments of the efficacy of each anticoagulant in suppressing the onset of Tf-initiated thrombin formation. In both empirical and computational studies, a 5 pM Tf reagent stimulus was used and thrombin generation assessed over 20 min. Results are presented that compare thrombin generation in the absence of anticoagulant (♦) to that observed over the range of anticoagulant concentrations that drive the transition from partial to maximum suppression of thrombin formation. The enhanced suppression of thrombin levels in the presence of UFH and DAPA has 2 mechanistic routes, distinguishable on the basis of prothrombin consumption: neutralizing free thrombin in the system as it is produced by prothrombinase (i.e., prothrombin is consumed) and blocking the Tf-initiated process from the onset by inhibiting the initial thrombin dependent activation of fV and fVIII effectively preventing prothrombin consumption. Computational data are presented in terms of functional thrombin concentrations unless the time courses of thrombin production and prothrombin consumption diverge. To facilitate comparisons between modeling and empirical studies, the same symbol is used for a given concentration of an anticoagulant.

Indirect (AT Dependent) Inhibitors

UFH: The computational assessment of UFH (1 U/mL=170 μM functional heparin) indicated that suppression of the Tf-initiated process would be achieved at ˜0.07 U/mL, representing a 0.35% occupancy of the AT pool at the onset of the reaction. At 0.05 U/mL (FIG. 30A, ), the onset of thrombin formation was delayed from 3 (FIG. 30A, ♦) to 14 min, with active thrombin levels at 20 min only approaching 2 nM. When assessed in the synthetic coagulation proteome, 0.05 U UFH/mL (FIG. 30B, ) suppressed the onset of thrombin formation from 4 to 11 min with thrombin levels at 20 min reaching ˜20 nM. At 0.07 U/mL (FIG. 30B, ▪) thrombin generation was effectively suppressed. Thus, predicted and observed sensitivities to UFH were similar, with effective “prophylaxis” at 0.07 U/mL, a level near a prophylaxis dose (0.1 U/mL) in the clinical setting (FIG. 28).

Fpx—The computational assessment of Fpx efficacy predicted that Fpx concentrations in the range of 0.5 M, representing ˜15% occupancy of the AT pool at the onset of the reaction, would be required to suppress thrombin generation over 20 min (FIG. 30C, Δ). Lower levels of Fpx in the reaction, 125 nM (FIG. 30C, □) and 250 nM (FIG. 30C,: ) delayed the onset, maximum rate and maximum levels of the thrombin produced when compared to the control (FIG. 30C, ♦). Evaluation of the efficacy of Fpx in the synthetic coagulation proteome showed a similar concentration dependence, although the computationally predicted suppression was slightly more robust.

At 1 μM Fpx (FIG. 30D, ▴), the onset of thrombin generation was delayed to 17 min, with the concentration at 20 min approaching 20 nM, a level achieved in the absence of anticoagulant (FIG. 30D, ♦) within ˜5 min of Tf initiation. Thus, predicted and observed sensitivities to Fpx differed by ˜2 fold, with the computational prediction for effective “prophylaxis” being in line with the recommended clinical usage for prophylaxis (FIG. 28).

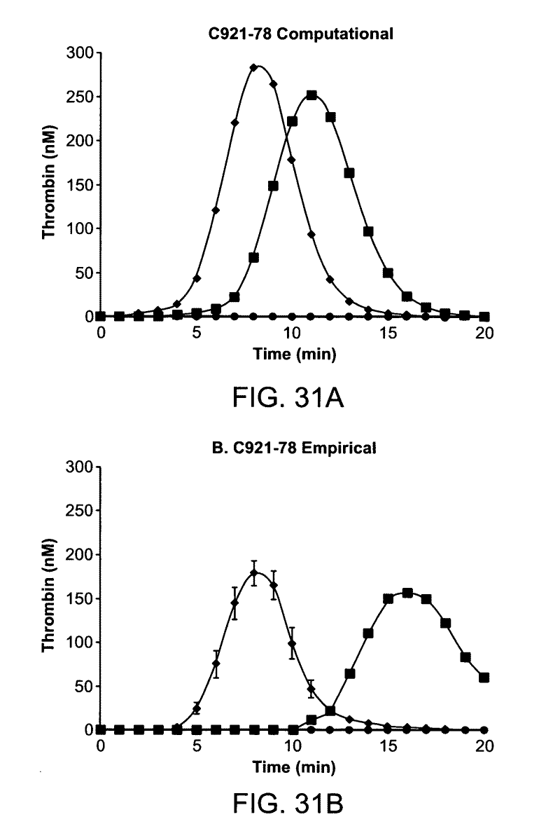

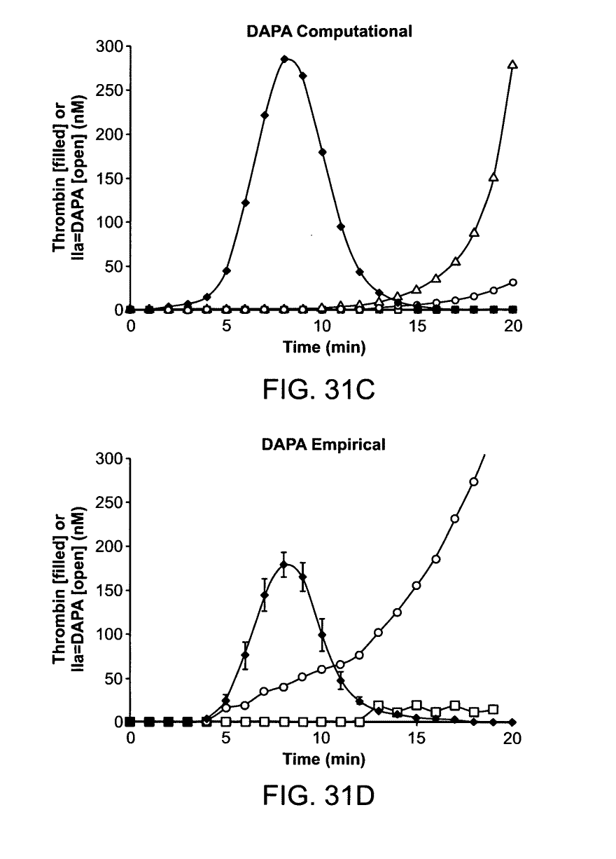

Direct (AT Independent) Inhibitors FXa inhibitor (C921-78)—The computational assessment of C921-78 efficacy predicted that a concentration of 10 nM, ˜500 times the K_{D} for its modeled targets (FIG. 27), would be required to totally suppress thrombin generation over 20 min (FIG. 31A, ). At a concentration ˜50 times greater than its K_{D} for its targets (FIG. 31A,: ▪), the onset time of thrombin generation was predicted to double, with maximum rates and levels of thrombin produced achieving almost control values. When C921-78 was tested in the synthetic coagulation proteome, 10 nM C921-78 completely suppressed thrombin generation (FIG. 31B, ), in line with modeling predictions. At 1 nM in the synthetic coagulation proteome (FIG. 31B, ▪), C921-78 proved somewhat more effective than computationally predicted in displacing the onset of thrombin generation, but did show the predicted minimal effects on maximum rate and concentrations of IIa. Direct comparison of these results to clinical usage recommendations for C921-78 is not possible; with another direct fXa inhibitor, an effective prophylaxis range between 450 and 1800 times the K_{D} for fXa has been reported.

IIa inhibitor (DAPA)—There sis shown in FIGS. 31C, D a computational (FIG. 31 C) and an empirical (FIG. 31D) assessments of the reversible thrombin inhibitor DAPA. Computational assessments indicated that suppressing the onset of prothrombin consumption would require levels of DAPA greater than 5 μM. At these DAPA concentrations, no less than 99.2% of any thrombin formed would be present as IIa=DAPA complex (FIG. 27, K_{D}=40 nM). Thus DAPA efficacy is computationally assessed by following the IIa=DAPA species. Similarly preliminary experiments in the synthetic coagulation proteome indicated that the dilution scheme used in the thrombin activity assay, and the presence of a chromogenic substrate at a concentration ˜80×Kₘ, “permitted” the appearance of active thrombin by shifting it from its association with DAPA. This buffering effect of reversible thrombin inhibitors has been described previously. Thus assayable thrombin is assumed to be present as IIa=DAPA and empirical results are also presented in terms of the formation of IIa=DAPA complex.

The computational assessment of DAPA efficacy predicted that levels of DAPA approaching 100 μM (5000×K_{D}, FIG. 28) would be required to suppress Tf-initiated thrombin generation (FIG. 31C, □). At 10 μM DAPA (FIG. 31C, Δ) the onset of IIa=DAPA formation was delayed to ˜10 min but levels of IIa=DAPA approaching the levels of active thrombin reached in the absence of DAPA (FIG. 31C, ♦) were achieved by 20 min, indicating similar levels of prothrombin consumption. Twenty μM DAPA (FIG. 31C, ◯) further prolonged the onset of prothrombin consumption, yielding ˜25 nM IIa=DAPA at 20 min. In the synthetic coagulation proteome DAPA proved less effective than predicted by the modeling, with 20 μM (FIG. 31D, ◯) only attenuating the maximum rate of prothrombin consumption. At 100 μM (FIG. 31D, □) suppression was more effective, delaying the onset and reducing total prothrombin consumption to ˜20 nM.

Both computational and empirical assessments indicate that the effectiveness of DAPA, or any related reversible thrombin inhibitor, as an anticoagulant for prophylaxis depends primarily on the sequestration of thrombin after it is produced rather than suppressing its production. Complete blockade of the Tf-initiated processes leading to robust prothrombin activation is not accomplished when concentrations relevant to clinical use are involved. For example, prophylactic use of Argatroban, a structurally related reversible IIa inhibitor, involves achieving plasma concentrations 30 to 90 times its K_{D} (0.6 to 1.8 μM) for thrombin (FIG. 28).

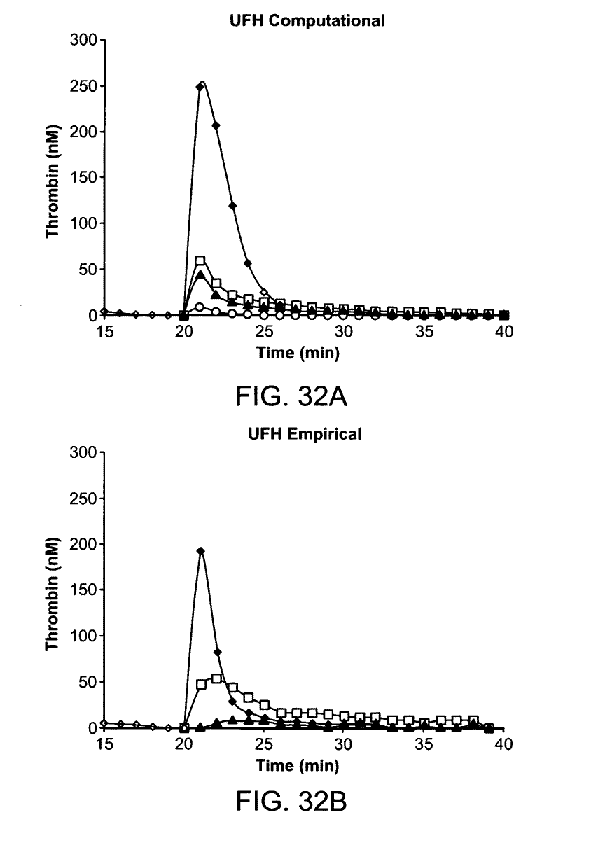

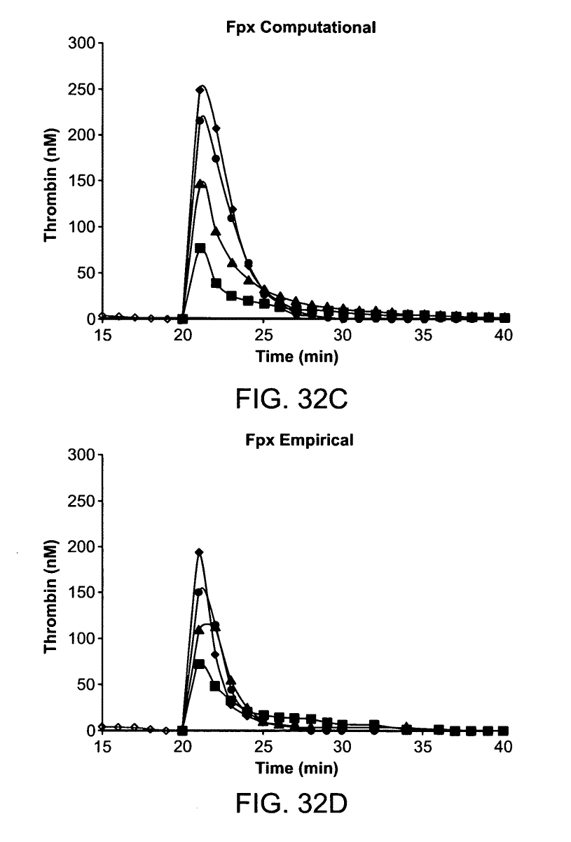

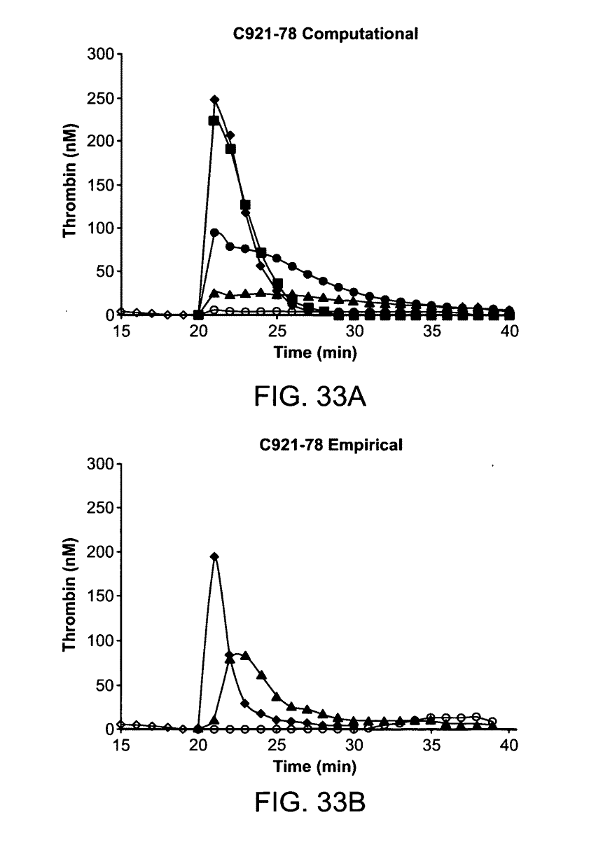

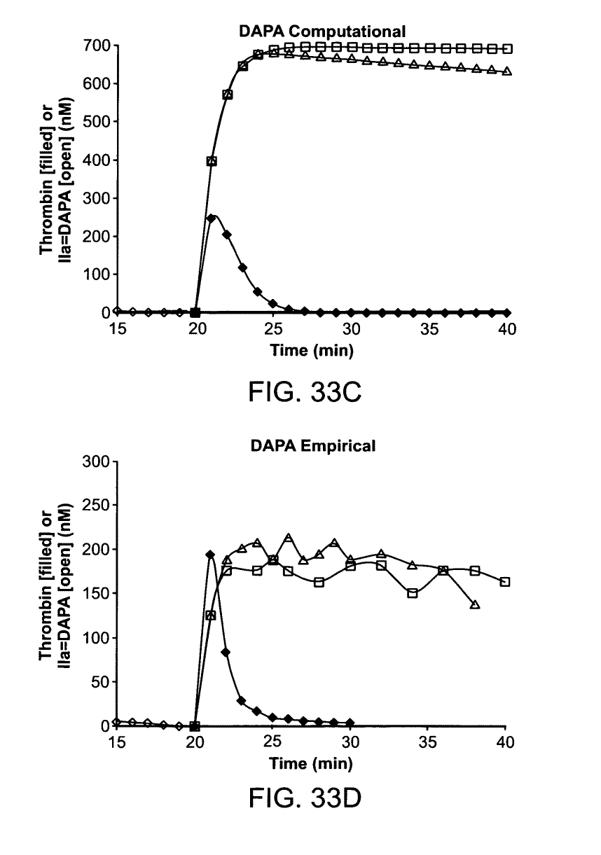

Therapeutic Anticoagulation—We have previously reported that one outcome in closed model systems of Tf-initiated coagulation is the formation of a relatively stable prothrombin activating potential which responds to the introduction of a new supply of reactants with an immediate burst of thrombin production, i.e., a “resupply response” (see U.S. application Ser. No. 10/507,661). There is shown in FIGS. 32A-D and 33A-D computational (FIGS. 32A,C; FIGS. 33A,C) and representative empirical (FIGS. 32B,D; FIGS. 33B,D) assessments of the efficacy of each anticoagulant in suppressing the resupply response. Computational and empirical time courses of thrombin formation are shown for the final 5 min of the Tf-initiated process and for the resupply phase.

### Indirect (AT Dependent) Inhibitors

UFH—The computational assessment of UFH efficacy indicated that complete suppression of the resupply response was not achievable. At 5.9 U/mL (FIG. 32A, ◯) thrombin levels reached 22 nM within 5 s and were ˜10 nM after 60 s. Even at a UFH concentration sufficient to bind 100% of the available AT (˜15.8 U/mL) thrombin levels were predicted to reach ˜12 nM within 5 s and be at ˜7 nM after 60 s. Thus, the computational assessment predicts that reaching the level of suppression achieved in the Tf-initiated process (e.g., FIG. 30A, ) requires ˜200 fold higher concentrations of UFH. However, when assessed in the synthetic coagulation proteome, UFH proved more effective than computationally predicted. At 0.5 U/mL (FIG. 32B, ▴) assayable thrombin was almost completely suppressed. Relative to UFH suppression of the Tf-initiated process (FIG. 30B, ▪), suppression of the resupply response required ˜5 fold higher UFH, with this concentration being within the therapeutic dosing range (Table 2). Interestingly, the level of suppression observed at 0.25 U/mL (FIG. 32B, □) was quite similar to that predicted for that concentration of UFH (FIG. 32A, □). This suggests that the discrepancy between the predicted and empirical sensitivities to UFH might reflect nonspecific effects (non AT dependent) in the proteome mixture by material in the UFH preparation. Approximately 60 to 70% of the mass in UFH preparations is not functional with respect to inducing the desired conformational change in AT.

Fpx—The computational assessment of Fpx efficacy indicated that there was no Fpx concentration that would lead to complete suppression of the resupply response. Increasing suppression was predicted as the percentage of the AT pool complexed with Fpx was raised (e.g., from 9% (FIG. 32C, ) to 37% (FIG. 32C, ▴). However at a concentration representing 100% saturation of the available AT pool (FIG. 32C, :▪), thrombin levels still approached 75 nM at 1 min, ˜30% of the level achieved in the absence of anticoagulant (FIG. 32C, ♦). Fpx efficacy in the synthetic coagulation proteome did not differ greatly from the computational predictions, showing a similar sensitivity to the concentration of Fpx, with a similar limit to the overall susceptibility of the resupply response to Fpx-mediated inhibition.

At 5 μM Fpx (FIG. 32D, ▪), which represents a 1.8 fold molar excess of Fpx over initially available AT in the resupply reaction, thrombin levels at 1 min reached ˜70 nM, or 35% of the maximum level observed in the absence of anticoagulants (FIG. 32D, ♦). The inability of Fpx to fully suppress thrombin generation during resupply in either mathematical or empirical models contrasts with its predicted and empirically observed suppression of the Tf-initiated process (FIGS. 30 and 30D). This data suggest that Fpx anticoagulation is more suited to prophylactic than therapeutic usage.

### Direct (AT Independent) Inhibitors

Direct fXa inhibitor (C921-78)—The computational assessment of C921-78 efficacy indicated that effective suppression of the resupply response could be achieved. FIG. 33A shows thrombin generation time course after resupply in the presence of 1 nM (▪), 10 nM (), 40 mM (▴) and 160 nM (◯) C921-78. Reduced but significant thrombin generation is indicated at 10 nM, the concentration that is effective in suppressing the Tf-initiated process. At 160 nM C921-78, thrombin generation is predicted to remain below 5 nM over 20 min.

C921-78 efficacy in the synthetic coagulation proteome exhibited similar concentration dependence. FIG. 33B shows representative thrombin generation time courses after resupply in the presence of 40 nM (▴) and 160 nM (◯). At 70 nM C921-78, maximum thrombin levels of 35 nM were observed at 4 min post resupply while at 100 nM maximum thrombin levels of ˜25 nM were observed at 10 min post resupply (not shown). At 160 nM C921-78 thrombin generation was delayed 11 min with maximum thrombin levels of 14 nM (FIG. 33B, ◯). Although the computational assessment predicted a slightly higher level of efficacy for C921-78, empirical and computational assessments both indicate that this anticoagulant regimen can achieve effective suppression of the resupply response, but that this will require concentrations 15 to 20 times higher than those needed to block the Tf-initiated process.

Direct IIa inhibitor (DAPA)—FIG. 33C shows the computational assessment of the resupply response in the presence of 10 μM (Δ) and 100 μM (□) DAPA. Identical rates of IIa=DAPA formation and identical final levels (700 nM) of IIa=DAPA are predicted for the 2 concentrations, suggesting that DAPA is not influencing the catalytic events leading to thrombin formation. This is in contrast to the ability of DAPA to partially attenuate Tf-initiated thrombin formation. FIG. 33D shows the corresponding empirical study of the effect of 10 μM (Δ) and 100 μM (□) DAPA on the resupply response. No substantial difference in rate or maximum final level is observed between the 2 DAPA concentrations. However, computational and empirical assessments differ in the final level of IIa=DAPA present, suggesting the possibility that prothrombin consumption in the synthetic coagulation proteome is incomplete in the presence of DAPA.

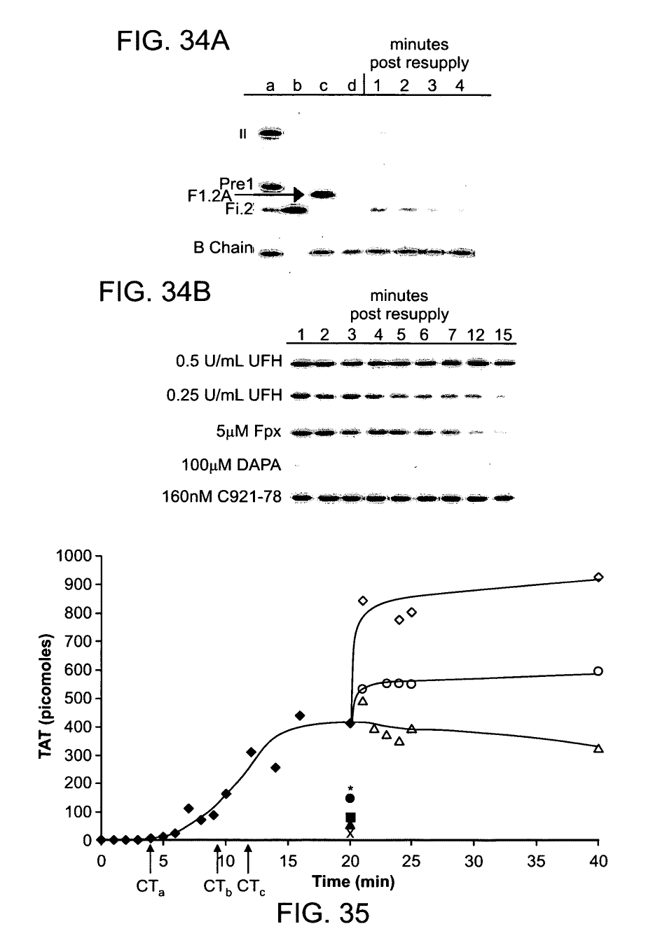

Prothrombin Consumption during Resupply—In FIGS. 32B, 32D and 33D, the estimates of UFH, Fpx and DAPA efficacy potentially represent a combination of 2 modes of anticoagulant action: their relative reactivity toward preformed catalytic complexes and their direct inhibition of thrombin formed during the reaction. A Western electrophoretic analysis detailing the time course of prothrombin consumption during the resupply response in the synthetic coagulation proteome is shown in FIGS. 34A, B. This analysis complements those presented in FIGS. 32-33, by directly displaying the effect of the anticoagulant on the performance of the prothrombin activating potential that drives the resupply response. FIG. 34A presents a resupply time course (truncated for presentation) in the absence of anticoagulant. Lanes (a to c) display the relative mobility of fII, reaction intermediates (F1.2A, pre-1) and end products (F1.2, B chain). Lane (d) thereof represents the 20 min time point, which was sampled immediately prior to resupply. It shows the expected absence of prothrombin. Since the primary antibody used does not recognize thrombin when complexed with AT (TAT), the major product species after 20 min is not detected. In the absence of anticoagulant, prothrombin consumption is greater than 95% complete at 1 min post resupply, with the increase in the B chain reflecting the presence of active thrombin immediately after resupply.

FIG. 34B presents a composite of 5 immunoblots, showing for each one only the lanes visualizing the changes in prothrombin concentration for 15 min following resupply. Prothrombin consumption can be seen to be most effectively blocked in the presence of 160 nM C921-78 and 0.5 U/mL UFH, consistent with the low levels of thrombin measured (FIGS. 32B, ▴ and 33B, ◯). Densitometric analyses (not shown) indicated that blockade by C921-78 was somewhat more effective, allowing ˜10% prothrombin consumption at 15 min compared to ˜20% in the presence of 0.5 U/mL UFH. Inhibition of prothrombin activation by UFH at 0.25 U/mL (12.5% occupancy of the available AT) appeared slightly less effective than that observed with 5 μM Fpx. (100% AT occupancy), with the time to 50% prothrombin consumption being about 1 min faster. This is in contrast to the activity based assessment (FIG. 32B, □ versus FIG. 32D, Fpx:▪) where thrombin formation appears more rapid in the presence of Fpx. As predicted computationally, DAPA anticoagulation proved completely ineffective in blocking prothrombin consumption in the resupply format.

Anticoagulant Efficacy in Whole Blood—To test whether results from the computational and proteome based analyses would extrapolate to a more complex empirical model, the efficacy of anticoagulants in suppressing thrombin generation was addressed in contact pathway inhibited blood in both Tf-initiated and resupply regimens. FIG. 35 presents a representative experiment assessing the performance of C921-78 and Fpx. The initial time course of TAT complex formation in the absence of any anticoagulant after stimulation with 5 pM Tf reagent is presented (♦). Subsets of blood aliquots were also treated with various concentrations of C921-78 or Fpx, initiated with 5 pM Tf, quenched at 20 min and 20 min TAT levels for each of these anticoagulant treated aliquots were established (FIG. 35, closed symbols at 20 min). These data also are summarized in the tabulation provided as FIG. 29.

Incorporation of increasing concentrations of C921-78 into blood prior to Tf initiation led to decreases in TAT levels at 20 min (FIG. 29). Suppression by C921-78 appears less robust in blood than in the synthetic coagulation proteome (e.g. FIG. 31B, ), probably because of nonspecific binding to plasma proteins. Incorporation of increasing concentrations of Fpx into blood prior to Tf addition also led to decreases in the TAT levels at 20 min (FIG. 29). Fpx efficacy in blood is in line with results in the synthetic coagulation proteome (e.g., FIG. 30D, Fpx: , ▴).

Ongoing Tf-initiated reactions without anticoagulant were resupplied at 20 min with blood containing no anticoagulant (⋄), C921-78 (160 nM final, Δ), or Fpx (1 μM final, ◯), and the time courses of TAT formation are shown. As predicted from both computational and synthetic coagulation proteome assessments, 1 μM Fpx (◯) proved relatively ineffective in suppressing thrombin generation in the resupply format when compared to 160 nM C921-78 (Δ). Thrombin generation in the presence of 1 μM Fpx reached approximately 40% of that in the absence of anticoagulant (FIG. 29). Immunoblots of these resupply time courses directed at prothrombin species (not shown) confirmed substantial prothrombin activation both in the absence of anticoagulants and the presence of 1 μM Fpx, while showing that prothrombin consumption was minimal in the presence of 160 nM C921-78. Thus, computational and proteome-based assessments in both prophylactic and therapeutic settings proved to be in reasonable accord with anticoagulant performance in contact pathway inhibited blood.

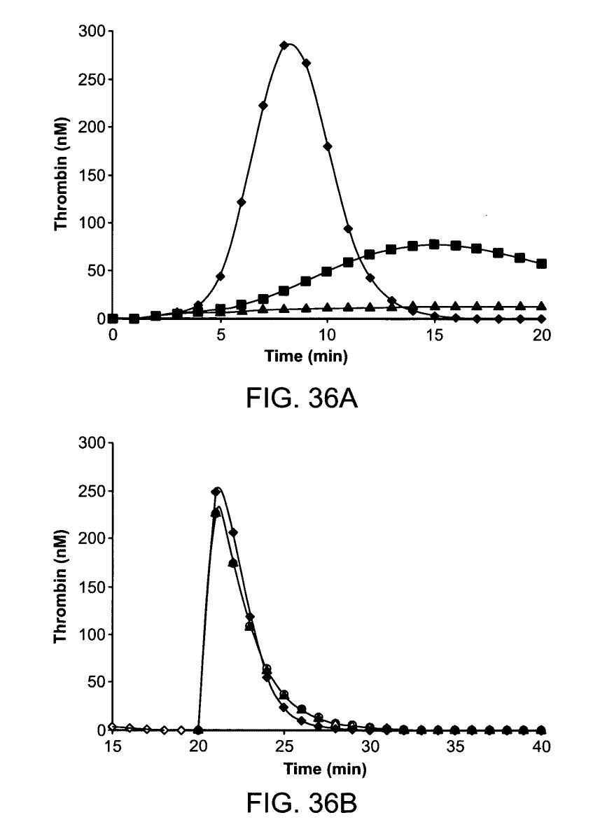

Prophylactic and Therapeutic Efficacy of a Hypothetical Direct FIXa Inhibitor—In limited clinical applications, fIX/IXa inhibitors have showed promise, achieving effective anti-coagulation and a reduced risk of bleeding compared to UFH(14). There is shown in FIGS. 36A,B computational assessments of the efficacy of a hypothetical direct fIXa inhibitor in suppressing both Tf-initiated thrombin formation (FIG. 36A) and thrombin formation following resupply (FIG. 36B). The interactions of this inhibitor with fIXa and fIXa in the intrinsic fXase complex were defined by the corresponding kinetic parameters describing C921-78 interactions with fXa and fXa in the prothrombinase complex (see FIG. 28, rate numbers 62-65 and the respective equations in FIG. 27). In the Tf-initiated, or prophylactic application, the hypothetical direct fIXa inhibitor was predicted to be similar to C921-78 in its capacity to suppress thrombin formation. At 10 nM (FIGS. 36A,B; ▪), thrombin levels reached only 13 nM at 20 min, compared to complete suppression observed with C921-78 (FIG. 31A, ). In contrast, in the resupply format, the direct fIXa inhibitor proved ineffective, achieving less than 10% suppression of thrombin formation at 40 nM (FIG. 36B, ◯) and no further effect at 160 nM (FIG. 36B, ▴). This differs markedly from the progressive and effective suppression of thrombin formation after resupply predicted for the same concentration range of C921-78 (FIG. 33A). Thus, direct fIXa inhibitors are predicted to be effective for prophylactic but not therapeutic use.

### Example 3

An assessment was made of protein (C) using the methodology of the present invention for a predetermined population. The model embodying such methodology was run to evaluate risk prediction with and without the PC pathway influence and/or to evaluate the thrombin generation profiles in carriers of the common genetic defects in prothrombin G20210A, factor V Leiden and factor xIII Val34Leu. Dr. Simon Body, at the Brigham and Women's Hospital in Boston, has a cohort of 1700 patients who underwent coronary artery bypass surgery and for whom have a recorded postoperative blood loss or thrombosis. We evaluated TFPI for them, and in collaboration will evaluate computational thrombin generation in a thrombosis and bleeding cohort based upon their FVIII, FII, TFPI and AT levels.

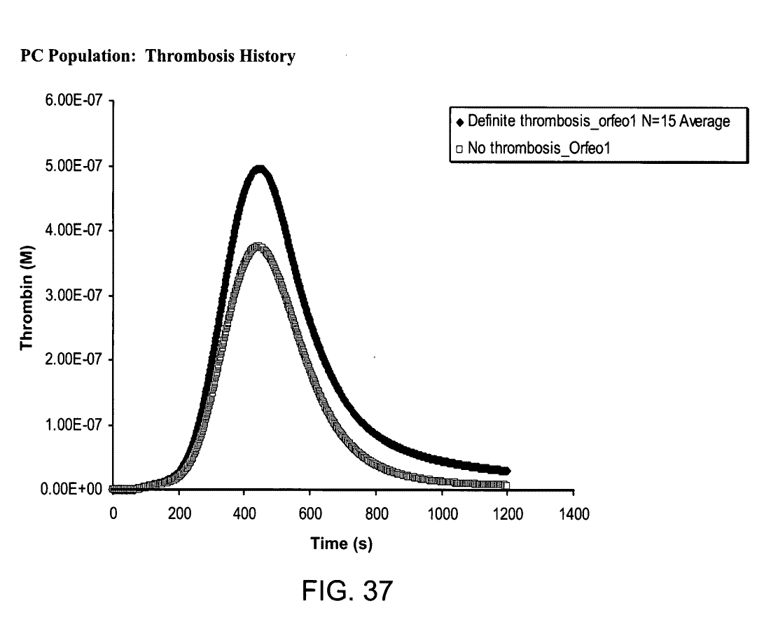

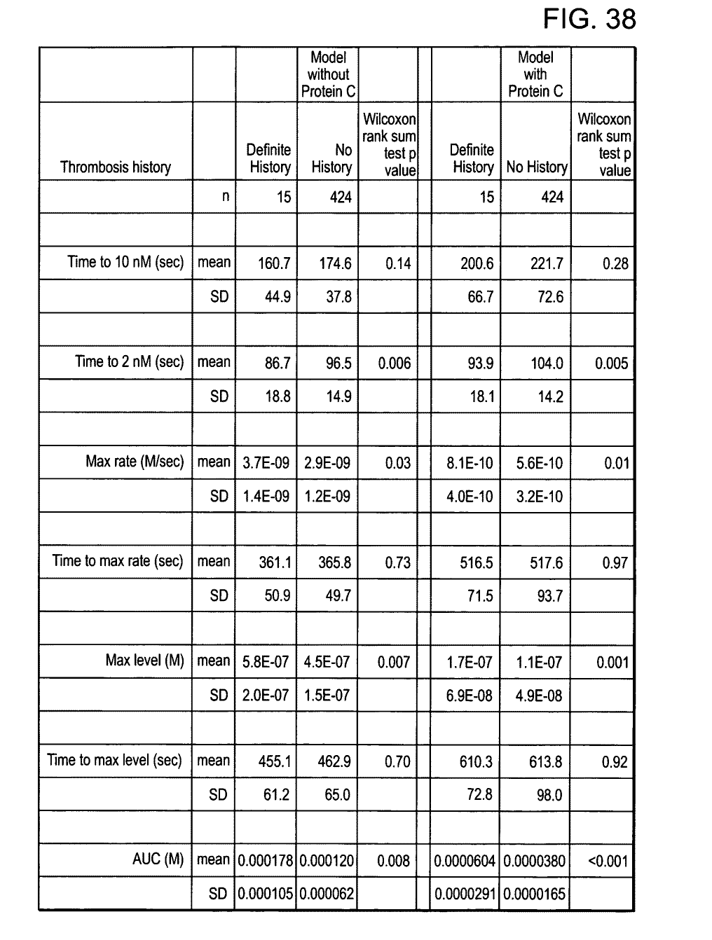

The results of such assessment are presented in FIGS. 37-38.

### Example 4

Hemophilia A Hemophilia A, an X-linked disease with an estimated prevalence of I in 10,000 males [Hoyer L W. Hemophilia A. N Engl J Med 1994; 330: 38-47], is characterized by the decrease or absence of functional FVIII. It is one of the most extensively studied hemorrhagic disorders, ranging from clinical observations and treatment regimens, from biochemical and molecular analyses, to investigative gene therapy. Within the hemophilia A population, phenotypic heterogeneity with respect to clinical severity is seen. Although, the potential to bleed is diagnosed by traditional coagulation assays the hemorrhagic pathology and its' clinical management are not always accurately predicted by these same assays. In fact, it is estimated that 10% of severe hemophilia A patients (<1% FVIII) have only mild bleeding diathesis despite the biochemically undetectable levels of FVIII [Aledort L. Why thrombin generation? From bench to bedside. Pathophysiol Haemost Thromb 2003; 33: 2-3; Ahlberg A. Haemophilia in Sweden. VII. Incidence, treatment, and prophylaxis of arthropathy and other musculoskeletal manifestations of hemophilia A and B. Acta Orthop Scand 1965; 77: 3-132]. The question, “Why does the level of FVIII not always correlate with clinical manifestation?” remains unanswered. New investigative methods need to be developed to understand hemorrhagic risk in the individual hemophilia patient. We believe that all the factor levels together act synergistically to generate individualized thrombin profiles that can be translated into a pattern to predict hemorrhagic risk.

### Thrombin Generation and Bleeding in Hemophilia A

Using our CTI-inhibited whole blood model, we studied 11 mild (6%-40% FVIII:C), 4 moderate (≦2%-5% FVIII:C) and 12 severe (<1% FVIII:C) FVIII deficient individuals with a well-characterized 5-year bleeding history that included hemarthrosis, soft tissue hematoma and annual FVIII concentrate usage. This clinical information was used to generate a bleeding score separated into three groups and compared to thrombin generation outputs. Our results showed that the maximum level of thrombin formed was significantly different (P<0.001) between the groups: 504 nM (SD114), 315 nM (SD117) and 194 nM (SD91); with higher thrombin concentrations in the groups with lower bleeding scores. This empirical study in CTI-inhibited whole blood showed that thrombin generation appears to be associated with the bleeding phenotype of hemophilia A.

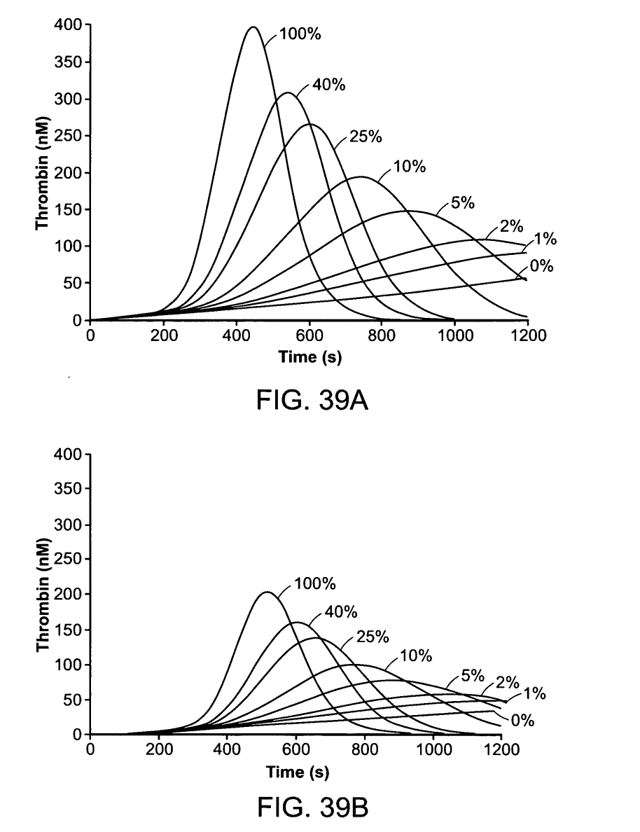

Since levels of thrombin generation in the empirical model have been shown to be related to overall factor levels, it would suggest that the bleeding phenotype of hemophilia A is also influenced by each individual's ensemble of clotting factors. Therefore, we carried out a hypothetical analysis of the influence of factor levels by varying FVIII on thrombin generation with a constant Tf-stimulus. We modeled a FVIII titration of 0%, 1%, 2%, 5%, 10%, 25%, 40% and 100% FVIII, with all other factor values reflecting either clinically accepted high or low normal values for each procoagulant and anticoagulant. There is shown in FIG. 39A, factor levels represent the high extreme of the normal range and FIG. 39B represents the low extreme of the normal range for each factor. For example when a FVIII deficient individual has 5% FVIII, if their other factor levels are at the high extreme of the normal range, they will be able to generate thrombin faster and reach maximum levels quicker than a 5% FVIII individual with their other factor levels at the low extreme of the normal range. This result suggests a rationale for why hemophiliacs with the same FVIII level, whether of therapeutic or natural origin, can differ hemostatically.

### CONCLUSION

There is a growing consensus that global assessments of blood coagulation performance are essential to understanding individual variability. The limitation of empirical approaches in general is that while one can get a significant insight into an individual's coagulant response via blood or plasma sample, the mechanism behind that individual response is not elucidated by these methods. Using a combination of empirical methods and a computational approach based upon a mechanistic description of Tf-initiated coagulation allows one to explore potential explanations for the observed variability among individuals.

The central idea is that in any individual, procoagliant and anticoagulant factor levels act together to generate a unique coagulation phenotype represented by their thrombin generation profile. Three complementary hypotheses have emerged from our work: (i) compensation by the ensemble of other coagulation proteins in individuals with specific factor deficiencies can ‘normalize’ an individual's thrombin generation process and that this effect represents a rationale for their unexpected phenotype; (ii) individuals with clinically unremarkable factor levels may present thrombin generation profiles typical of individuals with hemostatic complications; and (iii) in some hemostatic disorders a specific pattern of expression of a small ensemble of coagulation factors may be sufficient to explain the overall phenotype.

### Example 5

In vitro fertilization (IVF) is a well-documented risk factor for thromboembolic complications [Chan W S, Dixon M E. The “ART” of thromboembolism: A review of assisted reproductive technology and thromboembolic complications. Thromb Res 2008; 121:713-26]. Suspected complications have included jugular venous thrombosis, and fatal cerebral infarction. Possible plasma factor compositional reasons for the link between IVF and thrombotic events include activated protein C resistance, decreased protein S and antithrombin (AT), and increased fibrinogen and factor (F) VIII [Biron C, Galtier-Dereure F, Rabesandratana H. Bernard I, Aguilar-Martinez P, Schved J F, et al. Hemostasis parameters during ovarian stimulation for in vitro fertilization: results of a prospective study. Fertil Steril 1997; 67: 104-9; Curvers J, Nap A W, Thomassen M C, Nienhuis S J, Hamulyak K, Evers J L, et al. Effect of in vitro fertilization treatment and subsequent pregnancy on the protein C pathway. Br J Haematol 2001; 115:400-7; Aune B, Hoie K E, Oian P, Holst N, ↑sterud B. Does ovarian stimulation for in-vitro fertilization induce a hypercoagulable state? Hum Reprod 1991; 6:925-7; Michelon J, Petracco A, Bruxel D M, Moretto M, Badalotti M. [Analysis of coagulation parameters in patients undergoing controlled ovarian hyperstimulation for in vitro fertilization]. Rev Assoc Med Bras 2002; 48:345-7; Bremme K, Wramsby H, Andersson O, Wallin M, Blomback M. Do lowered factor VII levels at extremely high endogenous oestradiol levels protect against thrombin formation? Blood Coagul Fibrinolysis 1994; 5:205-10]. These factors could potentiate increased thrombin generation.

Generation of thrombin, a key enzyme in blood coagulation, has been suggested as a potential marker of risk evaluation. Previous studies on citrated plasma using calibrated automated thrombography showed that women using oral contraceptives have shorter thrombin generation lag times and higher peak height. In a computational and empirical study, women on oral contraceptives also had an increased rate and maximum level of thrombin generation in response to a 5 pM tissue factor (TF) stimulus than women not using oral contraceptives [Brummel-Ziedins K E, Vossen C Y, Butenas S, Mann K G, Rosendaal F R. Thrombin generation profiles in deep venous thrombosis. J Thromb Haemost 2005; 3: 2497-505]. In addition, Harnett et al. noticed a significant decrease in clot time in women undergoing IVF using thrombelastography [Harnett M J, Bhavani-Shankar K, Datta S, Tsen L C. In vitro fertilization-induced alterations in coagulation and fibrinolysis as measured by thromboelastography. Anesth Analg 2002; 95:1063-6 table].

In this study, we investigated the effect of high levels of endogenous estrogen on plasma compositional influence on thrombin generation in seven women receiving follicle stimulating hormone in preparation for IVF. Blood was drawn on two separate occasions. Baseline studies were obtained on menstrual cycle day 2 or 3, one month prior to the start of an IVF cycle. A second blood draw took place on the final day of stimulation when estradiol levels would be at their peak. Estradiol rose during treatment (29 pg/mL (SD 12) to 2773 pg/mL (SD 2076) and successful pregnancy occurred in five of seven cases. Factor levels were measured on each of the individuals before and after stimulation for the coagulation proteins FII, FV, FVII, FVIII, FIX, FX, AT and total tissue factor pathway inhibitor (TFPI). FII, FV, FVII, FVIII, FIX and FX were measured by using a Stago STA-R, in which the subjects plasma is added to plasma deficient in the factor to be measured and the degree of correction of clotting time is determined. The degree of the correction is compared with that obtained by adding a normal plasma to the same system. AT is measured using a STA-Stachrom AT Colorimetric assay and total TFPI was measured by ELISA (Diagnostica Stago, France). Factor levels expressed as a percent of normal were translated into molar (M) concentrations using literature values for the mean plasma concentrations [16]. The Wilcoxon signed rank test was utilized for all comparisons, where a p-value ≦0.05 was considered significant. All data are presented as the mean (SD).

Studying patients in IVF cycles permits the observation of thrombin generation over a wide range of estradiol levels. As estradiol rose, protein concentrations were within the normal range. Significant differences after stimulation in coagulation factor concentrations included decreases in FV (25 nM (SD 2) to 21 nM (SD 3); p=0.02) and AT (3.6 μM (SD 0.4) to 3.2 μM (SD 0.3); p=0.05). Other non-significant changes were: a decrease in FII (1.5 μM (SD 0.18) to 1.4 μM (SD 0.18); p=0.12) and TFPI (1.8 nM (SD 0.5) to 1.5 nM (SD 0.3); p=0.08) and an increase in FVIII (1.0 nM (SD 0.2) to 1.2 nM (SD 0.4); p=0.30). Similar levels were observed in FVII (from 9.2 nM (SD 0.9) to 9.2 DM (SD 1.5); p=1.00), FIX (89 nM (SD 14) to 92 nM (SD 14); p=0.61) and FX (181 nM (SD 22) to 185 nM (SD 22); p=0.59).

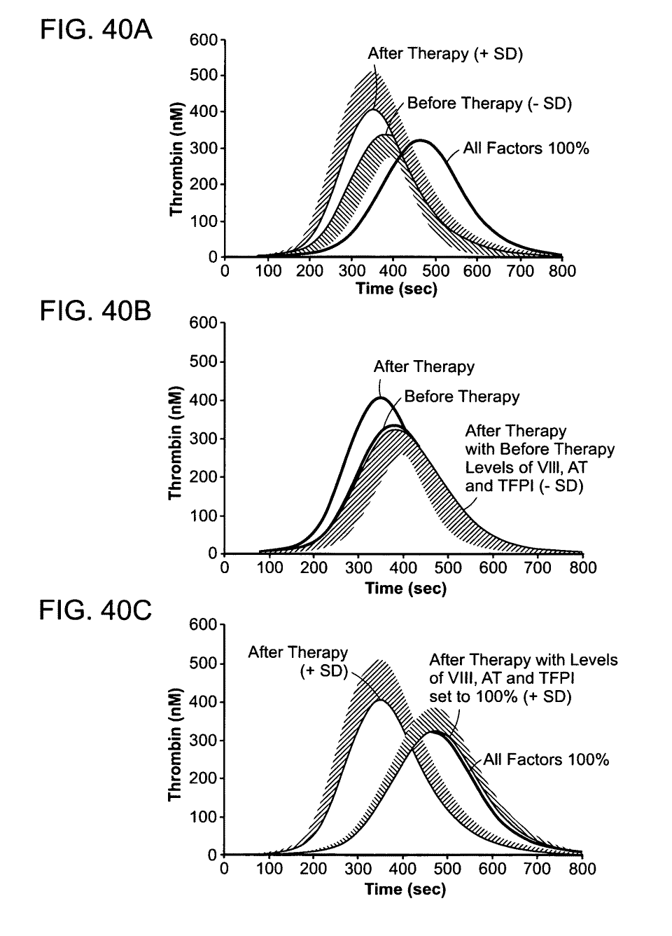

We modeled hypothetical TF-induced thrombin generation utilizing a deterministic simulation system for blood coagulation dynamics. Thrombin generation was evaluated by parameters that reflect the global qualities of the thrombin profile which includes: time to 10 nM thrombin (clot time, CT), maximum level (MaxL) and rate (MaxR) of thrombin generation and the times at which they are reached, TMaxL and TMaxR, respectively. Before treatment, the mean simulated thrombin generation profile for the group was shifted in the hypercoagulable direction relative to a mean profile where all factors are at 100% of the mean physiologic concentration (FIG. 40A). In six of the seven women, the increased hormone levels coincided with a shift towards a faster and more prominent thrombin generation profile after IVF.

At high levels of endogenous estradiol, the CT decreased (151 s (SD 26) to 129 s (SD 12); p=0.03) and the MaxL increased (382 nM (SD 5) to 444 nM (SD 89); p=0.05) with a shortened TMaxL (394 s (SD 46) to 355 s (SD 34); p=0.02). Likewise, the MaxR increased (2.90 nM/s (SD 0.66) to 3.76 ns (SD 1.03); p=0.03) and the TMaxR decreased (312 s (SD 36) to 281 s (SD 26); p=0.02). In addition, the concentration of estradiol after treatment was positively correlated (r²=0.86) with the MaxL. Although this is a limited study, the two individuals who were not successful in becoming pregnant had the highest MaxL and the fastest CT. These findings are in agreement with Rogolino et al. who observed significantly higher thrombin-antithrombin levels in ovarian hyperstimulation syndrome patients who did not become pregnant [Rogolino A, Coccia M E, Fedi S, Gori A M, Cellai A P, Scarselli G F, et al. Hypercoagulability, high tissue factor and low tissue factor pathway inhibitor levels in severe ovarian hyperstimulation syndrome: possible association with clinical outcome. Blood Coagul Fibrinolysis 2003; 14:277-82].

The influence of each coagulation protein (or set of proteins) on thrombin generation was analyzed by systematically running independent thrombin generation simulations. Each simulation had one (or more) of the proteins set to the level it was prior to gonadotropin stimulation, while leaving all others at post-stimulation levels. In four of the six women who had their profile shift in a prothrombotic direction, adjusting the after stimulation levels of FVIII, AT and TFPI to the levels they were at before stimulation, resulted in near identical thrombin generation curves to those generated when all proteins were at before stimulation (FIG. 40B). Despite the fact that pre- to post-gonadotropin stimulation differences in FV, FVII, FIX and FX existed, adjustments of these factors did not affect the thrombin generation curve. Additionally, adjusting the after gonadotropin stimulation levels of FVIII, TFPI and AT to their mean physiologic concentrations (while leaving other factors at their after stimulation level) produces a thrombin generation curve similar to a theoretical control curve where values are set at mean physiologic concentrations (FIG. 40C).

These studies generate hypothetical thrombin generation profiles which do not include the contribution of the anticoagulant protein C pathway, the contact pathway and the vasculature. The numerical model that was used for these studies includes key plasma pro- and anti-coagulants of the TF pathway to thrombin generation that are evaluated in clinical laboratories. This model correlates well to empirical models describing the TF pathway [Hockin M F, Jones K C, Everse S J, Mann K G. A model for the stoichiometric regulation of blood coagulation. J Biol Chem 2002; 277: 18322-33; Brummel-Ziedins K, Rivard G E, Pouliot R I, Butenas S, Gissel M, Parhami-Seren B, et al. Factor VIIa replacement therapy in factor VII deficiency. J Thromb Haemost 2004; 2: 1735-44; Orfeo T, Brummel-Ziedins K E, Gissel M, Butenas S, Mann K G. The nature of the stable blood clot procoagulant activities. J Biol Chem 2008; 283: 9776-86; Butenas S, Orfeo T, Brummel-Ziedins K E, Mann K G. Influence of bivalirudin on tissue factor-triggered coagulation. Blood Coagul Fibrinolysis 2007; 18: 407-14].

In conclusion, women receiving IVF treatment in this pilot study who have normal but slightly prothrombotic thrombin generation profiles responded to high levels of hormones by becoming more prothrombotic. A systematic analysis of the plasma coagulation factors has isolated FVIII, AT and TFPI as the components most responsible for the additional procoagulant shift. This pattern of small concerted changes in the triad of FVIII, AT and TFPI levels may contribute to the prothrombotic state in some women undergoing IVF.

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method for determining hemostatic risk in a subject, the method comprising the steps of:
determining the concentrations of a plurality of blood factors in a biologic sample from the subject;
simulating in silico the concentration of thrombin from the determined concentrations; and
comparing the simulated concentration of thrombin to a reference by a clinician, and
determining from the simulated concentration if the subject is predisposed to hemostatic risk.

2. The method of claim 1, wherein said determining includes determining the concentrations of three blood factors in a biologic sample from the subject;

3. The method of claims 1 or 2, wherein the blood factors are selected from the group consisting of AT, FII, FVIII, Protein C, Protein S, Factor V^{Leiden}, and tissue factor pathway inhibitor (TFPI).

4. The method of claim 2, wherein the three blood factors are AT, FII, and FVII.

5. The method of claim 2, wherein the three blood factors are Factor V^{Leiden}, Protein C and Protein S.

6. The method of claim 2, wherein the three blood factors are TFPI, AT and FVIII.

7. The method of claim 1, wherein the hemostatic risk is one of ACS or hemophilia.

8. The method of claim 1, further comprising the step(s) of:
after determining that the subject is predisposed to hemostatic risk, determining a prophylactic treatment to minimize the hemostatic risk to the subject.

9. The method of claim 8, wherein said method further includes the steps of:
after determining the prophylactic treatment, inputting parameters representative of the capacity of the prophylactic treatment to modulate at least one blood factor;
repeating said simulating in silico the concentration of thrombin from the determined concentrations and the modulating effect of the determined prophylactic treatment; and
assessing by the clinician the efficacy of the determined prophylactic treatment to minimize the hemostatic risk;
if said assessment provides a satisfactory indication of efficacy, proscribing the prophylactic treatment; and
if said assessment provides an unsatisfactory indication of efficacy; selecting another prophylactic treatment and repeating said steps of inputting. said repeating said simulating and said assessing for the another prophylactic treatment.

10. The method of claims 8 or 9, wherein the prophylactic treatment is at least one of drugs, medicaments, dietary and physical therapy.

11. The method of claim 10, wherein the drug modulates at least one of the procoagulant factor or the anticoagulant factor of the subject.

12. The method of claim 1, wherein said simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation.

13. The method of claim 12, wherein the computer executable functions include at least one of the following variables: 1) TFPI mediated inactivation of TF•VIIa and its product complexes; 2) AT-III mediated inactivation of IIa, mIIa, factor VIIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

14. A method of claim 1, wherein the hemostatic risk is ACS, and wherein said comparing includes comparing the simulated concentration of thrombin to a reference by the clinician, and determining from the simulated concentration if the subject is predisposed to ACS, wherein a simulated thrombin concentration within one standard deviation of the reference indicates that the subject is predisposed to ACS.

15. The method of claim 14, wherein said determining includes determining the concentrations of three blood factors in a biologic sample from the subject;

16. The method of claims 14 or 15, wherein the blood factors are selected from the group consisting of AT, FII, FVIII, Protein C, Protein S, Factor V^{Leiden}, and tissue factor pathway inhibitor (TFPI).

17. The method of claim 15, wherein the three blood factors are AT, FII, and FVIII.

18. The method of claim 14, wherein said method further includes the steps of:
after determining a prophylactic treatment, inputting parameters representative of the capacity of the prophylactic treatment to modulate at least one blood factor;
repeating said simulating in silico the concentration of thrombin from the determined concentrations and the modulating effect of the determining a prophylactic treatment; and
assessing the efficacy of the determined prophylactic treatment to minimize the hemostatic risk for ACS by the clinician;
if said assessment provides a satisfactory of efficacy for the determined prophylactic treatment; proscribing such prophylactic treatment; and
if said assessment provides an unsatisfactory indication of efficacy; altering the determined prophylactic treatment and repeating said steps of inputting. said repeating said simulating and said assessing.

19. The method of claim 18, wherein the prophylactic treatment is at least one of drugs, medicaments, dietary and physical therapy.

20. The method of claim 19, wherein the drug modulates at least one of the procoagulant factor or the anticoagulant factor of the subject.

21. The method of claim 14, wherein said simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation.

22. The method of claim 21, wherein the computer executable functions include at least one of the following variables: 1) TFPI mediated inactivation of TF-VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

23. A method for selecting a treatment in a subject, the method comprising the steps of:
simulating in silico the concentration of thrombin from the concentrations of a plurality of blood factors in a biologic sample from the subject;
comparing the simulated concentration of thrombin to a reference; and
selecting a treatment based on said comparing.

24. The method of claim 23, wherein said determining includes determining the concentrations of three blood factors in a biologic sample from the subject;

25. The method of claims 23 or 24, wherein the blood factors are selected from the group consisting of AT, FII, FVIII, Protein C, Protein S, Factor V^{Leiden}, and tissue factor pathway inhibitor (TFPI).

26. The method of claim 24, wherein the three blood factors are one of (a) AT, FII, and FVIII; (b) Factor V^{Leiden}, Protein C and Protein S; or (c) are TFPI, AT and FVIII.

27. The method of claim 23, wherein said method further include:
after determining the treatment, inputting parameters representative of the capacity of the determined treatment to modulate at least one blood factor;
repeating said simulating in silico the concentration of thrombin from the plurality of blood factor concentrations and the modulating effect of the determined treatment; and
assessing by the clinician of the efficacy of the determined treatment to minimize hemostatic risk;
if said assessment provides a satisfactory indication of efficacy, proscribing such treatment; and
if said assessment provides an unsatisfactory indication of efficacy; selecting another treatment and repeating said steps of inputting. said repeating said simulating and said assessing for the another treatment.

28. The method of claim 27, wherein the determined treatment is at least one of drugs, medicaments, dietary and physical therapy.

29. The method of claim 28, wherein the drug modulates at least one of the procoagulant factor or the anticoagulant factor of the subject.

30. The method of claim 23, wherein said simulating in silico includes performing a series of computer executable functions that manipulate input data featuring at least one of blood coagulation formation, expression and propagation variables, the functions generating, as output, a thrombin concentration, wherein the amount of thrombin is taken to be indicative of the blood coagulation and wherein the computer executable functions include at least one of the following variables: 1) TFPI mediated inactivation of TF•VIIa and its product complexes; 2) AT-III mediated inactivation of IIa, mIIa, factor VIIa, factor IXa, and factor Xa; 3) initial activation of factor V and factor VIII by thrombin generated by factor Xa-membrane; 4) factor V inactivation by activated Protein C pathway; 5) factor VIIIa dissociation/activity loss; 6) binding competition, and kinetic activation steps which exist between tissue factor (TF) and factors VII and VIIa, and 7) activation of factor VII by IIa, factor Xa, and factor IXa.

31. A method for diagnosing ACS in a subject, the method comprising the steps of:
determining the concentrations of AT, FII, and FVIII in a biologic sample from the subject;
simulating in silico the concentration of thrombin from the concentrations of AT, FII, and FVIII; and
comparing the simulated concentration of thrombin to a reference, wherein a simulated thrombin concentration within one standard deviation of the reference indicates that the subject has ACS.
